(19) United States
(12) Patent Application Publication
(10) Pub. No.: US 2024/0042025 A1
(43) Pub. Date: Feb. 8, 2024

(54) BIALLELIC KNOCKOUT OF B2M

(71) Applicants: Lior IZHAR, Tel Aviv (IL); Rafi EMMANUEL, Ramla (IL); EmendoBio Inc., Wilmington, DE (US)

(72) Inventors: Lior Izhar, Tel Aviv (IL); Rafi Emmanuel, Ramla (IL)

(73) Assignee: EmendoBio Inc., Wilmington, DE (US)

(21) Appl. No.: 18/257,181

(22) PCT Filed: Dec. 14, 2021
(86) PCT No.: PCT/US2021/063302

§ 371 Date: Jun. 13, 2023

Related U.S. Application Data
(60) Provisional application No. 63/125,214, filed on Dec. 14, 2020.

(51) Int. Cl. A61K 39/00; C12N 15/11; C12N 9/22; C12N 15/90

(52) U.S. Cl. CPC A61K 39/4621; C12N 15/11; C12N 9/22; C12N 15/907; A61K 39/4613; A61K 39/4612; A61K 39/4611; A61K 39/4614; C12N 2310/20; C12N 2800/80

(57) ABSTRACT

RNA molecules comprising a guide sequence portion having 17-50 contiguous nucleotides containing nucleotides in the sequence set forth in any one of SEQ ID NOs: 1-1538 and compositions, methods, and uses thereof.

This application claims the benefit of U.S. Provisional Application No. 63/125,214, filed Dec. 14, 2020, the content of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “211214 91654-A-PCT Sequence Listing AWG.txt”, which is 453 kilobytes in size, and which was created on Dec. 14, 2021 in the IBM-PC machine format, having an operating system compatibility with MS-Windows, which is contained in the text file filed Dec. 14, 2021 as part of this application.

### BACKGROUND OF INVENTION

Chimeric antigen receptors (CAR) provide a promising approach for immunotherapy. However, to make such therapies (e.g. CAR-T cell therapies) more accessible, it is highly desirable to develop an allogeneic adoptive transfer strategy in which universal CAR cells derived from cells from healthy donors can be applied to treat multiple patients. In order to implement this strategy, the αβ T-cell receptor (TCR) on allogenic CAR-T cells needs to be eliminated by knocking out T Cell Receptor Alpha Constant (TRAC) in order to avoid graft-versus-host-disease (GVHD). Additionally, to minimize the immunogenicity of CAR-T cells human leukocyte antigens class I (HLA-Is) need to be removed by knocking out beta-2 microglobulin (B2M).

### SUMMARY OF THE INVENTION

Disclosed are approaches for knocking out the B2M gene in cells to be utilized for immunotherapy approaches, such as CAR-T therapy. The B2M knockout lowers the immunogenicity of the modified cell to avoid rejection by the host's immune system during allogeneic adoptive transfer. Accordingly, biallelic knockout of the B2M gene in cells as described herein may be utilized to minimize the immunogenicity of the cells. The disclosed approaches for knocking out the B2M gene in cells, for example, allogeneic immune cells, are utilized to increase the persistence and/or the engraftment of the cells in the presence of a host's immune cells.

The present disclosure also provides a method for inactivating alleles of the Beta-2 microglobulin (B2M) gene in a cell, the method comprising
introducing to the cell a composition comprising:
   a CRISPR nuclease or a sequence encoding the CRISPR nuclease; and
   an RNA molecule comprising a guide sequence portion having 17-50 nucleotides or a nucleotide sequence encoding the same,
wherein a complex of the CRISPR nuclease and the RNA molecule affects a double strand break in the allele of the B2M gene.

According to embodiments of the present invention, there is provided an RNA molecule comprising a guide sequence portion having 17-50 contiguous nucleotides containing nucleotides in the sequence set forth in any one of SEQ ID Nos: 1-1538.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-1538 and a CRISPR nuclease.

According to some embodiments of the present invention, there is provided a method for inactivating a B2M allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-1538 and a CRISPR nuclease. In some embodiments, the cell is a lymphocyte. In some embodiments, the cell is a T cell. In some embodiments, the cell is a T regulatory cell. In some embodiments, the cell is a B cell. In some embodiments, the cell is a natural killer (NK) cell. In some embodiments, the cell is a macrophage. In some embodiments, the cell is a stem cell. In some embodiments, the cell is an iPSC. In some embodiments, the cell is a fibroblast, blood cell, hepatocyte, keratinocyte, or any other cell type capable of being reprogrammed to an induced pluripotent stem cell (iPSC). In some embodiments, the delivering to the cell is performed in vivo, ex vivo, or in vitro. In some embodiments, the method is performed ex vivo and the cell is provided/explanted from an individual patient. In some embodiments, the method further comprises the step of introducing the resulting cell, with the modified/knocked out B2M allele, into an individual patient. In some embodiments, the cell is originated from the individual patient to be treated. In some embodiments, the cell originated from a donor. In some embodiments, the cell is allogeneic to the individual patient to which it is introduced.

According to some embodiments of the present invention, there is provided a method for treating, reducing, and/or preventing host-versus-graft rejection (HvG), the method comprising delivering to a cell of a subject having or at risk of experiencing host-versus-graft rejection (HvG) a composition comprising an RNA molecule comprising a guide sequence portion having 17-50 contiguous nucleotides containing nucleotides in the sequence set forth in any one of SEQ ID NOs: 1-1538 and a CRISPR nuclease. In some embodiments, the method is for increasing persistence and/or engraftment of a cell in a host subject, the method comprising delivering to the cell a composition comprising an RNA molecule comprising a guide sequence portion having 17-50 contiguous nucleotides containing nucleotides in the sequence set forth in any one of SEQ ID NOs: 1-1538 and a CRISPR nuclease; and introducing the cell to the host subject. In some embodiments, the cell is further differentiated prior to introducing the cell to the host subject. In some embodiments, the cell is further engineered to express a chimeric antigen receptor. In some embodiments, the cell is a stem cell, an iPSC, or a progenitor cell and is differentiated to a T cell prior to introducing the cell to the host subject. In some embodiments, the cell is a T cell. In some embodiments, the cell is further engineered to knockout the TRAC gene to avoid graft-versus-host disease (GVHD) following introduction into a host subject.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-1538 and a CRISPR nuclease for inactivating a B2M allele in a cell, comprising delivering to the cell the composition comprising an RNA molecule comprising a guide sequence portion having 17-50 contiguous nucleotides containing nucleotides in the sequence set forth in any one of SEQ ID NOs: 1-1538 and a CRISPR nuclease.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-1538 and a CRISPR nuclease for use in inactivating a B2M allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-1538 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-1538 and a CRISPR nuclease for treating, ameliorating, or preventing host-versus-graft rejection (HvG) or increasing persistence of the cell upon engraftment, comprising delivering to a cell of a subject having or at risk of experiencing host-versus-graft rejection the composition of comprising an RNA molecule comprising a guide sequence portion having 17-50 contiguous nucleotides containing nucleotides in the sequence set forth in any one of SEQ ID NOs: 1-1538 and a CRISPR nuclease.

According to some embodiments of the present invention, there is provided a medicament comprising the composition comprising an RNA molecule comprising a guide sequence portion having 17-50 contiguous nucleotides containing nucleotides in the sequence set forth in any one of SEQ ID NOs: 1-1538 and a CRISPR nuclease for use in treating, ameliorating, or preventing host-versus-graft rejection (HvG), wherein the medicament is administered by delivering to a cell of a subject having or at risk of experiencing host-versus-graft rejection (HvG) the composition comprising an RNA molecule comprising a guide sequence portion having 17-50 contiguous nucleotides containing nucleotides in the sequence set forth in any one of SEQ ID NOs: 1-1538 and a CRISPR nuclease.

According to some embodiments of the present invention, there is provided a kit for inactivating a B2M allele in a cell, comprising an RNA molecule comprising a guide sequence portion having 17-50 contiguous nucleotides containing nucleotides in the sequence set forth in any one of SEQ ID NOs: 1-1538, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating or preventing host-versus-graft rejection (HvG) in a subject, comprising an RNA molecule comprising a guide sequence portion having 17-50 contiguous nucleotides containing nucleotides in the sequence set forth in any one of SEQ ID NOs: 1-1538, a CRISPR nuclease, and/or a tracrRNA molecule; and instructions for delivering the RNA molecule; CRISPR nuclease, and/or the tracrRNA to a cell of a subject having or at risk of host-versus-graft rejection (HvG).

### BRIEF DESCRIPTION OF THE DRAWINGS

FIGS. 1A-1B: Editing activity of OMNI CRISPR nucleases targeting B2M in HeLa cells. Specific guides were co-transfected with an OMNI CRISPR nuclease to determine their on-target activity. The PAM requirements of every nuclease were obtained by in vitro TXTL assay and the guide used with each nuclease is displayed in the x-axis labeling. Briefly, cells were harvested 72 h post DNA transfection, genomic DNA was extracted, and the region of the mutation was amplified and analyzed by next-generation sequencing (NGS). The graph represents the percentage (%) of editing. FIG. 1A: Batches 5.1 and 5.2. FIG. 1B: Batch 5.3.

FIG. 2: Guide activity of OMNI-103 CRISPR nuclease guides targeting B2M in HeLa cells. Specific guides were-co-transfected with OMNI-103 to determine their on-target activity. Cells were harvested 72 h post DNA transfection, genomic DNA was extracted, and the region of the mutation was amplified and analyzed by NGS. Transfection efficiency is measured by mCherry fluorescence. The graph represents the % of editing±standard deviation (STDV) (performed in triplicate).

FIG. 3: OMNI-103 CRISPR nuclease B2M editing activity as ribonucleoprotein (RNP) in U2OS a cell system. RNPs complexes of OMNI-103 and a specific guide molecule were electroporated into U2OS cells to determine their editing activity. Cells were harvested 72 h post RNP electroporation, genomic DNA was extracted, and the region of the mutation was amplified and analyzed by NGS. The graph represents the % of editing±STDV of two (2) independent electroporations.

FIG. 4: OMNI-103 CRISPR nuclease editing effect on protein levels of B2M in primary T cells. RNPs complexes of OMNI-103 and a specific guide molecule were electroporated into activated primary T cells. Cells were harvested eight (8) days post RNP electroporation, and cells were collected and analyzed by a FACS assay.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence.

As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-50, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-1538. Each possibility represents a separate embodiment. In some of these embodiments, the guide sequence portion comprises a sequence that is the same as a sequence set forth in any of SEQ ID NOs: 1-1538. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common to all alleles of a gene.

In embodiments of the present invention, an RNA molecule comprises a guide sequence portion having 17-50 contiguous nucleotides containing nucleotides in the sequence set forth in any one of SEQ ID NOs: 1-1538.

The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 17-50 nucleotides in length and contain 20-22 contiguous nucleotides in the sequence set forth in any one of SEQ ID NOs: 1-1538. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-1538. For example, a guide sequence portion having 17 nucleotides in the sequence of 17 contiguous nucleotides set forth in SEQ ID NO: 1539 may consist of any one of the following nucleotide sequences (nucleotides excluded from the contiguous sequence are marked in strike-through):

(SEQ ID NO: 1539)
AAAAAAAUGUACUUGGUUCC
17 nucleotide guide sequence 1:
(SEQ ID NO: 1540)
AAAAUGUACUUGGUUCC
17 nucleotide guide sequence 2:
(SEQ ID NO: 1541)
AAAAAUGUACUUGGUUC
17 nucleotide guide sequence 3:
(SEQ ID NO: 1542)
AAAAAAUGUACUUGGUU
17 nucleotide guide sequence 4:
(SEQ ID NO: 1543)
AAAAAAAUGUACUUGGU

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-1538 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided a method for inactivating alleles of the Beta-2 microglobulin (B2M) gene in a cell, the method comprising
introducing to the cell a composition comprising:
   at least one CRISPR nuclease, or a sequence encoding a CRISPR nuclease; and
   an RNA molecule comprising a guide sequence portion,
wherein a complex of the CRISPR nuclease and the RNA molecule affects a double strand break in alleles of the B2M gene,
wherein the guide sequence portion of the RNA molecule comprises 17-50 contiguous nucleotides containing nucleotides in the sequence set forth in any one of SEQ ID NOs: 1-1538.

In some embodiments, the guide sequence portion of the RNA molecule comprises 17-50 contiguous nucleotides containing nucleotides in the sequence set forth in any one of SEQ ID NOs: 1-1538 modified to comprise up to five mismatches relative to the target site.

In some embodiments, the method is a method of preparing modified immune cells (e.g., T cells) for use in immunotherapy. In some embodiments, the method is performed in vitro or ex vivo.

In some embodiments, the composition is introduced to a cell in a subject or to a cell in culture.

In some embodiments, the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPSc-derived T cell, invariant NKT cells (iNKT), iPSc-derived NKT, monocyte, or macrophage.

In some embodiments, the CRISPR nuclease and the RNA molecule are introduced to the cell at substantially the same time or at different times.

In some embodiments, alleles of the B2M gene in the cell are subjected to an insertion or deletion mutation.

In some embodiments, the insertion or deletion mutation creates an early stop codon.

In some embodiments, the inactivating results in a truncated protein encoded by the mutated allele.

In some embodiments, the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1538.

According to embodiments of the present invention, there is provided a method for inactivating alleles of the Beta-2 microglobulin (B2M) gene in a cell, the method comprising
introducing to the cell a composition comprising:
   at least one CRISPR nuclease, or a sequence encoding a CRISPR nuclease; and
   an RNA molecule comprising a guide sequence portion,
wherein a complex of the CRISPR nuclease and the RNA molecule affects a double strand break in alleles of the B2M gene,
wherein the guide sequence portion of the RNA molecule comprises 17-50 contiguous nucleotides containing nucleotides in the sequence set forth in any one of SEQ ID NOs: 1-1538 modified to contain 1, 2, 3, 4, or 5 nucleotide mismatches relative to a fully-complementary target sequence of the guide sequence portion.

In some embodiments, the guide sequence portion of the RNA molecule comprises 1, 2, 3, 4, or 5 nucleotide mismatches relative to a fully-complementary target sequence of the guide sequence portion.

In some embodiments, the guide sequence portion provides higher targeting specificity to the complex of the CRISPR nuclease and the RNA molecule relative to a guide sequence portion that has higher complementarity to an allele of the B2M gene.

In some embodiments, the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

According to embodiments of the present invention, there is provided a composition comprising an RNA molecule which comprises 17-50 contiguous nucleotides containing nucleotides in the sequence set forth in any one of SEQ ID NOs: 1-1538.

In some embodiments, the RNA molecule comprises 17-50 contiguous nucleotides containing nucleotides in the sequence set forth in any one of SEQ ID NOs: 75, 98, 136, 621, 685, 728, 747, 866, and 923.

In some embodiments, the RNA molecule comprises 17-50 contiguous nucleotides containing nucleotides in the sequence set forth in any one of SEQ ID NOs: 1-1538 other than SEQ ID NOs: 75, 98, 136, 621, 685, 728, 747, 866, and 923.

In some embodiments, the composition further comprises a second RNA molecule which comprises 17-50 contiguous nucleotides containing nucleotides in the sequence set forth in any one of SEQ ID NOs: 1-1538.

In some embodiments, the second RNA molecule which comprises 17-50 contiguous nucleotides containing nucleotides in the sequence set forth in any one of SEQ ID NOs: 75, 98, 136, 621, 685, 728, 747, 866, and 923.

In some embodiments, the second RNA molecule which comprises 17-50 contiguous nucleotides containing nucleotides in the sequence set forth in any one of SEQ ID NOs: 1-1538 is other than SEQ ID NOs: 75, 98, 136, 621, 685, 728, 747, 866, and 923.

According to embodiments of the present invention, there is provided a composition comprising an RNA molecule which comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

In some embodiments, the composition further comprises a second RNA molecule which comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

In some embodiments, any one of the compositions described above further comprises a CRISPR nuclease.

In some embodiments, any one of the compositions described above further comprises a tracrRNA molecule.

According to embodiments of the present invention, there is provided a cell modified by any one of the methods described herein or modified using the any one of the compositions described herein. The modified cell may also have additional genes altered in order to improve their use for adoptive transfer, for example, reducing or preventing graft-versus-host disease (GVHD).

In some embodiments, the cell is any one of a wherein the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPSc-derived T cell, invariant NKT cell (iNKT), iPS c-derived NKT, monocyte, or macrophage.

In some embodiments, the cell is a stem cell or any cell type capable of being reprogrammed to an induced pluripotent stem cell (iPSC).

In some embodiments, the stem cell is differentiated after it is modified.

In some embodiments, the stem cell is differentiated into any one of a lymphocyte, a T cell, a T regulatory cell, a B cell, a natural killer (NK) cell, innate-like T cell (iT), natural killer T cells (NKT), γδ T cell, invariant NKT cells (iNKT), monocyte, or macrophage.

According to embodiments of the present invention, there is provided a medicament comprising any one of the compositions described herein for use in inactivating a B2M allele in a cell, wherein the medicament is administered by delivering to the cell the composition.

According to embodiments of the present invention, there is provided a use of any one of the compositions or modified cells described herein for treating, ameliorating, or preventing host-versus-graft rejection (HvG), comprising delivering the composition or the modified cell to a subject experiencing or at risk of experiencing host-versus-graft rejection (HvG).

According to embodiments of the present invention, there is provided a medicament comprising any one of the compositions or modified cells described herein for use in treating, ameliorating, or preventing host-versus-graft rejection (HvG), wherein the medicament is administered by delivering the composition or the modified cell to a subject experiencing or at risk of experiencing host-versus-graft rejection (HvG).

According to embodiments of the present invention, there is provided a kit for inactivating a B2M allele in a cell, comprising any one of the compositions described herein and instructions for delivering the composition to the cell.

In some embodiments, the composition is delivered to the cell ex vivo.

According to embodiments of the present invention, there is provided a kit for treating or preventing host-versus-graft rejection (HvG) in a subject, comprising any one of the compositions or modified cells described herein and instructions for delivering the composition or modified cell to a subject experiencing or at risk of experiencing host-versus-graft rejection (HvG).

According to embodiments of the present invention, the modified immune cells (e.g., T cells) obtained by the described methods are intended to be used as a medicament for treating a cancer, infection, or immune disease in a subject in need thereof. The administration of the modified immune cell or a population thereof to a subject may be carried out in any convenient manner known in the art, including, but not limited to, aerosol inhalation, injection, ingestion, transfusion, implantation or transplantation. Injection or transfusion may be performed subcutaneously, intradermaliy, intratumorally, intranodally, intramedullary, intramuscularly, by intravenous or intralymphatic injection, or intraperitoneally. According to embodiments of the present invention, there is provided a method for adoptive cell therapy or prophylaxis comprising administering the modified cells to a subject suffering from or determined to be at risk of suffering from a cancer or an infection.

According to embodiments of the present invention, there is provided use of any one of the compositions or modified cells described herein for reducing or preventing host versus graft response (HvG) during treating, ameliorating, or preventing cancer, a disease, or an infection in a subject, comprising delivering the composition or modified cell to a subject experiencing or at risk of experiencing host-versus-graft rejection (HvG).

According to embodiments of the present invention, there is provided a medicament comprising any one of the modified cells described herein for treating, ameliorating, or preventing host-versus-graft rejection (HvG), such that the medicament is administered by delivering the composition to a subject experiencing or at risk of experiencing host-versus-graft rejection (HvG).

According to embodiments of the present invention, there is provided an RNA molecule for use in modifying a cell (e.g. lymphocyte, T-cell, CAR-T cell) to treat, ameliorate, or prevent host-versus-graft rejection (HvG) in a subject. The RNA molecule may be delivered to the cell ex vivo, in vitro, or in vivo.

According to embodiments of the present invention, there is provided a kit for inactivating a B2M allele in a cell, comprising any one of the compositions described herein and instructions for delivering the composition to the cell.

According to embodiments of the present invention, there is provided a kit for treating or preventing host-versus-graft rejection (HvG) in a subject, comprising any one of the compositions or modified cells described herein and instructions for delivering the composition or modified cell to a subject experiencing or at risk of experiencing host-versus-graft rejection (HvG).

According to embodiments of the present invention, there is provided a cell modified by the method described herein or modified using the compositions described herein.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-1538. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for inactivating B2M expression in a cell, the method comprising delivering to the cell a composition comprising an RNA molecule comprising a guide sequence portion having 17-50 contiguous nucleotides containing nucleotides in the sequence set forth in any one of SEQ ID NOs: 1-1538 and a CRISPR nuclease.

According to some embodiments of the present invention, there is provided a method for preventing host-versus-graft rejection (HvG), the method comprising delivering to a cell of a subject a composition comprising an RNA molecule comprising a guide sequence portion having 17-50 contiguous nucleotides containing nucleotides in the sequence set forth in any one of SEQ ID NOs: 1-1538 and a CRISPR nuclease.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

The compositions and methods of the present disclosure may be utilized for treating, preventing, ameliorating, or slowing progression of host-versus-graft rejection (HvG).

Any one of, or combination of, the above-mentioned strategies for deactivating B2M expression may be used in the context of the invention.

In embodiments of the present invention, an RNA molecule is used to direct a CRISPR nuclease to an exon or a splice site of a B2M allele in order to create a double-stranded break (DSB), leading to insertion or deletion of nucleotides by inducing an error-prone non-homologous end-joining (NHEJ) mechanism and formation of a frameshift mutation in the B2M allele. The frameshift mutation may result in, for example, inactivation or knockout of the B2M allele by generation of an early stop codon in the B2M allele and to generation of a truncated protein or to nonsense-mediated mRNA decay of the transcript of the allele. In further embodiments, one RNA molecule is used to direct a CRISPR nuclease to a promotor of a B2M allele.

In some embodiments, the method is utilized for treating a subject at risk for host-versus-graft rejection (HvG), which are disease phenotypes resulting from expression of the B2M gene. In such embodiments, the method results in improvement, amelioration, or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a lymphocyte. In some embodiments, the cell is a T cell. In some embodiments, the cell is a T regulatory cell. In some embodiments, the cell is a B cell. In some embodiments, the cell is a natural killer (NK) cell. In some embodiments, the cell is a macrophage. In some embodiments, the cell is a stem cell. In some embodiments, the cell is a fibroblast, blood cell, hepatocyte, keratinocyte, or any other cell type capable of being reprogrammed to an induced pluripotent stem cell (iPSC).

### B2M Editing Strategies

The present invention provides methods to knockout B2M alleles in cells of a subject, thereby avoiding host-versus-graft rejection (HvG) upon adoptive transfer of the cells or cells derived therefrom to a subject in need of the adoptive transfer. The provided methods to knockout B2M alleles in a cell may be used to treat, prevent, or ameliorate host-versus-graft rejection (HvG).

B2M editing strategies include, but are not limited to, biallelic knockout by targeting any one of, or a combination of, Exons 1-3, including within seven nucleotides upstream and downstream of the exons to flank splice donor and acceptor sites, as frameshifts in these exons lead to non-functional, truncated B2M proteins or non-sense mediated decay of the mutated B2M transcripts.

### CRISPR Nucleases and PAM Recognition

In some embodiments, the sequence specific nuclease is selected from CRISPR nucleases, or is a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to all B2M alleles in a cell. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8al, Cas8a2, Cas8b, Cas8c, Cas9, Casl 0, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-O-methyl (M), 3′-phosphorothioate (MS), 3′-thioPACE (MSP), pseudouridine, or 1-methyl pseudo-uridine. Each possibility represents a separate embodiment of the present invention.

In addition to targeting B2M alleles by a RNA-guided CRISPR nuclease, other means of inhibiting B2M expression in a target cell include but are not limited to use of a gapmer, shRNA, siRNA, a customized TALEN, meganuclease, or zinc finger nuclease, a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736; WO/2006/097853; WO/2003/087341; WO/2000/0415661; WO/2003/080809; WO/2010/079430; WO/2010/079430; WO/2011/072246; WO/2018/057989; and WO/2017/164230, the entire contents of each of which are incorporated herein by reference.

Advantageously, the guide RNA molecules presented herein provide improved B2M knockout efficiency when complexed with a CRISPR nuclease in a cell relative to other guide RNA molecules. These specifically designed sequences may also be useful for identifying B2M target sites for other nucleotide targeting-based gene-editing or gene-silencing methods, for example, siRNA, TALENs, meganucleases or zinc-finger nucleases.

### Delivery to Cells

Any one of the compositions described herein may be delivered to a target cell by any suitable means. RNA molecule compositions of the present invention may be targeted to any cell which contains and/or expresses a B2M allele, such as a mammalian lymphocyte or stem cell. For example, in one embodiment the RNA molecule specifically targets B2M alleles in a target cell and the target cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC). The delivery to the cell may be performed in vivo, ex vivo, or in vitro. Further, the nucleic acid compositions described herein may be delivered to a cell as one or more of DNA molecules, RNA molecules, ribonucleoproteins (RNP), nucleic acid vectors, or any combination thereof.

In some embodiments, the RNA molecule comprises a chemical modification. Non-limiting examples of suitable chemical modifications include 2′-O-methyl (M). 2′-O-methyl, 3′phosphorothioate (MS) or 2′-O-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

Examples of RNA Guide Sequences which Specifically Target Alleles of B2M Gene

Although a large number of guide sequences can be designed to target the B2M gene, the nucleotide sequences described in Table 1 and are identified by SEQ ID NOs: 1-1538 were specifically selected to effectively implement the methods set forth herein.

Table 1 shows guide sequences designed for use as described in the embodiments above to associate with target sequences of the B2M gene. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

As used herein, the following nucleotide identifiers are used to represent a referenced nucleotide base(s):

| Nucleotide | | | | |
|---|---|---|---|---|
| reference | Base(s) represented | | | |
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

TABLE 1
Guide sequences designed to associate with specific B2M gene targets

| | SEQ ID NOs: of | SEQ ID NOs: of | SEQ ID NOs: of |
|---|---|---|---|
| Target | 20 base guides | 21 base guides | 22 base guides |
| 15:44711539-15:44711620 | 1-64 | 65-155 | 156-253 |
| Exon 1, including the ATG start | | | |
| codon to 7nt DS of Exon 1 | | | |
| 15:44715415-15:44715708 | 254-595 | 596-1024 | 1025-1516 |
| Exon 2, including 7nt US and | | | |
| 7nt DS to Exon 2 | | | |
| 15:44716321-15:44716349 | 1517-1520 | 1521-1524 | 1525-1528 |
| Exon 3, including 7nt US of | | | |
| Exon 3 to the stop codon | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: Dec. 2013 initial release; Dec. 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: B2M Modification Analysis

Guide sequences comprising 17-50 contiguous nucleotides containing nucleotides in the sequence set forth in any one of SEQ ID NOs: 1-1538 are screened for high on target activity using SpCas9 in T cells. On target activity is determined by DNA capillary electrophoresis analysis.

Additional examples demonstrating the editing ability of B2M specific RNA guides are described below and may also be found in PCT International Publication No. WO 2020/223514 A2, the contents of which are hereby incorporated by reference.

### Example 2: OMNI-50 CRISPR Nuclease RNP Activity Using B2M Guides

B2M Editing by an OMNI-50 Nuclease was Observed in Primary T Cells, as can be seen in Table 2. 57% of the B2M guides were active, with editing levels ranging between 5% and 61%. Considering the potential further optimization, full knock-out B2M by OMNI-50 is possible with the appropriate strategy.

TABLE 2
OMNI-50 Activity as an RNP
OMNI-50 RNP was assembled with synthetic sgRNA (Agilent) and electroporated
into cells. Several cell types were tested with a variety of sgRNAs.
Cellular system, gene name, and spacer sequences are indicated next to
the editing level as measured by NGS.

| | Genomic | Corresponding | | % |
|---|---|---|---|---|
| System | site | spacer name | Spacer sequence | indels |
| Primary T | B2M | gRNA 1 | UACUCUCUCUUUCUGGCCUGG | 5% |
| cells | | | (SEQ ID NO: 136) | |
| | | gRNA 2 | GCAUACUCAUCUUUUUCAGUG | 12% |
| | | | (SEQ ID NO: 1529) | |
| | | gRNA 3 | CGCUACUCUCUCUUUCUGGCC | 17% |
| | | | (SEQ ID NO: 98) | |
| | | gRNA 4 | GCGCGAGCACAGCUAAGGCCA | 64% |
| | | | (SEQ ID NO: 1530) | |
| | | gRNA 6 | GCUCGCGCUACUCUCUCUUUC | 9% |
| | | | (SEQ ID NO: 1531) | |
| | | gRNA 7 | AGAGUAGCGCGAGCACAGCUA | 61% |
| | | | (SEQ ID NO: 75) | |
| | | gRNA 15 | UCACAGCCCAAGAUAGUUAAG | 45% |
| | | | (SEQ ID NO: 1532) | |
| | | gRNA 16 | CACAGCCCAAGAUAGUUAAGU | 42% |
| | | | (SEQ ID NO: 728) | |
| | | gRNA 18 | GACAAAGUCACAUGGUUCACA | 43% |
| | | | (SEQ ID NO: 1533) | |
| | | gRNA 19 | AAGUCACAUGGUUCACACGGC | 8% |
| | | | (SEQ ID NO: 621) | |
| | | gRNA 20 | AGGCAUACUCAUCUUUUUCAG | 37% |
| | | | (SEQ ID NO: 685) | |
| | | gRNA 21 | GGCAUACUCAUCUUUUUCAGU | 33% |
| | | | (SEQ ID NO: 866) | |
| | | gRNA 22 | CAUACUCAUCUUUUUCAGUGG | 29% |
| | | | (SEQ ID NO: 747) | |
| | | gRNA 23 | UCAGUAAGUCAACUUCAAUGU | 41% |
| | | | (SEQ ID NO: 923) | |
| | | gRNA 26 | ACGUGAGUAAACCUGAAUCUU | 22% |
| | | | (SEQ ID NO: 1534) | |

### Example 3: Multiplexing B2M and TRAC Editing

B2M and TRAC genes may be knocked out simultaneously by using a multiplexing approach. B2M and TRAC multiplex editing was tested by mixing two RNP populations and electroporating the mix into primary T cells. gRNA 32 was used for TRAC targeting and gRNA 15 was used for B2M targeting (spacer sequences are listed in Table 3). At 72 h cells were harvested and tested for editing by NGS. The TRAC gene measured 50% editing, and the B2M gene measured 25% editing. These results were similar to editing levels with a single RNP that was performed side-by-side to the multiplex test (Table 3).

TABLE 3
OMNI-50 Multiplexing
OMNI-50 multiplexing in primary T cells: Multiplexing of OMNI-50 was
performed by electroporation into activated primary T cells, targeting
either B2M or TRAC genes, or combined targeting. The first two rows show
each gene separately on two donors tha twere randomly chosen from a five-
donor bank. The final two rows show the same analysis for each gene when
electroporation was performed as a multiplex. Editing activity was
determined by indel count after amplicon based NGS. Standard deviation
of duplicates is also shown. Using only B2M gRNA had no effect on the
TRAC gene and vice versa (not shown).

| | | | OMNI-50 Editing | | OMNI-50 STD | |
|---|---|---|---|---|---|---|
| Gene | Site | Spacer Sequence | Donor 1 | Donor 2 | Donor 1 | Donor 2 |
| TRAC | gRNA 32 | GCCACUUUCAG | 59.00 | 53.00 | 9.00 | 10.00 |
| | | GAGGAGGAUU | | | | |
| | | (SEQ ID NO: | | | | |
| | | 1544) | | | | |
| B2M | gRNA 15 | UCACAGCCCAA | 42.00 | 44.00 | 13.00 | 19.00 |
| | | GAUAGUUAAG | | | | |
| | | (SEQ ID NO: | | | | |
| | | 1532) | | | | |
| TRAC + | gRNA 32 + | Test for TRAC | 55.00 | 44.00 | 5.00 | 1.00 |
| B2M | gRNA 15 | | | | | |
| TRAC + | gRNA 32 + | Test for B2M | 22.00 | 27.00 | 3.00 | 1.00 |
| B2M | gRNA 15 | | | | | |

### Example 4: Additional OMNI CRISPR Nuclease Activity Assays Using B2M Specific Guides

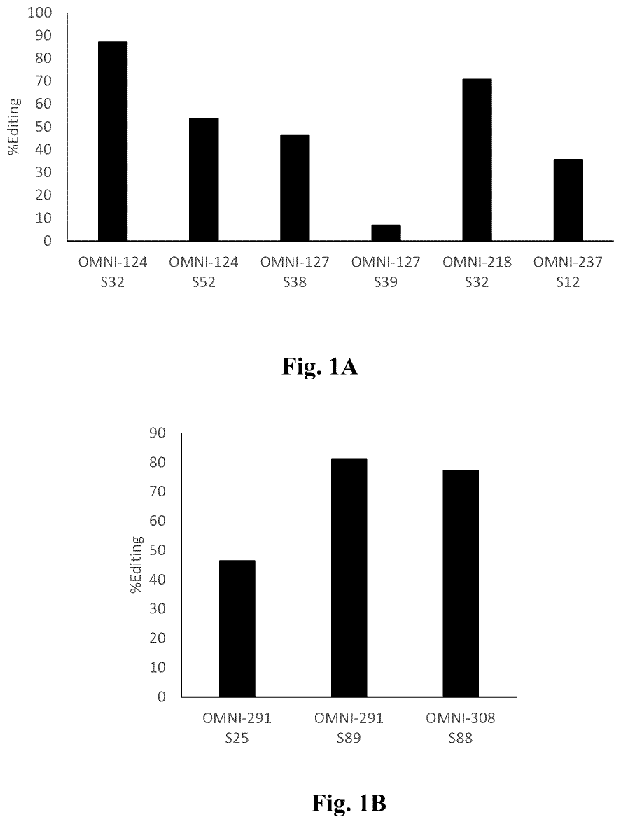

The B2M gene encodes a protein found in association with the major histocompatibility complex (MHC) class I heavy chain on the surface of nearly all nucleated cells. In HeLa cells, the CRIPSR nucleases listed in Table 4 were screened by introducing a nuclease and guide RNA molecule (Table 5) to a cell. Briefly, a screen was performed in 96-well format with 64 ng nuclease co-transfected with each of the guides at 20 ng using JetOPTIMUS reagent (Polyplus). Cells were harvested 72 hours post DNA transfection. Cell lysis and genomic DNA extraction was performed using Quick Extract (Lucigen) and endogenous genomic regions were amplified using specific primers to measure editing activity by next-generation sequencing (NGS). The screen identified six (6) active novel nucleases. Editing was observed in nine (9) sites along the B2M gene (FIGS. 1A and 1B, which lists the respective nuclease sequences (Table 4) and the spacer sequences (Table 5) in the x-axis). The sites for editing were chosen according to the PAM requirements for each nuclease, which was obtained by in vitro TXTL assay.

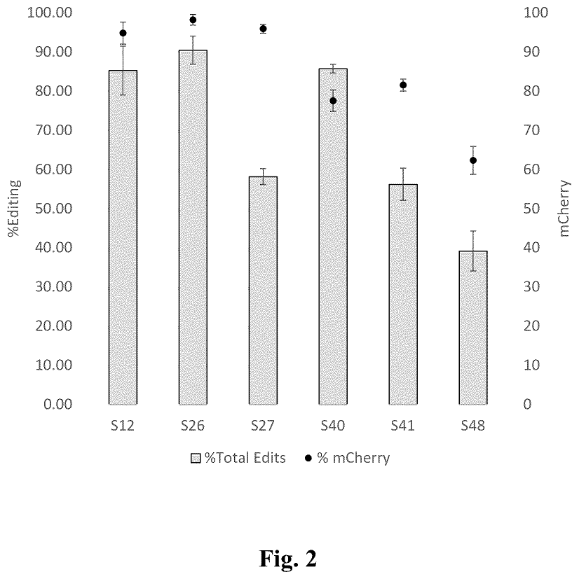

OMNI-103 CRISPR nuclease was further screened in HeLa cells for additional sites along the B2M gene. To this end, the corresponding OMNI-P2A-mCherry expression vector (Table 6) was transfected into HeLa cells together with a sgRNA molecule designed to target specific location in the B2M gene (gRNA or “spacer” sequences are listed in Table 5). At 72 h, cells were harvested, and half of the cells were used for quantification of transfection efficiency by FACS using mCherry fluorescence as marker. The other half of the cells were lysed, and their genomic DNA content was used in PCR reaction that amplified the corresponding putative genomic targets. Amplicons were subjected to NGS and the resulting sequences were then used to calculate the percentage of editing events in each target site. Short insertions or deletions (indels) around the cut site are the typical outcome of repair of DNA ends following nuclease induced DNA cleavage. The calculation of % editing was therefore deduced from the fraction of indel-containing sequences within each amplicon. Significant editing activity was observed in six (6) sites along the B2M gene (FIG. 2).

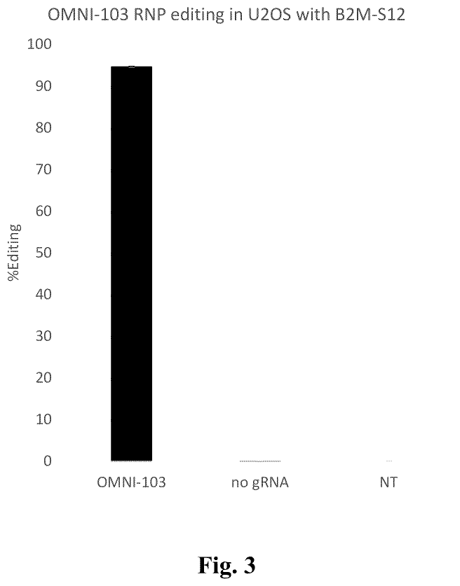

OMNI-103 was further developed for use as part of an RNP and tested in a U2OS cell line complexed with a synthetic gRNA comprising a guide sequence portion targeting site 12 (S12), which showed high editing in the HeLa DNA screen. Briefly, 2×10 5 U2OS cells were mixed with preassembled RNPs composed of 105 pmole OMNI-103 protein and 120 pmole of a sgRNA comprising a 22-nucleotide guide sequence portion mixed with 100 pmole of electroporation enhancer (IDT-1075916). The mixture was electroporated into the cells using SE cell 4D-nucleofector X Kit S (V4XP-3032, Lonza) by applying the DN-100 program. OMNI-103 with no gRNA molecule served as a negative control. A fraction of cells were harvested 72 h post electroporation and genomic DNA was extracted to measure on-target activity by NGS. According to NGS analysis, in this assay indel formation was above 90% as well (FIG. 3).

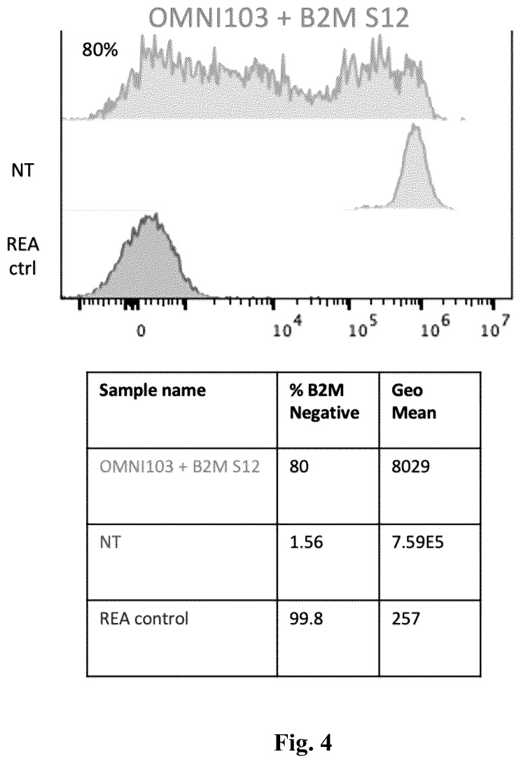

OMNI-103 was further tested as part of an RNP for B2M editing in primary T cells using the same gRNA molecules showing editing activity in the U2OS cell system. Briefly, primary T cells were isolated from blood donations (Israel blood bank) using the buffy coat method for PBMC isolation, followed by negative isolation of CD3+ cells (T cell isolation kit, 130-096-535 Miltenyi). Primary T cells were then activated by CD3 and CD28 beads (T cell activation/expansion kit, 130-091-441 Miltenyi). 2×10⁵ primary T cells were mixed with preassembled RNPs composed of 113 pmole OMNI-103 protein and 160 pmole of a sgRNA having a 22-nucleotide guides sequence portion, and electroporated using P3 cell 4D-nucleofector X Kit S (V4XP-3032, Lonza) by applying the EH-115 program. A fraction of the cells were harvested 72 h post electroporation and B2M surface expression was measured by flow cytometry (anti-B2M APC, 1:100, #316312 Biolegend). According to FACS analysis, editing activity measured 68% (FIG. 4).

TABLE 4
OMNI CRISPR Nuclease Elements

| | SEQ ID NO | SEQ ID NO | SEQ ID NO of | |
|---|---|---|---|---|
| | of OMNI | of DNA | DNA sequence | |
| | Amino | sequence | codon optimized | |
| OMNI CRISPR | Acid | encoding | for expression in | OMNI sgRNA |
| Nuclease Name | Sequence | OMNI | human cells | Scaffold Sequence |
| OMNI-103 | 1545 | 1552 | 1559 | 1566 |
| OMNI-124 | 1546 | 1553 | 1560 | 1567 |
| OMNI-127 | 1547 | 1554 | 1561 | 1568 |
| OMNI-218 | 1548 | 1555 | 1562 | 1569 |
| OMNI-237 | 1549 | 1556 | 1563 | 1570 |
| OMNI-291 | 1550 | 1557 | 1564 | 1571 |
| OMNI-308 | 1551 | 1558 | 1565 | 1572 |

TABLE 5
Guide Sequence Portion (“Spacer”) Elements

| Spacer | | PAM Site | Compatible OMNI |
|---|---|---|---|
| Name | Spacer Sequence | Utilized | CRISPR Nucleases |
| B2M-S12 | GUAUGCCUGCCGUGUGAACCAU | GTGACTTT | OMNI-103, OMNI-237 |
| | (SEQ ID NO: 1347) | | |
| B2M-S25 | UUUCAUCCAUCCGACAUUGAAG | TTGACTTA | OMNI-291 |
| | (SEQ ID NO: 1507) | | |
| B2M-S26 | UCUCUCUCCAUUCUUCAGUAAG | TCAACTTC | OMNI-103 |
| | (SEQ ID NO: 1416) | | |
| B2M-S27 | AGAAUUGAAAAAGUGGAGCAUU | CAGACTTG | OMNI-103 |
| | (SEQ ID NO: 1104) | | |
| B2M-S32 | AGCGCGAGCACAGCUAAGGCCA | CGGAGCGA | OMNI-124, OMNI-218 |
| | (SEQ ID NO: 170) | | |
| B2M-S40 | AAGAAUGUAAGACUUACCCCAC | TTAACTAT | OMNI-103 |
| | (SEQ ID NO: 1535) | | |
| B2M-S41 | UCAGCAGCUUACAAAAGAAUGU | AAGACTTA | OMNI-103 |
| | (SEQ ID NO: 1536) | | |
| B2M-S48 | CGUCGCGCUGGCGGGCAUUCCU | GAAGCTGA | OMNI-103 |
| | (SEQ ID NO: 1537) | | |
| B2M-S52 | CGUGAGUAAACCUGAAUCUUUG | GAGTACCT | OMNI-124 |
| | (SEQ ID NO: 1231) | | |
| B2M-S38 | CUCUUUCUGGCCUGGAGGCUAU | CCAGCGTG | OMNI-127 |
| | (SEQ ID NO: 197) | | |
| B2M-S39 | GAUUCAGGUUUACUCACGUCAU | CCAGCAGA | OMNI-127 |
| | (SEQ ID NO: 1309) | | |
| B2M-S88 | GCUGGCGGGCAUUCCUGAAGCU | GACAGCAT | OMNI-308 |
| | (SEQ ID NO: 1538) | | |
| B2M-S89 | CAAAGUCACAUGGUUCACACGG | CAGGCATA | OMNI-291 |
| | (SEQ ID NO: 1167) | | |

TABLE 6
Plasmid Constructs and Elements

| | | Coding ORF | | |
|---|---|---|---|---|
| Plasmid | Elements | Sequence | Example | DNA Sequence |
| pET9a | Expressing OMNI | T7 promoter HA Tag- | pET9a-OMNI-103 | SEQ ID NO: 1573 |
| | polypeptide in the | Linker-OMNI ORF | | |
| | bacterial system | (Human optimized)- | | |
| | | SV40 NLS-8XHisTag- | | |
| | | T7 terminator | | |
| pbShuttle | Expressing OMNI | U6 promotor- | pShuttle Guide-T2- | SEQ ID NO: 1574 |
| Guide T2 | sgRNA in the | T7promoter-T2 | OMNI-103 V2 | |
| | bacterial system | spacer sgRNA | | |
| | | scaffold-T7 | | |
| | | terminator | | |
| pbPOS T2 | Bacterial/TXTL | T2 protospacer-8N | pbPOS T2 library | SEQ ID NO: 1575 |
| library | depletion assay | PAM library- | | |
| | | chloramphenicol | | |
| | | acetyltransferase | | |

### Example 5: Editing in Primary Natural Killer Cells

Aim: Calibrate electroporation in primary NK with OMNI-103 and B2M-S12.

Procedure: Cells were isolated from an apheresis sample. Cells were accepted with feeders and NK percentage was measured to be >82%. There were 5×10⁵ cells per well and the electroporation program was tested with OMNI-103 (113 μmol) tag free (Phenyl) sd60082 and an sgRNA comprising a B2M-S12 guide sequence portion (Agilent). After 72 hours half the number of cells were measured using FACS. The gating strategy was as follows: lymphocytes were gated on singlets and then on live cells (DAPI stain). From the live cells, the CD56 positive cells (˜95%) were gated first, followed by the B2M negative cells.

TABLE 7
Results-

| Sample | Nuclease | Guide Sequence Portion | % Editing |
|---|---|---|---|
| Primary T cells treated | OMNI-103 | GUAUGCCUGCCGUGUGAAC | 50.3% |
| with OMNI-103 + | | CAU (SEQ ID NO: 1347) | |
| guide B2M site 12 | | | |
| Negative control: non- | No nuclease | No guide | 0.2% |
| treated primary T cells | | | |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genomme editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1995) “Transfer of genes to humans: early lessons and obstacles to success”, Science 270(5235):404-10.
15. Dillon (1993) “Regulation gene expression in gene therapy” Trends in Biotechnology 11(5):167-173.
16. Dranoff et al. (1997) “A phase I study of vaccination with autologous, irradiated melanoma cells engineered to secrete human granulocyte macrophage colony stimulating factor”, Hum. Gene Ther. 8(1):111-23.
17. Dunbar et al. (1995) “Retrovirally marked CD34-enriched peripheral blood and bone marrow cells contribute to long-term engraftment after autologous transplantation”, Blood 85:3048-57.
18. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
19. Gao and Huang (1995) “Cationic liposome-mediated gene transfer” Gene Ther. 2(10):710-22.
20. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
21. Han et al. (1995) “Ligand-directed retro-viral targeting of human breast cancer cells”, Proc Natl Acad Sci U.S.A. 92(21):9747-51.
22. Hu et al. (2018) “Evolved Cas9 variants with broad PAM compatibility and high DNA specificity”, Nature 556(7699):57-63.
23. Jinek et al. (2012) “A programmable dual-RNA-guided DNA endonuclease in adaptive bacterial immunity,” Science 337(6096):816-21.
24. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
25. Judge et al. (2006) “Design of noninflammatory synthetic siRNA mediating potent gene silencing in vivo”, Mol Ther. 13(3):494-505.
26. Kohn et al. (1995) “Engraftment of gene-modified umbilical cord blood cells in neonates with adnosine deaminase deficiency”, Nature Medicine 1:1017-23.
27. Kremer and Perricaudet (1995) “Adenovirus and adeno-associated virus mediated gene transfer”, Br. Med. Bull. 51(1):31-44.
28. Macdiarmid et al. (2009) “Sequential treatment of drug-resistant tumors with targeted minicells containing siRNA or a cytotoxic drug”, Nat Biotehcnol. 27(7):643-51.
29. Malech et al. (1997) “Prolonged production of NADPH oxidase-corrected granulocyes after gene therapy of chronic granulomatous disease”, PNAS 94(22):12133-38.
30. Miller et al. (1991) “Construction and properties of retrovirus packaging cells based on gibbon ape leukemia virus”, J Virol. 65(5):2220-24.
31. Miller (1992) “Human gene therapy comes of age”, Nature 357:455-60.
32. Mitani and Caskey (1993) “Delivering therapeutic genes—matching approach and application”, Trends in Biotechnology 11(5):162-66.
33. Nabel and Felgner (1993) “Direct gene transfer for immunotherapy and immunization”, Trends in Biotechnology 11(5):211-15.
34. Nishimasu et al. (2018) “Engineered CRISPR-Cas9 nuclease with expanded targeting space”, Science 361(6408):1259-1262.
35. Remy et al. (1994) “Gene Transfer with a Series of Lipphilic DNA-Binding Molecules”, Bioconjugate Chem. 5(6):647-54.
36. Sentmanat et al. (2018) “A Survey of Validation Strategies for CRISPR-Cas9 Editing”, Scientific Reports 8:888, doi:10.1038/s41598-018-19441-8.
37. Sommerfelt et al. (1990) “Localization of the receptor gene for type D simian retroviruses on human chromosome 19”, J. Virol. 64(12):6214-20.
38. Van Brunt (1988) “Molecular framing: transgenic animals as bioactors” Biotechnology 6:1149-54.
39. Vigne et al. (1995) “Third-generation adenovectors for gene therapy”, Restorative Neurology and Neuroscience 8(1,2): 35-36.
40. Walton et al. (2020) “Unconstrained genome targeting with near-PAMless engineered CRISPR-Cas9 variants”, Science 368(6488):290-296.
41. Weissman and Kariko (2015) “mRNA:Fulfilling the promise of gene therapy”, Molecular Therapy (9):1416-7.
42. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
43. Yu et al. (1994) “Progress towards gene therapy for HIV infection”, Gene Ther. 1(1):13-26.
44. Zetsche et al. (2015) “Cpf1 is a single RNA-guided endonuclease of a class 2 CRIPSR-Cas system” Cell 163(3):759-71.
45. Zuris et al. (2015) “Cationic lipid-mediated delivery of proteins enables efficient protein based genome editing in vitro and in vivo” Nat Biotechnol. 33(1):73-80.

1. A method for inactivating alleles of the Beta-2 microglobulin (B2M) gene in a cell, the method comprising
introducing to the cell a composition comprising:
at least one CRISPR nuclease, or a sequence encoding a CRISPR nuclease; and
an RNA molecule comprising a guide sequence portion,
wherein a complex of the CRISPR nuclease and the RNA molecule affects a double strand break in alleles of the B2M gene,
wherein the guide sequence portion of the RNA molecule comprises 17-50 contiguous nucleotides containing nucleotides in the sequence set forth in any one of SEQ ID NOs: 1-1538.

2. The method of claim 1, wherein the composition is introduced to a cell in a subject or to a cell in culture.

3. The method of any one of claims 1-2, wherein the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

4. The method of any one of claims 1-2, wherein the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPS c-derived T cell, invariant NKT cells (iNKT), iPSc-derived NKT, monocyte, or macrophage.

5. The method of any one of claims 1-4, wherein the CRISPR nuclease and the RNA molecule are introduced to the cell at substantially the same time or at different times.

6. The method of any one of claims 1-5, wherein alleles of the B2M gene in the cell are subjected to an insertion or deletion mutation.

7. The method of claim 6, wherein the insertion or deletion mutation creates an early stop codon.

8. The method of any one of claims 1-7, wherein the inactivating results in a truncated protein encoded by the mutated allele.

9. The method of any one of claims 1-8, wherein the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1538.

10. A method for inactivating alleles of the Beta-2 microglobulin (B2M) gene in a cell, the method comprising
introducing to the cell a composition comprising:
at least one CRISPR nuclease, or a sequence encoding a CRISPR nuclease; and
an RNA molecule comprising a guide sequence portion,
wherein a complex of the CRISPR nuclease and the RNA molecule affects a double strand break in alleles of the B2M gene,
wherein the guide sequence portion of the RNA molecule comprises 17-50 contiguous nucleotides containing nucleotides in the sequence set forth in any one of SEQ ID NOs: 1-1538 modified to contain 1, 2, 3, 4, or 5 nucleotide mismatches relative to a fully-complementary target sequence of the guide sequence portion.

11. The method of claim 10, wherein the guide sequence portion of the RNA molecule comprises 1, 2, 3, 4, or 5 nucleotide mismatches relative to a fully-complementary target sequence of the guide sequence portion.

12. The method of any one of claim 10 or 11, wherein the guide sequence portion provides higher targeting specificity to the complex of the CRISPR nuclease and the RNA molecule relative to a guide sequence portion that has higher complementarity to an allele of the B2M gene.

13. The method of any one of claims 10-12, wherein the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

14. A composition comprising an RNA molecule which comprises 17-50 contiguous nucleotides containing nucleotides in the sequence set forth in any one of SEQ ID NOs: 1-1538.

15. The composition of claim 14, wherein the RNA molecule comprises 17-50 contiguous nucleotides containing nucleotides in the sequence set forth in any one of SEQ ID NOs: 75, 98, 136, 621, 685, 728, 747, 866, and 923.

16. The composition of claim 14, wherein the RNA molecule comprises 17-50 contiguous nucleotides containing nucleotides in the sequence set forth in any one of SEQ ID NOs: 1-1538 other than SEQ ID NOs: 75, 98, 136, 621, 685, 728, 747, 866, and 923.

17. The composition of any one of claims 14-16, further comprising a second RNA molecule which comprises 17-50 contiguous nucleotides containing nucleotides in the sequence set forth in any one of SEQ ID NOs: 1-1538.

18. The composition of claim 17, wherein the second RNA molecule which comprises 17-50 contiguous nucleotides containing nucleotides in the sequence set forth in any one of SEQ ID NOs: 75, 98, 136, 621, 685, 728, 747, 866, and 923.

19. The composition of claim 17, wherein the second RNA molecule which comprises 17-50 contiguous nucleotides containing nucleotides in the sequence set forth in any one of SEQ ID NOs: 1-1538 is other than SEQ ID NOs: 75, 98, 136, 621, 685, 728, 747, 866, and 923.

20. A composition comprising an RNA molecule which comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

21. The composition of claim 20, further comprising a second RNA molecule which comprises 17-50 contiguous nucleotides containing nucleotides in the sequence set forth in any one of SEQ ID NOs: 1-1538, or any one of SEQ ID NOs: 1-1538 modified to contain 1, 2, 3, 4, or 5 nucleotide mismatches relative to a fully-complementary target sequence of the guide sequence portion.

22. The composition of any one of claims 14-21, further comprising a CRISPR nuclease.

23. The composition of any one of claims 14-22, further comprising a tracrRNA molecule.

24. A cell modified by the method of any one of claims 1-13 or modified using the composition of any one of claims 14-23.

25. The modified cell of claim 24, wherein the cell is any one of a wherein the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

26. The modified cell of claim 24, wherein the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPSc-derived T cell, invariant NKT cell (iNKT), iPSc-derived NKT, monocyte, or macrophage.

27. The modified cell of claim 24, wherein the cell is a stem cell or any cell type capable of being reprogrammed to an induced pluripotent stem cell (iPSC).

28. The modified cell of claim 27, wherein the stem cell is differentiated after it is modified.

29. The modified cell of claim 28, wherein the stem cell is differentiated into any one of a lymphocyte, a T cell, a T regulatory cell, a B cell, a natural killer (NK) cell, innate-like T cell (iT), natural killer T cells (NKT), γδ T cell, invariant NKT cells (iNKT), monocyte, or macrophage.

30. A medicament comprising the composition of any one of claims 14-23 for use in inactivating a B2M allele in a cell, wherein the medicament is administered by delivering to the cell the composition of any one of claims 14-23.

31. Use of the composition of any one of claims 14-23 or the modified cell of any one of claims 24-29 for treating, ameliorating, or preventing host-versus-graft rejection (HvG), comprising delivering the composition of any one of claims 14-23 or the modified cell of any one of claims 24-29 to a subject experiencing or at risk of experiencing host-versus-graft rejection (HvG).

32. A medicament comprising the composition of any one of claims 14-23 or the modified cell of any one of claims 24-29 for use in treating, ameliorating, or preventing host-versus-graft rejection (HvG), wherein the medicament is administered by delivering the composition of any one of claims 14-23 or the modified cell of any one of claims 24-29 to a subject experiencing or at risk of experiencing host-versus-graft rejection (HvG).

33. A kit for inactivating a B2M allele in a cell, comprising the composition of any one of claims 14-23 and instructions for delivering the composition to the cell.

34. The kit of claim 33, wherein the composition is delivered to the cell ex vivo.

35. A kit for treating or preventing host-versus-graft rejection (HvG) in a subject, comprising the composition of any one of claims 14-23 or the modified cell of any one of claims 24-29 and instructions for delivering the composition or modified cell to a subject experiencing or at risk of experiencing host-versus-graft rejection (HvG).


SEQUENCE LISTING (1583 sequences)

SEQ ID NO: 1 - RNA, 20 nt, Artificial Sequence
  B2M-targeting guide sequence portion
aaagagagaguagcgcgagc

SEQ ID NO: 2 - RNA, 20 nt, Artificial Sequence
  B2M-targeting guide sequence portion
aaggccacggagcgagacau

SEQ ID NO: 3 - RNA, 20 nt, Artificial Sequence
  B2M-targeting guide sequence portion
agaaagagagaguagcgcga

SEQ ID NO: 4 - RNA, 20 nt, Artificial Sequence
  B2M-targeting guide sequence portion
agagagaguagcgcgagcac

SEQ ID NO: 5 - RNA, 20 nt, Artificial Sequence
  B2M-targeting guide sequence portion
agagaguagcgcgagcacag

SEQ ID NO: 6 - RNA, 20 nt, Artificial Sequence
  B2M-targeting guide sequence portion
agaugucucgcuccguggcc

SEQ ID NO: 7 - RNA, 20 nt, Artificial Sequence
  B2M-targeting guide sequence portion
agcacagcuaaggccacgga

SEQ ID NO: 8 - RNA, 20 nt, Artificial Sequence
  B2M-targeting guide sequence portion
agccuccaggccagaaagag

SEQ ID NO: 9 - RNA, 20 nt, Artificial Sequence
  B2M-targeting guide sequence portion
agcgcgagcacagcuaaggc

SEQ ID NO: 10 - RNA, 20 nt, Artificial Sequence
  B2M-targeting guide sequence portion
agcuaaggccacggagcgag

SEQ ID NO: 11 - RNA, 20 nt, Artificial Sequence
  B2M-targeting guide sequence portion
aggccagaaagagagaguag

SEQ ID NO: 12 - RNA, 20 nt, Artificial Sequence
  B2M-targeting guide sequence portion
aguagcgcgagcacagcuaa

SEQ ID NO: 13 - RNA, 20 nt, Artificial Sequence
  B2M-targeting guide sequence portion
auagccuccaggccagaaag

SEQ ID NO: 14 - RNA, 20 nt, Artificial Sequence
  B2M-targeting guide sequence portion
augucucgcuccguggccuu

SEQ ID NO: 15 - RNA, 20 nt, Artificial Sequence
  B2M-targeting guide sequence portion
cagaaagagagaguagcgcg

SEQ ID NO: 16 - RNA, 20 nt, Artificial Sequence
  B2M-targeting guide sequence portion
cagcuaaggccacggagcga

SEQ ID NO: 17 - RNA, 20 nt, Artificial Sequence
  B2M-targeting guide sequence portion
ccaggccagaaagagagagu

SEQ ID NO: 18 - RNA, 20 nt, Artificial Sequence
  B2M-targeting guide sequence portion
ccgagaugucucgcuccgug

SEQ ID NO: 19 - RNA, 20 nt, Artificial Sequence
  B2M-targeting guide sequence portion
ccuccaggccagaaagagag

SEQ ID NO: 20 - RNA, 20 nt, Artificial Sequence
  B2M-targeting guide sequence portion
ccuuagcugugcucgcgcua

SEQ ID NO: 21 - RNA, 20 nt, Artificial Sequence
  B2M-targeting guide sequence portion
cgagaugucucgcuccgugg

SEQ ID NO: 22 - RNA, 20 nt, Artificial Sequence
  B2M-targeting guide sequence portion
cgcgcuacucucucuuucug

SEQ ID NO: 23 - RNA, 20 nt, Artificial Sequence
  B2M-targeting guide sequence portion
cgcuccguggccuuagcugu

SEQ ID NO: 24 - RNA, 20 nt, Artificial Sequence
  B2M-targeting guide sequence portion
cgcuggauagccuccaggcc

SEQ ID NO: 25 - RNA, 20 nt, Artificial Sequence
  B2M-targeting guide sequence portion
cuaaggccacggagcgagac

SEQ ID NO: 26 - RNA, 20 nt, Artificial Sequence
  B2M-targeting guide sequence portion
cuacucucucuuucuggccu

SEQ ID NO: 27 - RNA, 20 nt, Artificial Sequence
  B2M-targeting guide sequence portion
cucacgcuggauagccucca

SEQ ID NO: 28 - RNA, 20 nt, Artificial Sequence
  B2M-targeting guide sequence portion
cuccaggccagaaagagaga

SEQ ID NO: 29 - RNA, 20 nt, Artificial Sequence
  B2M-targeting guide sequence portion
cuccguggccuuagcugugc

SEQ ID NO: 30 - RNA, 20 nt, Artificial Sequence
  B2M-targeting guide sequence portion
cucuuucuggccuggaggcu

SEQ ID NO: 31 - RNA, 20 nt, Artificial Sequence
  B2M-targeting guide sequence portion
cuggauagccuccaggccag

SEQ ID NO: 32 - RNA, 20 nt, Artificial Sequence
  B2M-targeting guide sequence portion
cugugcucgcgcuacucucu

SEQ ID NO: 33 - RNA, 20 nt, Artificial Sequence
  B2M-targeting guide sequence portion
cuuagcugugcucgcgcuac

SEQ ID NO: 34 - RNA, 20 nt, Artificial Sequence
  B2M-targeting guide sequence portion
cuuucuggccuggaggcuau

SEQ ID NO: 35 - RNA, 20 nt, Artificial Sequence
  B2M-targeting guide sequence portion
gaaagagagaguagcgcgag

SEQ ID NO: 36 - RNA, 20 nt, Artificial Sequence
  B2M-targeting guide sequence portion
gagagaguagcgcgagcaca

SEQ ID NO: 37 - RNA, 20 nt, Artificial Sequence
  B2M-targeting guide sequence portion
gagaguagcgcgagcacagc

SEQ ID NO: 38 - RNA, 20 nt, Artificial Sequence
  B2M-targeting guide sequence portion
gagaugucucgcuccguggc

SEQ ID NO: 39 - RNA, 20 nt, Artificial Sequence
  B2M-targeting guide sequence portion
gagcacagcuaaggccacgg

SEQ ID NO: 40 - RNA, 20 nt, Artificial Sequence
  B2M-targeting guide sequence portion
gcacagcuaaggccacggag

SEQ ID NO: 41 - RNA, 20 nt, Artificial Sequence
  B2M-targeting guide sequence portion
gccagaaagagagaguagcg

SEQ ID NO: 42 - RNA, 20 nt, Artificial Sequence
  B2M-targeting guide sequence portion
gccgagaugucucgcuccgu

SEQ ID NO: 43 - RNA, 20 nt, Artificial Sequence
  B2M-targeting guide sequence portion
gccuuagcugugcucgcgcu

SEQ ID NO: 44 - RNA, 20 nt, Artificial Sequence
  B2M-targeting guide sequence portion
gcgagcacagcuaaggccac

SEQ ID NO: 45 - RNA, 20 nt, Artificial Sequence
  B2M-targeting guide sequence portion
gcgcgagcacagcuaaggcc

SEQ ID NO: 46 - RNA, 20 nt, Artificial Sequence
  B2M-targeting guide sequence portion
gcuaaggccacggagcgaga

SEQ ID NO: 47 - RNA, 20 nt, Artificial Sequence
  B2M-targeting guide sequence portion
gcuggauagccuccaggcca

SEQ ID NO: 48 - RNA, 20 nt, Artificial Sequence
  B2M-targeting guide sequence portion
ggauagccuccaggccagaa

SEQ ID NO: 49 - RNA, 20 nt, Artificial Sequence
  B2M-targeting guide sequence portion
ggccuggaggcuauccagcg

SEQ ID NO: 50 - RNA, 20 nt, Artificial Sequence
  B2M-targeting guide sequence portion
ggccuuagcugugcucgcgc

SEQ ID NO: 51 - RNA, 20 nt, Artificial Sequence
  B2M-targeting guide sequence portion
guagcgcgagcacagcuaag

SEQ ID NO: 52 - RNA, 20 nt, Artificial Sequence
  B2M-targeting guide sequence portion
gucucgcuccguggccuuag

SEQ ID NO: 53 - RNA, 20 nt, Artificial Sequence
  B2M-targeting guide sequence portion
guggccuuagcugugcucgc

SEQ ID NO: 54 - RNA, 20 nt, Artificial Sequence
  B2M-targeting guide sequence portion
uaaggccacggagcgagaca

SEQ ID NO: 55 - RNA, 20 nt, Artificial Sequence
  B2M-targeting guide sequence portion
uagcgcgagcacagcuaagg

SEQ ID NO: 56 - RNA, 20 nt, Artificial Sequence
  B2M-targeting guide sequence portion
uagcugugcucgcgcuacuc

SEQ ID NO: 57 - RNA, 20 nt, Artificial Sequence
  B2M-targeting guide sequence portion
ucacgcuggauagccuccag

SEQ ID NO: 58 - RNA, 20 nt, Artificial Sequence
  B2M-targeting guide sequence portion
uccguggccuuagcugugcu

SEQ ID NO: 59 - RNA, 20 nt, Artificial Sequence
  B2M-targeting guide sequence portion
ucgcgcuacucucucuuucu

SEQ ID NO: 60 - RNA, 20 nt, Artificial Sequence
  B2M-targeting guide sequence portion
ucgcuccguggccuuagcug

SEQ ID NO: 61 - RNA, 20 nt, Artificial Sequence
  B2M-targeting guide sequence portion
ucucgcuccguggccuuagc

SEQ ID NO: 62 - RNA, 20 nt, Artificial Sequence
  B2M-targeting guide sequence portion
uggccuuagcugugcucgcg

SEQ ID NO: 63 - RNA, 20 nt, Artificial Sequence
  B2M-targeting guide sequence portion
ugucucgcuccguggccuua

SEQ ID NO: 64 - RNA, 20 nt, Artificial Sequence
  B2M-targeting guide sequence portion
uucuggccuggaggcuaucc

SEQ ID NO: 65 - RNA, 21 nt, Artificial Sequence
  B2M-targeting guide sequence portion
aaagagagaguagcgcgagca

SEQ ID NO: 66 - RNA, 21 nt, Artificial Sequence
  B2M-targeting guide sequence portion
aagagagaguagcgcgagcac

SEQ ID NO: 67 - RNA, 21 nt, Artificial Sequence
  B2M-targeting guide sequence portion
aaggccacggagcgagacauc

SEQ ID NO: 68 - RNA, 21 nt, Artificial Sequence
  B2M-targeting guide sequence portion
acagcuaaggccacggagcga

SEQ ID NO: 69 - RNA, 21 nt, Artificial Sequence
  B2M-targeting guide sequence portion
acgcuggauagccuccaggcc

SEQ ID NO: 70 - RNA, 21 nt, Artificial Sequence
  B2M-targeting guide sequence portion
acucacgcuggauagccucca

SEQ ID NO: 71 - RNA, 21 nt, Artificial Sequence
  B2M-targeting guide sequence portion
acucucucuuucuggccugga

SEQ ID NO: 72 - RNA, 21 nt, Artificial Sequence
  B2M-targeting guide sequence portion
agaaagagagaguagcgcgag

SEQ ID NO: 73 - RNA, 21 nt, Artificial Sequence
  B2M-targeting guide sequence portion
agagagaguagcgcgagcaca

SEQ ID NO: 74 - RNA, 21 nt, Artificial Sequence
  B2M-targeting guide sequence portion
agagaguagcgcgagcacagc

SEQ ID NO: 75 - RNA, 21 nt, Artificial Sequence
  B2M-targeting guide sequence portion
agaguagcgcgagcacagcua

SEQ ID NO: 76 - RNA, 21 nt, Artificial Sequence
  B2M-targeting guide sequence portion
agcacagcuaaggccacggag

SEQ ID NO: 77 - RNA, 21 nt, Artificial Sequence
  B2M-targeting guide sequence portion
agccuccaggccagaaagaga

SEQ ID NO: 78 - RNA, 21 nt, Artificial Sequence
  B2M-targeting guide sequence portion
agcgcgagcacagcuaaggcc

SEQ ID NO: 79 - RNA, 21 nt, Artificial Sequence
  B2M-targeting guide sequence portion
agcuaaggccacggagcgaga

SEQ ID NO: 80 - RNA, 21 nt, Artificial Sequence
  B2M-targeting guide sequence portion
aggccacggagcgagacaucu

SEQ ID NO: 81 - RNA, 21 nt, Artificial Sequence
  B2M-targeting guide sequence portion
aggccagaaagagagaguagc

SEQ ID NO: 82 - RNA, 21 nt, Artificial Sequence
  B2M-targeting guide sequence portion
aguagcgcgagcacagcuaag

SEQ ID NO: 83 - RNA, 21 nt, Artificial Sequence
  B2M-targeting guide sequence portion
augucucgcuccguggccuua

SEQ ID NO: 84 - RNA, 21 nt, Artificial Sequence
  B2M-targeting guide sequence portion
cacagcuaaggccacggagcg

SEQ ID NO: 85 - RNA, 21 nt, Artificial Sequence
  B2M-targeting guide sequence portion
cacgcuggauagccuccaggc

SEQ ID NO: 86 - RNA, 21 nt, Artificial Sequence
  B2M-targeting guide sequence portion
cagaaagagagaguagcgcga

SEQ ID NO: 87 - RNA, 21 nt, Artificial Sequence
  B2M-targeting guide sequence portion
cagcuaaggccacggagcgag

SEQ ID NO: 88 - RNA, 21 nt, Artificial Sequence
  B2M-targeting guide sequence portion
caggccagaaagagagaguag

SEQ ID NO: 89 - RNA, 21 nt, Artificial Sequence
  B2M-targeting guide sequence portion
ccagaaagagagaguagcgcg

SEQ ID NO: 90 - RNA, 21 nt, Artificial Sequence
  B2M-targeting guide sequence portion
ccaggccagaaagagagagua

SEQ ID NO: 91 - RNA, 21 nt, Artificial Sequence
  B2M-targeting guide sequence portion
ccgagaugucucgcuccgugg

SEQ ID NO: 92 - RNA, 21 nt, Artificial Sequence
  B2M-targeting guide sequence portion
ccguggccuuagcugugcucg

SEQ ID NO: 93 - RNA, 21 nt, Artificial Sequence
  B2M-targeting guide sequence portion
ccuccaggccagaaagagaga

SEQ ID NO: 94 - RNA, 21 nt, Artificial Sequence
  B2M-targeting guide sequence portion
ccuuagcugugcucgcgcuac

SEQ ID NO: 95 - RNA, 21 nt, Artificial Sequence
  B2M-targeting guide sequence portion
cgagaugucucgcuccguggc

SEQ ID NO: 96 - RNA, 21 nt, Artificial Sequence
  B2M-targeting guide sequence portion
cgagcacagcuaaggccacgg

SEQ ID NO: 97 - RNA, 21 nt, Artificial Sequence
  B2M-targeting guide sequence portion
cgcgagcacagcuaaggccac

SEQ ID NO: 98 - RNA, 21 nt, Artificial Sequence
  B2M-targeting guide sequence portion
cgcuacucucucuuucuggcc

SEQ ID NO: 99 - RNA, 21 nt, Artificial Sequence
  B2M-targeting guide sequence portion
cgcuccguggccuuagcugug

SEQ ID NO: 100 - RNA, 21 nt, Artificial Sequence
  B2M-targeting guide sequence portion
cgcuggauagccuccaggcca

SEQ ID NO: 101 - RNA, 21 nt, Artificial Sequence
  B2M-targeting guide sequence portion
cguggccuuagcugugcucgc

SEQ ID NO: 102 - RNA, 21 nt, Artificial Sequence
  B2M-targeting guide sequence portion
cuaaggccacggagcgagaca

SEQ ID NO: 103 - RNA, 21 nt, Artificial Sequence
  B2M-targeting guide sequence portion
cucgcuccguggccuuagcug

SEQ ID NO: 104 - RNA, 21 nt, Artificial Sequence
  B2M-targeting guide sequence portion
cucucuuucuggccuggaggc

SEQ ID NO: 105 - RNA, 21 nt, Artificial Sequence
  B2M-targeting guide sequence portion
cucuuucuggccuggaggcua

SEQ ID NO: 106 - RNA, 21 nt, Artificial Sequence
  B2M-targeting guide sequence portion
cuggccuggaggcuauccagc

SEQ ID NO: 107 - RNA, 21 nt, Artificial Sequence
  B2M-targeting guide sequence portion
cuuucuggccuggaggcuauc

SEQ ID NO: 108 - RNA, 21 nt, Artificial Sequence
  B2M-targeting guide sequence portion
gaaagagagaguagcgcgagc

SEQ ID NO: 109 - RNA, 21 nt, Artificial Sequence
  B2M-targeting guide sequence portion
gagagaguagcgcgagcacag

SEQ ID NO: 110 - RNA, 21 nt, Artificial Sequence
  B2M-targeting guide sequence portion
gagaguagcgcgagcacagcu

SEQ ID NO: 111 - RNA, 21 nt, Artificial Sequence
  B2M-targeting guide sequence portion
gagaugucucgcuccguggcc

SEQ ID NO: 112 - RNA, 21 nt, Artificial Sequence
  B2M-targeting guide sequence portion
gagcacagcuaaggccacgga

SEQ ID NO: 113 - RNA, 21 nt, Artificial Sequence
  B2M-targeting guide sequence portion
gaguagcgcgagcacagcuaa

SEQ ID NO: 114 - RNA, 21 nt, Artificial Sequence
  B2M-targeting guide sequence portion
gauagccuccaggccagaaag

SEQ ID NO: 115 - RNA, 21 nt, Artificial Sequence
  B2M-targeting guide sequence portion
gaugucucgcuccguggccuu

SEQ ID NO: 116 - RNA, 21 nt, Artificial Sequence
  B2M-targeting guide sequence portion
gcacagcuaaggccacggagc

SEQ ID NO: 117 - RNA, 21 nt, Artificial Sequence
  B2M-targeting guide sequence portion
gccagaaagagagaguagcgc

SEQ ID NO: 118 - RNA, 21 nt, Artificial Sequence
  B2M-targeting guide sequence portion
gccgagaugucucgcuccgug

SEQ ID NO: 119 - RNA, 21 nt, Artificial Sequence
  B2M-targeting guide sequence portion
gccuccaggccagaaagagag

SEQ ID NO: 120 - RNA, 21 nt, Artificial Sequence
  B2M-targeting guide sequence portion
gccuuagcugugcucgcgcua

SEQ ID NO: 121 - RNA, 21 nt, Artificial Sequence
  B2M-targeting guide sequence portion
gcgagcacagcuaaggccacg

SEQ ID NO: 122 - RNA, 21 nt, Artificial Sequence
  B2M-targeting guide sequence portion
gcgcuacucucucuuucuggc

SEQ ID NO: 123 - RNA, 21 nt, Artificial Sequence
  B2M-targeting guide sequence portion
gcuaaggccacggagcgagac

SEQ ID NO: 124 - RNA, 21 nt, Artificial Sequence
  B2M-targeting guide sequence portion
gcuacucucucuuucuggccu

SEQ ID NO: 125 - RNA, 21 nt, Artificial Sequence
  B2M-targeting guide sequence portion
gcuccguggccuuagcugugc

SEQ ID NO: 126 - RNA, 21 nt, Artificial Sequence
  B2M-targeting guide sequence portion
gcuggauagccuccaggccag

SEQ ID NO: 127 - RNA, 21 nt, Artificial Sequence
  B2M-targeting guide sequence portion
gcugugcucgcgcuacucucu

SEQ ID NO: 128 - RNA, 21 nt, Artificial Sequence
  B2M-targeting guide sequence portion
ggauagccuccaggccagaaa

SEQ ID NO: 129 - RNA, 21 nt, Artificial Sequence
  B2M-targeting guide sequence portion
ggccagaaagagagaguagcg

SEQ ID NO: 130 - RNA, 21 nt, Artificial Sequence
  B2M-targeting guide sequence portion
ggccgagaugucucgcuccgu

SEQ ID NO: 131 - RNA, 21 nt, Artificial Sequence
  B2M-targeting guide sequence portion
ggccuuagcugugcucgcgcu

SEQ ID NO: 132 - RNA, 21 nt, Artificial Sequence
  B2M-targeting guide sequence portion
gggccgagaugucucgcuccg

SEQ ID NO: 133 - RNA, 21 nt, Artificial Sequence
  B2M-targeting guide sequence portion
gucucgcuccguggccuuagc

SEQ ID NO: 134 - RNA, 21 nt, Artificial Sequence
  B2M-targeting guide sequence portion
gugcucgcgcuacucucucuu

SEQ ID NO: 135 - RNA, 21 nt, Artificial Sequence
  B2M-targeting guide sequence portion
uaaggccacggagcgagacau

SEQ ID NO: 136 - RNA, 21 nt, Artificial Sequence
  B2M-targeting guide sequence portion
uacucucucuuucuggccugg

SEQ ID NO: 137 - RNA, 21 nt, Artificial Sequence
  B2M-targeting guide sequence portion
uagccuccaggccagaaagag

SEQ ID NO: 138 - RNA, 21 nt, Artificial Sequence
  B2M-targeting guide sequence portion
uagcgcgagcacagcuaaggc

SEQ ID NO: 139 - RNA, 21 nt, Artificial Sequence
  B2M-targeting guide sequence portion
uagcugugcucgcgcuacucu

SEQ ID NO: 140 - RNA, 21 nt, Artificial Sequence
  B2M-targeting guide sequence portion
ucacgcuggauagccuccagg

SEQ ID NO: 141 - RNA, 21 nt, Artificial Sequence
  B2M-targeting guide sequence portion
uccaggccagaaagagagagu

SEQ ID NO: 142 - RNA, 21 nt, Artificial Sequence
  B2M-targeting guide sequence portion
uccguggccuuagcugugcuc

SEQ ID NO: 143 - RNA, 21 nt, Artificial Sequence
  B2M-targeting guide sequence portion
ucgcgcuacucucucuuucug

SEQ ID NO: 144 - RNA, 21 nt, Artificial Sequence
  B2M-targeting guide sequence portion
ucgcuccguggccuuagcugu

SEQ ID NO: 145 - RNA, 21 nt, Artificial Sequence
  B2M-targeting guide sequence portion
ucucgcuccguggccuuagcu

SEQ ID NO: 146 - RNA, 21 nt, Artificial Sequence
  B2M-targeting guide sequence portion
ucucucuuucuggccuggagg

SEQ ID NO: 147 - RNA, 21 nt, Artificial Sequence
  B2M-targeting guide sequence portion
ucucuuucuggccuggaggcu

SEQ ID NO: 148 - RNA, 21 nt, Artificial Sequence
  B2M-targeting guide sequence portion
ucuggccuggaggcuauccag

SEQ ID NO: 149 - RNA, 21 nt, Artificial Sequence
  B2M-targeting guide sequence portion
ucuuucuggccuggaggcuau

SEQ ID NO: 150 - RNA, 21 nt, Artificial Sequence
  B2M-targeting guide sequence portion
uggauagccuccaggccagaa

SEQ ID NO: 151 - RNA, 21 nt, Artificial Sequence
  B2M-targeting guide sequence portion
uggccuuagcugugcucgcgc

SEQ ID NO: 152 - RNA, 21 nt, Artificial Sequence
  B2M-targeting guide sequence portion
ugucucgcuccguggccuuag

SEQ ID NO: 153 - RNA, 21 nt, Artificial Sequence
  B2M-targeting guide sequence portion
uuagcugugcucgcgcuacuc

SEQ ID NO: 154 - RNA, 21 nt, Artificial Sequence
  B2M-targeting guide sequence portion
uucuggccuggaggcuaucca

SEQ ID NO: 155 - RNA, 21 nt, Artificial Sequence
  B2M-targeting guide sequence portion
uuucuggccuggaggcuaucc

SEQ ID NO: 156 - RNA, 22 nt, Artificial Sequence
  B2M-targeting guide sequence portion
aaagagagaguagcgcgagcac

SEQ ID NO: 157 - RNA, 22 nt, Artificial Sequence
  B2M-targeting guide sequence portion
aagagagaguagcgcgagcaca

SEQ ID NO: 158 - RNA, 22 nt, Artificial Sequence
  B2M-targeting guide sequence portion
aaggccacggagcgagacaucu

SEQ ID NO: 159 - RNA, 22 nt, Artificial Sequence
  B2M-targeting guide sequence portion
acagcuaaggccacggagcgag

SEQ ID NO: 160 - RNA, 22 nt, Artificial Sequence
  B2M-targeting guide sequence portion
acgcuggauagccuccaggcca

SEQ ID NO: 161 - RNA, 22 nt, Artificial Sequence
  B2M-targeting guide sequence portion
acucacgcuggauagccuccag

SEQ ID NO: 162 - RNA, 22 nt, Artificial Sequence
  B2M-targeting guide sequence portion
acucucucuuucuggccuggag

SEQ ID NO: 163 - RNA, 22 nt, Artificial Sequence
  B2M-targeting guide sequence portion
agaaagagagaguagcgcgagc

SEQ ID NO: 164 - RNA, 22 nt, Artificial Sequence
  B2M-targeting guide sequence portion
agagagaguagcgcgagcacag

SEQ ID NO: 165 - RNA, 22 nt, Artificial Sequence
  B2M-targeting guide sequence portion
agagaguagcgcgagcacagcu

SEQ ID NO: 166 - RNA, 22 nt, Artificial Sequence
  B2M-targeting guide sequence portion
agaguagcgcgagcacagcuaa

SEQ ID NO: 167 - RNA, 22 nt, Artificial Sequence
  B2M-targeting guide sequence portion
agaugucucgcuccguggccuu

SEQ ID NO: 168 - RNA, 22 nt, Artificial Sequence
  B2M-targeting guide sequence portion
agcacagcuaaggccacggagc

SEQ ID NO: 169 - RNA, 22 nt, Artificial Sequence
  B2M-targeting guide sequence portion
agccuccaggccagaaagagag

SEQ ID NO: 170 - RNA, 22 nt, Artificial Sequence
  B2M-targeting guide sequence portion
agcgcgagcacagcuaaggcca

SEQ ID NO: 171 - RNA, 22 nt, Artificial Sequence
  B2M-targeting guide sequence portion
agcuaaggccacggagcgagac

SEQ ID NO: 172 - RNA, 22 nt, Artificial Sequence
  B2M-targeting guide sequence portion
agcugugcucgcgcuacucucu

SEQ ID NO: 173 - RNA, 22 nt, Artificial Sequence
  B2M-targeting guide sequence portion
auagccuccaggccagaaagag

SEQ ID NO: 174 - RNA, 22 nt, Artificial Sequence
  B2M-targeting guide sequence portion
cacagcuaaggccacggagcga

SEQ ID NO: 175 - RNA, 22 nt, Artificial Sequence
  B2M-targeting guide sequence portion
cacgcuggauagccuccaggcc

SEQ ID NO: 176 - RNA, 22 nt, Artificial Sequence
  B2M-targeting guide sequence portion
cagaaagagagaguagcgcgag

SEQ ID NO: 177 - RNA, 22 nt, Artificial Sequence
  B2M-targeting guide sequence portion
cagcuaaggccacggagcgaga

SEQ ID NO: 178 - RNA, 22 nt, Artificial Sequence
  B2M-targeting guide sequence portion
caggccagaaagagagaguagc

SEQ ID NO: 179 - RNA, 22 nt, Artificial Sequence
  B2M-targeting guide sequence portion
ccagaaagagagaguagcgcga

SEQ ID NO: 180 - RNA, 22 nt, Artificial Sequence
  B2M-targeting guide sequence portion
ccaggccagaaagagagaguag

SEQ ID NO: 181 - RNA, 22 nt, Artificial Sequence
  B2M-targeting guide sequence portion
ccgagaugucucgcuccguggc

SEQ ID NO: 182 - RNA, 22 nt, Artificial Sequence
  B2M-targeting guide sequence portion
ccuccaggccagaaagagagag

SEQ ID NO: 183 - RNA, 22 nt, Artificial Sequence
  B2M-targeting guide sequence portion
cgagaugucucgcuccguggcc

SEQ ID NO: 184 - RNA, 22 nt, Artificial Sequence
  B2M-targeting guide sequence portion
cgagcacagcuaaggccacgga

SEQ ID NO: 185 - RNA, 22 nt, Artificial Sequence
  B2M-targeting guide sequence portion
cgcgagcacagcuaaggccacg

SEQ ID NO: 186 - RNA, 22 nt, Artificial Sequence
  B2M-targeting guide sequence portion
cgcgcuacucucucuuucuggc

SEQ ID NO: 187 - RNA, 22 nt, Artificial Sequence
  B2M-targeting guide sequence portion
cgcuacucucucuuucuggccu

SEQ ID NO: 188 - RNA, 22 nt, Artificial Sequence
  B2M-targeting guide sequence portion
cgcuggauagccuccaggccag

SEQ ID NO: 189 - RNA, 22 nt, Artificial Sequence
  B2M-targeting guide sequence portion
cguggccuuagcugugcucgcg

SEQ ID NO: 190 - RNA, 22 nt, Artificial Sequence
  B2M-targeting guide sequence portion
cuaaggccacggagcgagacau

SEQ ID NO: 191 - RNA, 22 nt, Artificial Sequence
  B2M-targeting guide sequence portion
cuacucucucuuucuggccugg

SEQ ID NO: 192 - RNA, 22 nt, Artificial Sequence
  B2M-targeting guide sequence portion
cucacgcuggauagccuccagg

SEQ ID NO: 193 - RNA, 22 nt, Artificial Sequence
  B2M-targeting guide sequence portion
cuccaggccagaaagagagagu

SEQ ID NO: 194 - RNA, 22 nt, Artificial Sequence
  B2M-targeting guide sequence portion
cucgcuccguggccuuagcugu

SEQ ID NO: 195 - RNA, 22 nt, Artificial Sequence
  B2M-targeting guide sequence portion
cucucucuuucuggccuggagg

SEQ ID NO: 196 - RNA, 22 nt, Artificial Sequence
  B2M-targeting guide sequence portion
cucucuuucuggccuggaggcu

SEQ ID NO: 197 - RNA, 22 nt, Artificial Sequence
  B2M-targeting guide sequence portion
cucuuucuggccuggaggcuau

SEQ ID NO: 198 - RNA, 22 nt, Artificial Sequence
  B2M-targeting guide sequence portion
cuggauagccuccaggccagaa

SEQ ID NO: 199 - RNA, 22 nt, Artificial Sequence
  B2M-targeting guide sequence portion
cuggccuggaggcuauccagcg

SEQ ID NO: 200 - RNA, 22 nt, Artificial Sequence
  B2M-targeting guide sequence portion
cugugcucgcgcuacucucucu

SEQ ID NO: 201 - RNA, 22 nt, Artificial Sequence
  B2M-targeting guide sequence portion
cuuagcugugcucgcgcuacuc

SEQ ID NO: 202 - RNA, 22 nt, Artificial Sequence
  B2M-targeting guide sequence portion
cuuucuggccuggaggcuaucc

SEQ ID NO: 203 - RNA, 22 nt, Artificial Sequence
  B2M-targeting guide sequence portion
gaaagagagaguagcgcgagca

SEQ ID NO: 204 - RNA, 22 nt, Artificial Sequence
  B2M-targeting guide sequence portion
gacucacgcuggauagccucca

SEQ ID NO: 205 - RNA, 22 nt, Artificial Sequence
  B2M-targeting guide sequence portion
gagagaguagcgcgagcacagc

SEQ ID NO: 206 - RNA, 22 nt, Artificial Sequence
  B2M-targeting guide sequence portion
gagaguagcgcgagcacagcua

SEQ ID NO: 207 - RNA, 22 nt, Artificial Sequence
  B2M-targeting guide sequence portion
gagaugucucgcuccguggccu

SEQ ID NO: 208 - RNA, 22 nt, Artificial Sequence
  B2M-targeting guide sequence portion
gagcacagcuaaggccacggag

SEQ ID NO: 209 - RNA, 22 nt, Artificial Sequence
  B2M-targeting guide sequence portion
gaguagcgcgagcacagcuaag

SEQ ID NO: 210 - RNA, 22 nt, Artificial Sequence
  B2M-targeting guide sequence portion
gauagccuccaggccagaaaga

SEQ ID NO: 211 - RNA, 22 nt, Artificial Sequence
  B2M-targeting guide sequence portion
gaugucucgcuccguggccuua

SEQ ID NO: 212 - RNA, 22 nt, Artificial Sequence
  B2M-targeting guide sequence portion
gcacagcuaaggccacggagcg

SEQ ID NO: 213 - RNA, 22 nt, Artificial Sequence
  B2M-targeting guide sequence portion
gccagaaagagagaguagcgcg

SEQ ID NO: 214 - RNA, 22 nt, Artificial Sequence
  B2M-targeting guide sequence portion
gccuccaggccagaaagagaga

SEQ ID NO: 215 - RNA, 22 nt, Artificial Sequence
  B2M-targeting guide sequence portion
gccuuagcugugcucgcgcuac

SEQ ID NO: 216 - RNA, 22 nt, Artificial Sequence
  B2M-targeting guide sequence portion
gcgagcacagcuaaggccacgg

SEQ ID NO: 217 - RNA, 22 nt, Artificial Sequence
  B2M-targeting guide sequence portion
gcgcgagcacagcuaaggccac

SEQ ID NO: 218 - RNA, 22 nt, Artificial Sequence
  B2M-targeting guide sequence portion
gcgcuacucucucuuucuggcc

SEQ ID NO: 219 - RNA, 22 nt, Artificial Sequence
  B2M-targeting guide sequence portion
gcuaaggccacggagcgagaca

SEQ ID NO: 220 - RNA, 22 nt, Artificial Sequence
  B2M-targeting guide sequence portion
gcuacucucucuuucuggccug

SEQ ID NO: 221 - RNA, 22 nt, Artificial Sequence
  B2M-targeting guide sequence portion
gcuccguggccuuagcugugcu

SEQ ID NO: 222 - RNA, 22 nt, Artificial Sequence
  B2M-targeting guide sequence portion
gcucgcgcuacucucucuuucu

SEQ ID NO: 223 - RNA, 22 nt, Artificial Sequence
  B2M-targeting guide sequence portion
gcuggauagccuccaggccaga

SEQ ID NO: 224 - RNA, 22 nt, Artificial Sequence
  B2M-targeting guide sequence portion
gcugugcucgcgcuacucucuc

SEQ ID NO: 225 - RNA, 22 nt, Artificial Sequence
  B2M-targeting guide sequence portion
ggauagccuccaggccagaaag

SEQ ID NO: 226 - RNA, 22 nt, Artificial Sequence
  B2M-targeting guide sequence portion
ggccagaaagagagaguagcgc

SEQ ID NO: 227 - RNA, 22 nt, Artificial Sequence
  B2M-targeting guide sequence portion
ggccgagaugucucgcuccgug

SEQ ID NO: 228 - RNA, 22 nt, Artificial Sequence
  B2M-targeting guide sequence portion
ggccuuagcugugcucgcgcua

SEQ ID NO: 229 - RNA, 22 nt, Artificial Sequence
  B2M-targeting guide sequence portion
gggccgagaugucucgcuccgu

SEQ ID NO: 230 - RNA, 22 nt, Artificial Sequence
  B2M-targeting guide sequence portion
guagcgcgagcacagcuaaggc

SEQ ID NO: 231 - RNA, 22 nt, Artificial Sequence
  B2M-targeting guide sequence portion
gucucgcuccguggccuuagcu

SEQ ID NO: 232 - RNA, 22 nt, Artificial Sequence
  B2M-targeting guide sequence portion
gugcucgcgcuacucucucuuu

SEQ ID NO: 233 - RNA, 22 nt, Artificial Sequence
  B2M-targeting guide sequence portion
guggccuuagcugugcucgcgc

SEQ ID NO: 234 - RNA, 22 nt, Artificial Sequence
  B2M-targeting guide sequence portion
uaaggccacggagcgagacauc

SEQ ID NO: 235 - RNA, 22 nt, Artificial Sequence
  B2M-targeting guide sequence portion
uacucucucuuucuggccugga

SEQ ID NO: 236 - RNA, 22 nt, Artificial Sequence
  B2M-targeting guide sequence portion
uagccuccaggccagaaagaga

SEQ ID NO: 237 - RNA, 22 nt, Artificial Sequence
  B2M-targeting guide sequence portion
uagcgcgagcacagcuaaggcc

SEQ ID NO: 238 - RNA, 22 nt, Artificial Sequence
  B2M-targeting guide sequence portion
uagcugugcucgcgcuacucuc

SEQ ID NO: 239 - RNA, 22 nt, Artificial Sequence
  B2M-targeting guide sequence portion
ucacgcuggauagccuccaggc

SEQ ID NO: 240 - RNA, 22 nt, Artificial Sequence
  B2M-targeting guide sequence portion
uccaggccagaaagagagagua

SEQ ID NO: 241 - RNA, 22 nt, Artificial Sequence
  B2M-targeting guide sequence portion
uccguggccuuagcugugcucg

SEQ ID NO: 242 - RNA, 22 nt, Artificial Sequence
  B2M-targeting guide sequence portion
ucgcgcuacucucucuuucugg

SEQ ID NO: 243 - RNA, 22 nt, Artificial Sequence
  B2M-targeting guide sequence portion
ucgcuccguggccuuagcugug

SEQ ID NO: 244 - RNA, 22 nt, Artificial Sequence
  B2M-targeting guide sequence portion
ucucucuuucuggccuggaggc

SEQ ID NO: 245 - RNA, 22 nt, Artificial Sequence
  B2M-targeting guide sequence portion
ucucuuucuggccuggaggcua

SEQ ID NO: 246 - RNA, 22 nt, Artificial Sequence
  B2M-targeting guide sequence portion
ucuggccuggaggcuauccagc

SEQ ID NO: 247 - RNA, 22 nt, Artificial Sequence
  B2M-targeting guide sequence portion
uggauagccuccaggccagaaa

SEQ ID NO: 248 - RNA, 22 nt, Artificial Sequence
  B2M-targeting guide sequence portion
uggccuuagcugugcucgcgcu

SEQ ID NO: 249 - RNA, 22 nt, Artificial Sequence
  B2M-targeting guide sequence portion
ugucucgcuccguggccuuagc

SEQ ID NO: 250 - RNA, 22 nt, Artificial Sequence
  B2M-targeting guide sequence portion
ugugcucgcgcuacucucucuu

SEQ ID NO: 251 - RNA, 22 nt, Artificial Sequence
  B2M-targeting guide sequence portion
uuagcugugcucgcgcuacucu

SEQ ID NO: 252 - RNA, 22 nt, Artificial Sequence
  B2M-targeting guide sequence portion
uucuggccuggaggcuauccag

SEQ ID NO: 253 - RNA, 22 nt, Artificial Sequence
  B2M-targeting guide sequence portion
uuucuggccuggaggcuaucca

SEQ ID NO: 254 - RNA, 20 nt, Artificial Sequence
  B2M-targeting guide sequence portion
aaaaguggagcauucagacu

SEQ ID NO: 255 - RNA, 20 nt, Artificial Sequence
  B2M-targeting guide sequence portion
aaacccagacacauagcaau

SEQ ID NO: 256 - RNA, 20 nt, Artificial Sequence
  B2M-targeting guide sequence portion
aaaccugaaucuuuggagua

SEQ ID NO: 257 - RNA, 20 nt, Artificial Sequence
  B2M-targeting guide sequence portion
aaagaccaguccuugcugaa

SEQ ID NO: 258 - RNA, 20 nt, Artificial Sequence
  B2M-targeting guide sequence portion
aaagauucagguuuacucac

SEQ ID NO: 259 - RNA, 20 nt, Artificial Sequence
  B2M-targeting guide sequence portion
aaagucaaauuuccugaauu

SEQ ID NO: 260 - RNA, 20 nt, Artificial Sequence
  B2M-targeting guide sequence portion
aaagucacaugguucacacg

SEQ ID NO: 261 - RNA, 20 nt, Artificial Sequence
  B2M-targeting guide sequence portion
aaaguggagcauucagacuu

SEQ ID NO: 262 - RNA, 20 nt, Artificial Sequence
  B2M-targeting guide sequence portion
aaauuuccugaauugcuaug

SEQ ID NO: 263 - RNA, 20 nt, Artificial Sequence
  B2M-targeting guide sequence portion
aaauuugacuuuccauucuc

SEQ ID NO: 264 - RNA, 20 nt, Artificial Sequence
  B2M-targeting guide sequence portion
aaccaugugacuuugucaca

SEQ ID NO: 265 - RNA, 20 nt, Artificial Sequence
  B2M-targeting guide sequence portion
aaccugaaucuuuggaguac

SEQ ID NO: 266 - RNA, 20 nt, Artificial Sequence
  B2M-targeting guide sequence portion
aagacaagucugaaugcucc

SEQ ID NO: 267 - RNA, 20 nt, Artificial Sequence
  B2M-targeting guide sequence portion
aagaccaguccuugcugaaa

SEQ ID NO: 268 - RNA, 20 nt, Artificial Sequence
  B2M-targeting guide sequence portion
aagagauagaaagaccaguc

SEQ ID NO: 269 - RNA, 20 nt, Artificial Sequence
  B2M-targeting guide sequence portion
aagauucagguuuacucacg

SEQ ID NO: 270 - RNA, 20 nt, Artificial Sequence
  B2M-targeting guide sequence portion
aaggacuggucuuucuaucu

SEQ ID NO: 271 - RNA, 20 nt, Artificial Sequence
  B2M-targeting guide sequence portion
aagucaaauuuccugaauug

SEQ ID NO: 272 - RNA, 20 nt, Artificial Sequence
  B2M-targeting guide sequence portion
aagucugaaugcuccacuuu

SEQ ID NO: 273 - RNA, 20 nt, Artificial Sequence
  B2M-targeting guide sequence portion
aaguggagcauucagacuug

SEQ ID NO: 274 - RNA, 20 nt, Artificial Sequence
  B2M-targeting guide sequence portion
aauggaaagucaaauuuccu

SEQ ID NO: 275 - RNA, 20 nt, Artificial Sequence
  B2M-targeting guide sequence portion
aauggagagagaauugaaaa

SEQ ID NO: 276 - RNA, 20 nt, Artificial Sequence
  B2M-targeting guide sequence portion
aauucaggaaauuugacuuu

SEQ ID NO: 277 - RNA, 20 nt, Artificial Sequence
  B2M-targeting guide sequence portion
aauuuccugaauugcuaugu

SEQ ID NO: 278 - RNA, 20 nt, Artificial Sequence
  B2M-targeting guide sequence portion
aauuugacuuuccauucucu

SEQ ID NO: 279 - RNA, 20 nt, Artificial Sequence
  B2M-targeting guide sequence portion
acaagagauagaaagaccag

SEQ ID NO: 280 - RNA, 20 nt, Artificial Sequence
  B2M-targeting guide sequence portion
acacauagcaauucaggaaa

SEQ ID NO: 281 - RNA, 20 nt, Artificial Sequence
  B2M-targeting guide sequence portion
acauagcaauucaggaaauu

SEQ ID NO: 282 - RNA, 20 nt, Artificial Sequence
  B2M-targeting guide sequence portion
acaugguucacacggcaggc

SEQ ID NO: 283 - RNA, 20 nt, Artificial Sequence
  B2M-targeting guide sequence portion
accaugugacuuugucacag

SEQ ID NO: 284 - RNA, 20 nt, Artificial Sequence
  B2M-targeting guide sequence portion
accugaaucuuuggaguacc

SEQ ID NO: 285 - RNA, 20 nt, Artificial Sequence
  B2M-targeting guide sequence portion
acgugaguaaaccugaaucu

SEQ ID NO: 286 - RNA, 20 nt, Artificial Sequence
  B2M-targeting guide sequence portion
acuacacugaauucaccccc

SEQ ID NO: 287 - RNA, 20 nt, Artificial Sequence
  B2M-targeting guide sequence portion
acuccaaagauucagguuua

SEQ ID NO: 288 - RNA, 20 nt, Artificial Sequence
  B2M-targeting guide sequence portion
acugaaaaagaugaguaugc

SEQ ID NO: 289 - RNA, 20 nt, Artificial Sequence
  B2M-targeting guide sequence portion
acuggucuuucuaucucuug

SEQ ID NO: 290 - RNA, 20 nt, Artificial Sequence
  B2M-targeting guide sequence portion
acuuaacuaucuugggcugu

SEQ ID NO: 291 - RNA, 20 nt, Artificial Sequence
  B2M-targeting guide sequence portion
acuuaccccacuuaacuauc

SEQ ID NO: 292 - RNA, 20 nt, Artificial Sequence
  B2M-targeting guide sequence portion
acuucaaugucggauggaug

SEQ ID NO: 293 - RNA, 20 nt, Artificial Sequence
  B2M-targeting guide sequence portion
acuuugucacagcccaagau

SEQ ID NO: 294 - RNA, 20 nt, Artificial Sequence
  B2M-targeting guide sequence portion
acuuuuucaauucucucucc

SEQ ID NO: 295 - RNA, 20 nt, Artificial Sequence
  B2M-targeting guide sequence portion
agaaagaccaguccuugcug

SEQ ID NO: 296 - RNA, 20 nt, Artificial Sequence
  B2M-targeting guide sequence portion
agaauggagagagaauugaa

SEQ ID NO: 297 - RNA, 20 nt, Artificial Sequence
  B2M-targeting guide sequence portion
agaauugaaaaaguggagca

SEQ ID NO: 298 - RNA, 20 nt, Artificial Sequence
  B2M-targeting guide sequence portion
agacaagucugaaugcucca

SEQ ID NO: 299 - RNA, 20 nt, Artificial Sequence
  B2M-targeting guide sequence portion
agacacauagcaauucagga

SEQ ID NO: 300 - RNA, 20 nt, Artificial Sequence
  B2M-targeting guide sequence portion
agaccaguccuugcugaaag

SEQ ID NO: 301 - RNA, 20 nt, Artificial Sequence
  B2M-targeting guide sequence portion
agagaauggaaagucaaauu

SEQ ID NO: 302 - RNA, 20 nt, Artificial Sequence
  B2M-targeting guide sequence portion
agagaauugaaaaaguggag

SEQ ID NO: 303 - RNA, 20 nt, Artificial Sequence
  B2M-targeting guide sequence portion
agagagaauugaaaaagugg

SEQ ID NO: 304 - RNA, 20 nt, Artificial Sequence
  B2M-targeting guide sequence portion
agagauagaaagaccagucc

SEQ ID NO: 305 - RNA, 20 nt, Artificial Sequence
  B2M-targeting guide sequence portion
agauagaaagaccaguccuu

SEQ ID NO: 306 - RNA, 20 nt, Artificial Sequence
  B2M-targeting guide sequence portion
agauucagguuuacucacgu

SEQ ID NO: 307 - RNA, 20 nt, Artificial Sequence
  B2M-targeting guide sequence portion
agcaauucaggaaauuugac

SEQ ID NO: 308 - RNA, 20 nt, Artificial Sequence
  B2M-targeting guide sequence portion
agcagagaauggaaagucaa

SEQ ID NO: 309 - RNA, 20 nt, Artificial Sequence
  B2M-targeting guide sequence portion
agcauucagacuugucuuuc

SEQ ID NO: 310 - RNA, 20 nt, Artificial Sequence
  B2M-targeting guide sequence portion
agcccaagauaguuaagugg

SEQ ID NO: 311 - RNA, 20 nt, Artificial Sequence
  B2M-targeting guide sequence portion
aggacuggucuuucuaucuc

SEQ ID NO: 312 - RNA, 20 nt, Artificial Sequence
  B2M-targeting guide sequence portion
aggcauacucaucuuuuuca

SEQ ID NO: 313 - RNA, 20 nt, Artificial Sequence
  B2M-targeting guide sequence portion
aguaaaccugaaucuuugga

SEQ ID NO: 314 - RNA, 20 nt, Artificial Sequence
  B2M-targeting guide sequence portion
aguaugccugccgugugaac

SEQ ID NO: 315 - RNA, 20 nt, Artificial Sequence
  B2M-targeting guide sequence portion
agucaaauuuccugaauugc

SEQ ID NO: 316 - RNA, 20 nt, Artificial Sequence
  B2M-targeting guide sequence portion
agucaacuucaaugucggau

SEQ ID NO: 317 - RNA, 20 nt, Artificial Sequence
  B2M-targeting guide sequence portion
aguccuugcugaaagacaag

SEQ ID NO: 318 - RNA, 20 nt, Artificial Sequence
  B2M-targeting guide sequence portion
agucugaaugcuccacuuuu

SEQ ID NO: 319 - RNA, 20 nt, Artificial Sequence
  B2M-targeting guide sequence portion
aguggagcauucagacuugu

SEQ ID NO: 320 - RNA, 20 nt, Artificial Sequence
  B2M-targeting guide sequence portion
auagaaagaccaguccuugc

SEQ ID NO: 321 - RNA, 20 nt, Artificial Sequence
  B2M-targeting guide sequence portion
auagcaauucaggaaauuug

SEQ ID NO: 322 - RNA, 20 nt, Artificial Sequence
  B2M-targeting guide sequence portion
auccagcagagaauggaaag

SEQ ID NO: 323 - RNA, 20 nt, Artificial Sequence
  B2M-targeting guide sequence portion
auccgacauugaaguugacu

SEQ ID NO: 324 - RNA, 20 nt, Artificial Sequence
  B2M-targeting guide sequence portion
aucucuuguacuacacugaa

SEQ ID NO: 325 - RNA, 20 nt, Artificial Sequence
  B2M-targeting guide sequence portion
aucuugggcugugacaaagu

SEQ ID NO: 326 - RNA, 20 nt, Artificial Sequence
  B2M-targeting guide sequence portion
augaaacccagacacauagc

SEQ ID NO: 327 - RNA, 20 nt, Artificial Sequence
  B2M-targeting guide sequence portion
augacgugaguaaaccugaa

SEQ ID NO: 328 - RNA, 20 nt, Artificial Sequence
  B2M-targeting guide sequence portion
augccugccgugugaaccau

SEQ ID NO: 329 - RNA, 20 nt, Artificial Sequence
  B2M-targeting guide sequence portion
augcuccacuuuuucaauuc

SEQ ID NO: 330 - RNA, 20 nt, Artificial Sequence
  B2M-targeting guide sequence portion
auggaaagucaaauuuccug

SEQ ID NO: 331 - RNA, 20 nt, Artificial Sequence
  B2M-targeting guide sequence portion
auggagagagaauugaaaaa

SEQ ID NO: 332 - RNA, 20 nt, Artificial Sequence
  B2M-targeting guide sequence portion
augguucacacggcaggcau

SEQ ID NO: 333 - RNA, 20 nt, Artificial Sequence
  B2M-targeting guide sequence portion
augucggauggaugaaaccc

SEQ ID NO: 334 - RNA, 20 nt, Artificial Sequence
  B2M-targeting guide sequence portion
augugucuggguuucaucca

SEQ ID NO: 335 - RNA, 20 nt, Artificial Sequence
  B2M-targeting guide sequence portion
auucaggaaauuugacuuuc

SEQ ID NO: 336 - RNA, 20 nt, Artificial Sequence
  B2M-targeting guide sequence portion
auucagguuuacucacguca

SEQ ID NO: 337 - RNA, 20 nt, Artificial Sequence
  B2M-targeting guide sequence portion
auucucucuccauucuucag

SEQ ID NO: 338 - RNA, 20 nt, Artificial Sequence
  B2M-targeting guide sequence portion
auucucugcuggaugacgug

SEQ ID NO: 339 - RNA, 20 nt, Artificial Sequence
  B2M-targeting guide sequence portion
auucuucaguaagucaacuu

SEQ ID NO: 340 - RNA, 20 nt, Artificial Sequence
  B2M-targeting guide sequence portion
auugaaaaaguggagcauuc

SEQ ID NO: 341 - RNA, 20 nt, Artificial Sequence
  B2M-targeting guide sequence portion
auugaaguugacuuacugaa

SEQ ID NO: 342 - RNA, 20 nt, Artificial Sequence
  B2M-targeting guide sequence portion
auugcuaugugucuggguuu

SEQ ID NO: 343 - RNA, 20 nt, Artificial Sequence
  B2M-targeting guide sequence portion
auuuccugaauugcuaugug

SEQ ID NO: 344 - RNA, 20 nt, Artificial Sequence
  B2M-targeting guide sequence portion
caaagucacaugguucacac

SEQ ID NO: 345 - RNA, 20 nt, Artificial Sequence
  B2M-targeting guide sequence portion
caaauuuccugaauugcuau

SEQ ID NO: 346 - RNA, 20 nt, Artificial Sequence
  B2M-targeting guide sequence portion
caacuucaaugucggaugga

SEQ ID NO: 347 - RNA, 20 nt, Artificial Sequence
  B2M-targeting guide sequence portion
caagagauagaaagaccagu

SEQ ID NO: 348 - RNA, 20 nt, Artificial Sequence
  B2M-targeting guide sequence portion
caaggacuggucuuucuauc

SEQ ID NO: 349 - RNA, 20 nt, Artificial Sequence
  B2M-targeting guide sequence portion
caagucugaaugcuccacuu

SEQ ID NO: 350 - RNA, 20 nt, Artificial Sequence
  B2M-targeting guide sequence portion
caauucaggaaauuugacuu

SEQ ID NO: 351 - RNA, 20 nt, Artificial Sequence
  B2M-targeting guide sequence portion
cacacggcaggcauacucau

SEQ ID NO: 352 - RNA, 20 nt, Artificial Sequence
  B2M-targeting guide sequence portion
cacauagcaauucaggaaau

SEQ ID NO: 353 - RNA, 20 nt, Artificial Sequence
  B2M-targeting guide sequence portion
cacaugguucacacggcagg

SEQ ID NO: 354 - RNA, 20 nt, Artificial Sequence
  B2M-targeting guide sequence portion
cacccccacugaaaaagaug

SEQ ID NO: 355 - RNA, 20 nt, Artificial Sequence
  B2M-targeting guide sequence portion
cacggcaggcauacucaucu

SEQ ID NO: 356 - RNA, 20 nt, Artificial Sequence
  B2M-targeting guide sequence portion
cacgucauccagcagagaau

SEQ ID NO: 357 - RNA, 20 nt, Artificial Sequence
  B2M-targeting guide sequence portion
cacugaaaaagaugaguaug

SEQ ID NO: 358 - RNA, 20 nt, Artificial Sequence
  B2M-targeting guide sequence portion
cacuuaacuaucuugggcug

SEQ ID NO: 359 - RNA, 20 nt, Artificial Sequence
  B2M-targeting guide sequence portion
cagacacauagcaauucagg

SEQ ID NO: 360 - RNA, 20 nt, Artificial Sequence
  B2M-targeting guide sequence portion
cagacuugucuuucagcaag

SEQ ID NO: 361 - RNA, 20 nt, Artificial Sequence
  B2M-targeting guide sequence portion
cagagaauggaaagucaaau

SEQ ID NO: 362 - RNA, 20 nt, Artificial Sequence
  B2M-targeting guide sequence portion
cagcagagaauggaaaguca

SEQ ID NO: 363 - RNA, 20 nt, Artificial Sequence
  B2M-targeting guide sequence portion
caggaaauuugacuuuccau

SEQ ID NO: 364 - RNA, 20 nt, Artificial Sequence
  B2M-targeting guide sequence portion
caggcauacucaucuuuuuc

SEQ ID NO: 365 - RNA, 20 nt, Artificial Sequence
  B2M-targeting guide sequence portion
cagguacuccaaagauucag

SEQ ID NO: 366 - RNA, 20 nt, Artificial Sequence
  B2M-targeting guide sequence portion
cagguuuacucacgucaucc

SEQ ID NO: 367 - RNA, 20 nt, Artificial Sequence
  B2M-targeting guide sequence portion
caguccuugcugaaagacaa

SEQ ID NO: 368 - RNA, 20 nt, Artificial Sequence
  B2M-targeting guide sequence portion
caguguaguacaagagauag

SEQ ID NO: 369 - RNA, 20 nt, Artificial Sequence
  B2M-targeting guide sequence portion
cauagcaauucaggaaauuu

SEQ ID NO: 370 - RNA, 20 nt, Artificial Sequence
  B2M-targeting guide sequence portion
cauccagcagagaauggaaa

SEQ ID NO: 371 - RNA, 20 nt, Artificial Sequence
  B2M-targeting guide sequence portion
cauccgacauugaaguugac

SEQ ID NO: 372 - RNA, 20 nt, Artificial Sequence
  B2M-targeting guide sequence portion
caucuuuuucagugggggug

SEQ ID NO: 373 - RNA, 20 nt, Artificial Sequence
  B2M-targeting guide sequence portion
caugguucacacggcaggca

SEQ ID NO: 374 - RNA, 20 nt, Artificial Sequence
  B2M-targeting guide sequence portion
caugugacuuugucacagcc

SEQ ID NO: 375 - RNA, 20 nt, Artificial Sequence
  B2M-targeting guide sequence portion
cauucagacuugucuuucag

SEQ ID NO: 376 - RNA, 20 nt, Artificial Sequence
  B2M-targeting guide sequence portion
cauugaaguugacuuacuga

SEQ ID NO: 377 - RNA, 20 nt, Artificial Sequence
  B2M-targeting guide sequence portion
ccaaagauucagguuuacuc

SEQ ID NO: 378 - RNA, 20 nt, Artificial Sequence
  B2M-targeting guide sequence portion
ccacugaaaaagaugaguau

SEQ ID NO: 379 - RNA, 20 nt, Artificial Sequence
  B2M-targeting guide sequence portion
ccacuuaacuaucuugggcu

SEQ ID NO: 380 - RNA, 20 nt, Artificial Sequence
  B2M-targeting guide sequence portion
ccacuuuuucaauucucucu

SEQ ID NO: 381 - RNA, 20 nt, Artificial Sequence
  B2M-targeting guide sequence portion
ccagacacauagcaauucag

SEQ ID NO: 382 - RNA, 20 nt, Artificial Sequence
  B2M-targeting guide sequence portion
ccaugugacuuugucacagc

SEQ ID NO: 383 - RNA, 20 nt, Artificial Sequence
  B2M-targeting guide sequence portion
ccauucuucaguaagucaac

SEQ ID NO: 384 - RNA, 20 nt, Artificial Sequence
  B2M-targeting guide sequence portion
cccaagauaguuaagugggg

SEQ ID NO: 385 - RNA, 20 nt, Artificial Sequence
  B2M-targeting guide sequence portion
cccacugaaaaagaugagua

SEQ ID NO: 386 - RNA, 20 nt, Artificial Sequence
  B2M-targeting guide sequence portion
cccacuuaacuaucuugggc

SEQ ID NO: 387 - RNA, 20 nt, Artificial Sequence
  B2M-targeting guide sequence portion
cccagacacauagcaauuca

SEQ ID NO: 388 - RNA, 20 nt, Artificial Sequence
  B2M-targeting guide sequence portion
ccccacugaaaaagaugagu

SEQ ID NO: 389 - RNA, 20 nt, Artificial Sequence
  B2M-targeting guide sequence portion
ccgacauugaaguugacuua

SEQ ID NO: 390 - RNA, 20 nt, Artificial Sequence
  B2M-targeting guide sequence portion
ccugccgugugaaccaugug

SEQ ID NO: 391 - RNA, 20 nt, Artificial Sequence
  B2M-targeting guide sequence portion
ccuugcugaaagacaagucu

SEQ ID NO: 392 - RNA, 20 nt, Artificial Sequence
  B2M-targeting guide sequence portion
cgacauugaaguugacuuac

SEQ ID NO: 393 - RNA, 20 nt, Artificial Sequence
  B2M-targeting guide sequence portion
cggauggaugaaacccagac

SEQ ID NO: 394 - RNA, 20 nt, Artificial Sequence
  B2M-targeting guide sequence portion
cgucauccagcagagaaugg

SEQ ID NO: 395 - RNA, 20 nt, Artificial Sequence
  B2M-targeting guide sequence portion
cgugugaaccaugugacuuu

SEQ ID NO: 396 - RNA, 20 nt, Artificial Sequence
  B2M-targeting guide sequence portion
cuacacugaauucaccccca

SEQ ID NO: 397 - RNA, 20 nt, Artificial Sequence
  B2M-targeting guide sequence portion
cuaucuugggcugugacaaa

SEQ ID NO: 398 - RNA, 20 nt, Artificial Sequence
  B2M-targeting guide sequence portion
cucaucuuuuucaguggggg

SEQ ID NO: 399 - RNA, 20 nt, Artificial Sequence
  B2M-targeting guide sequence portion
cuccaaagauucagguuuac

SEQ ID NO: 400 - RNA, 20 nt, Artificial Sequence
  B2M-targeting guide sequence portion
cuccacuuuuucaauucucu

SEQ ID NO: 401 - RNA, 20 nt, Artificial Sequence
  B2M-targeting guide sequence portion
cuccauucuucaguaaguca

SEQ ID NO: 402 - RNA, 20 nt, Artificial Sequence
  B2M-targeting guide sequence portion
cucuccauucuucaguaagu

SEQ ID NO: 403 - RNA, 20 nt, Artificial Sequence
  B2M-targeting guide sequence portion
cucucuccauucuucaguaa

SEQ ID NO: 404 - RNA, 20 nt, Artificial Sequence
  B2M-targeting guide sequence portion
cucugcuggaugacgugagu

SEQ ID NO: 405 - RNA, 20 nt, Artificial Sequence
  B2M-targeting guide sequence portion
cucuuguacuacacugaauu

SEQ ID NO: 406 - RNA, 20 nt, Artificial Sequence
  B2M-targeting guide sequence portion
cugaaugcuccacuuuuuca

SEQ ID NO: 407 - RNA, 20 nt, Artificial Sequence
  B2M-targeting guide sequence portion
cugaauucacccccacugaa

SEQ ID NO: 408 - RNA, 20 nt, Artificial Sequence
  B2M-targeting guide sequence portion
cugccgugugaaccauguga

SEQ ID NO: 409 - RNA, 20 nt, Artificial Sequence
  B2M-targeting guide sequence portion
cugcuggaugacgugaguaa

SEQ ID NO: 410 - RNA, 20 nt, Artificial Sequence
  B2M-targeting guide sequence portion
cuggguuucauccauccgac

SEQ ID NO: 411 - RNA, 20 nt, Artificial Sequence
  B2M-targeting guide sequence portion
cuggucuuucuaucucuugu

SEQ ID NO: 412 - RNA, 20 nt, Artificial Sequence
  B2M-targeting guide sequence portion
cugugacaaagucacauggu

SEQ ID NO: 413 - RNA, 20 nt, Artificial Sequence
  B2M-targeting guide sequence portion
cuuaacuaucuugggcugug

SEQ ID NO: 414 - RNA, 20 nt, Artificial Sequence
  B2M-targeting guide sequence portion
cuuacugaagaauggagaga

SEQ ID NO: 415 - RNA, 20 nt, Artificial Sequence
  B2M-targeting guide sequence portion
cuucaaugucggauggauga

SEQ ID NO: 416 - RNA, 20 nt, Artificial Sequence
  B2M-targeting guide sequence portion
cuucaguaagucaacuucaa

SEQ ID NO: 417 - RNA, 20 nt, Artificial Sequence
  B2M-targeting guide sequence portion
cuugcugaaagacaagucug

SEQ ID NO: 418 - RNA, 20 nt, Artificial Sequence
  B2M-targeting guide sequence portion
cuugggcugugacaaaguca

SEQ ID NO: 419 - RNA, 20 nt, Artificial Sequence
  B2M-targeting guide sequence portion
cuuguacuacacugaauuca

SEQ ID NO: 420 - RNA, 20 nt, Artificial Sequence
  B2M-targeting guide sequence portion
cuugucuuucagcaaggacu

SEQ ID NO: 421 - RNA, 20 nt, Artificial Sequence
  B2M-targeting guide sequence portion
cuuucagcaaggacuggucu

SEQ ID NO: 422 - RNA, 20 nt, Artificial Sequence
  B2M-targeting guide sequence portion
cuuuccauucucugcuggau

SEQ ID NO: 423 - RNA, 20 nt, Artificial Sequence
  B2M-targeting guide sequence portion
cuuucuaucucuuguacuac

SEQ ID NO: 424 - RNA, 20 nt, Artificial Sequence
  B2M-targeting guide sequence portion
cuuugucacagcccaagaua

SEQ ID NO: 425 - RNA, 20 nt, Artificial Sequence
  B2M-targeting guide sequence portion
cuuuuucaauucucucucca

SEQ ID NO: 426 - RNA, 20 nt, Artificial Sequence
  B2M-targeting guide sequence portion
cuuuuucagugggggugaau

SEQ ID NO: 427 - RNA, 20 nt, Artificial Sequence
  B2M-targeting guide sequence portion
gaaaaagaugaguaugccug

SEQ ID NO: 428 - RNA, 20 nt, Artificial Sequence
  B2M-targeting guide sequence portion
gaaaaaguggagcauucaga

SEQ ID NO: 429 - RNA, 20 nt, Artificial Sequence
  B2M-targeting guide sequence portion
gaaacccagacacauagcaa

SEQ ID NO: 430 - RNA, 20 nt, Artificial Sequence
  B2M-targeting guide sequence portion
gaaagacaagucugaaugcu

SEQ ID NO: 431 - RNA, 20 nt, Artificial Sequence
  B2M-targeting guide sequence portion
gaaagucaaauuuccugaau

SEQ ID NO: 432 - RNA, 20 nt, Artificial Sequence
  B2M-targeting guide sequence portion
gaaauuugacuuuccauucu

SEQ ID NO: 433 - RNA, 20 nt, Artificial Sequence
  B2M-targeting guide sequence portion
gaaccaugugacuuugucac

SEQ ID NO: 434 - RNA, 20 nt, Artificial Sequence
  B2M-targeting guide sequence portion
gaagaauggagagagaauug

SEQ ID NO: 435 - RNA, 20 nt, Artificial Sequence
  B2M-targeting guide sequence portion
gaaugcuccacuuuuucaau

SEQ ID NO: 436 - RNA, 20 nt, Artificial Sequence
  B2M-targeting guide sequence portion
gaauggaaagucaaauuucc

SEQ ID NO: 437 - RNA, 20 nt, Artificial Sequence
  B2M-targeting guide sequence portion
gaauucaguguaguacaaga

SEQ ID NO: 438 - RNA, 20 nt, Artificial Sequence
  B2M-targeting guide sequence portion
gaauugaaaaaguggagcau

SEQ ID NO: 439 - RNA, 20 nt, Artificial Sequence
  B2M-targeting guide sequence portion
gaauugcuaugugucugggu

SEQ ID NO: 440 - RNA, 20 nt, Artificial Sequence
  B2M-targeting guide sequence portion
gacaaagucacaugguucac

SEQ ID NO: 441 - RNA, 20 nt, Artificial Sequence
  B2M-targeting guide sequence portion
gacacauagcaauucaggaa

SEQ ID NO: 442 - RNA, 20 nt, Artificial Sequence
  B2M-targeting guide sequence portion
gaccaguccuugcugaaaga

SEQ ID NO: 443 - RNA, 20 nt, Artificial Sequence
  B2M-targeting guide sequence portion
gacgugaguaaaccugaauc

SEQ ID NO: 444 - RNA, 20 nt, Artificial Sequence
  B2M-targeting guide sequence portion
gacuggucuuucuaucucuu

SEQ ID NO: 445 - RNA, 20 nt, Artificial Sequence
  B2M-targeting guide sequence portion
gacuuaccccacuuaacuau

SEQ ID NO: 446 - RNA, 20 nt, Artificial Sequence
  B2M-targeting guide sequence portion
gagaauggaaagucaaauuu

SEQ ID NO: 447 - RNA, 20 nt, Artificial Sequence
  B2M-targeting guide sequence portion
gagaauugaaaaaguggagc

SEQ ID NO: 448 - RNA, 20 nt, Artificial Sequence
  B2M-targeting guide sequence portion
gagagaauugaaaaagugga

SEQ ID NO: 449 - RNA, 20 nt, Artificial Sequence
  B2M-targeting guide sequence portion
gagauagaaagaccaguccu

SEQ ID NO: 450 - RNA, 20 nt, Artificial Sequence
  B2M-targeting guide sequence portion
gaguaaaccugaaucuuugg

SEQ ID NO: 451 - RNA, 20 nt, Artificial Sequence
  B2M-targeting guide sequence portion
gaguaugccugccgugugaa

SEQ ID NO: 452 - RNA, 20 nt, Artificial Sequence
  B2M-targeting guide sequence portion
gauagaaagaccaguccuug

SEQ ID NO: 453 - RNA, 20 nt, Artificial Sequence
  B2M-targeting guide sequence portion
gaugaaacccagacacauag

SEQ ID NO: 454 - RNA, 20 nt, Artificial Sequence
  B2M-targeting guide sequence portion
gauucagguuuacucacguc

SEQ ID NO: 455 - RNA, 20 nt, Artificial Sequence
  B2M-targeting guide sequence portion
gcaaggacuggucuuucuau

SEQ ID NO: 456 - RNA, 20 nt, Artificial Sequence
  B2M-targeting guide sequence portion
gcaauucaggaaauuugacu

SEQ ID NO: 457 - RNA, 20 nt, Artificial Sequence
  B2M-targeting guide sequence portion
gcagagaauggaaagucaaa

SEQ ID NO: 458 - RNA, 20 nt, Artificial Sequence
  B2M-targeting guide sequence portion
gcauucagacuugucuuuca

SEQ ID NO: 459 - RNA, 20 nt, Artificial Sequence
  B2M-targeting guide sequence portion
gcccaagauaguuaaguggg

SEQ ID NO: 460 - RNA, 20 nt, Artificial Sequence
  B2M-targeting guide sequence portion
gccgugugaaccaugugacu

SEQ ID NO: 461 - RNA, 20 nt, Artificial Sequence
  B2M-targeting guide sequence portion
gcuaugugucuggguuucau

SEQ ID NO: 462 - RNA, 20 nt, Artificial Sequence
  B2M-targeting guide sequence portion
gcuccacuuuuucaauucuc

SEQ ID NO: 463 - RNA, 20 nt, Artificial Sequence
  B2M-targeting guide sequence portion
ggaaagucaaauuuccugaa

SEQ ID NO: 464 - RNA, 20 nt, Artificial Sequence
  B2M-targeting guide sequence portion
ggaaauuugacuuuccauuc

SEQ ID NO: 465 - RNA, 20 nt, Artificial Sequence
  B2M-targeting guide sequence portion
ggacuggucuuucuaucucu

SEQ ID NO: 466 - RNA, 20 nt, Artificial Sequence
  B2M-targeting guide sequence portion
ggagagagaauugaaaaagu

SEQ ID NO: 467 - RNA, 20 nt, Artificial Sequence
  B2M-targeting guide sequence portion
ggagcauucagacuugucuu

SEQ ID NO: 468 - RNA, 20 nt, Artificial Sequence
  B2M-targeting guide sequence portion
ggaugaaacccagacacaua

SEQ ID NO: 469 - RNA, 20 nt, Artificial Sequence
  B2M-targeting guide sequence portion
ggaugacgugaguaaaccug

SEQ ID NO: 470 - RNA, 20 nt, Artificial Sequence
  B2M-targeting guide sequence portion
ggauggaugaaacccagaca

SEQ ID NO: 471 - RNA, 20 nt, Artificial Sequence
  B2M-targeting guide sequence portion
gggggugaauucaguguagu

SEQ ID NO: 472 - RNA, 20 nt, Artificial Sequence
  B2M-targeting guide sequence portion
ggggugaauucaguguagua

SEQ ID NO: 473 - RNA, 20 nt, Artificial Sequence
  B2M-targeting guide sequence portion
ggguuucauccauccgacau

SEQ ID NO: 474 - RNA, 20 nt, Artificial Sequence
  B2M-targeting guide sequence portion
gguacuccaaagauucaggu

SEQ ID NO: 475 - RNA, 20 nt, Artificial Sequence
  B2M-targeting guide sequence portion
ggucuuucuaucucuuguac

SEQ ID NO: 476 - RNA, 20 nt, Artificial Sequence
  B2M-targeting guide sequence portion
ggugaauucaguguaguaca

SEQ ID NO: 477 - RNA, 20 nt, Artificial Sequence
  B2M-targeting guide sequence portion
gguucacacggcaggcauac

SEQ ID NO: 478 - RNA, 20 nt, Artificial Sequence
  B2M-targeting guide sequence portion
guaaaccugaaucuuuggag

SEQ ID NO: 479 - RNA, 20 nt, Artificial Sequence
  B2M-targeting guide sequence portion
guaagucaacuucaaugucg

SEQ ID NO: 480 - RNA, 20 nt, Artificial Sequence
  B2M-targeting guide sequence portion
guacaagagauagaaagacc

SEQ ID NO: 481 - RNA, 20 nt, Artificial Sequence
  B2M-targeting guide sequence portion
guacuccaaagauucagguu

SEQ ID NO: 482 - RNA, 20 nt, Artificial Sequence
  B2M-targeting guide sequence portion
guaguacaagagauagaaag

SEQ ID NO: 483 - RNA, 20 nt, Artificial Sequence
  B2M-targeting guide sequence portion
gucaaauuuccugaauugcu

SEQ ID NO: 484 - RNA, 20 nt, Artificial Sequence
  B2M-targeting guide sequence portion
gucaacuucaaugucggaug

SEQ ID NO: 485 - RNA, 20 nt, Artificial Sequence
  B2M-targeting guide sequence portion
gucacaugguucacacggca

SEQ ID NO: 486 - RNA, 20 nt, Artificial Sequence
  B2M-targeting guide sequence portion
gucggauggaugaaacccag

SEQ ID NO: 487 - RNA, 20 nt, Artificial Sequence
  B2M-targeting guide sequence portion
gucugaaugcuccacuuuuu

SEQ ID NO: 488 - RNA, 20 nt, Artificial Sequence
  B2M-targeting guide sequence portion
gugaaccaugugacuuuguc

SEQ ID NO: 489 - RNA, 20 nt, Artificial Sequence
  B2M-targeting guide sequence portion
gugacaaagucacaugguuc

SEQ ID NO: 490 - RNA, 20 nt, Artificial Sequence
  B2M-targeting guide sequence portion
gugacuuugucacagcccaa

SEQ ID NO: 491 - RNA, 20 nt, Artificial Sequence
  B2M-targeting guide sequence portion
gugaguaaaccugaaucuuu

SEQ ID NO: 492 - RNA, 20 nt, Artificial Sequence
  B2M-targeting guide sequence portion
guggagcauucagacuuguc

SEQ ID NO: 493 - RNA, 20 nt, Artificial Sequence
  B2M-targeting guide sequence portion
guguaguacaagagauagaa

SEQ ID NO: 494 - RNA, 20 nt, Artificial Sequence
  B2M-targeting guide sequence portion
gugugaaccaugugacuuug

SEQ ID NO: 495 - RNA, 20 nt, Artificial Sequence
  B2M-targeting guide sequence portion
guucacacggcaggcauacu

SEQ ID NO: 496 - RNA, 20 nt, Artificial Sequence
  B2M-targeting guide sequence portion
guugacuuacugaagaaugg

SEQ ID NO: 497 - RNA, 20 nt, Artificial Sequence
  B2M-targeting guide sequence portion
uaaaccugaaucuuuggagu

SEQ ID NO: 498 - RNA, 20 nt, Artificial Sequence
  B2M-targeting guide sequence portion
uaacuaucuugggcugugac

SEQ ID NO: 499 - RNA, 20 nt, Artificial Sequence
  B2M-targeting guide sequence portion
uacaagagauagaaagacca

SEQ ID NO: 500 - RNA, 20 nt, Artificial Sequence
  B2M-targeting guide sequence portion
uacacugaauucacccccac

SEQ ID NO: 501 - RNA, 20 nt, Artificial Sequence
  B2M-targeting guide sequence portion
uaccccacuuaacuaucuug

SEQ ID NO: 502 - RNA, 20 nt, Artificial Sequence
  B2M-targeting guide sequence portion
uacucacgucauccagcaga

SEQ ID NO: 503 - RNA, 20 nt, Artificial Sequence
  B2M-targeting guide sequence portion
uacucaucuuuuucaguggg

SEQ ID NO: 504 - RNA, 20 nt, Artificial Sequence
  B2M-targeting guide sequence portion
uacuccaaagauucagguuu

SEQ ID NO: 505 - RNA, 20 nt, Artificial Sequence
  B2M-targeting guide sequence portion
uacugaagaauggagagaga

SEQ ID NO: 506 - RNA, 20 nt, Artificial Sequence
  B2M-targeting guide sequence portion
uagaaagaccaguccuugcu

SEQ ID NO: 507 - RNA, 20 nt, Artificial Sequence
  B2M-targeting guide sequence portion
uagcaauucaggaaauuuga

SEQ ID NO: 508 - RNA, 20 nt, Artificial Sequence
  B2M-targeting guide sequence portion
uaguacaagagauagaaaga

SEQ ID NO: 509 - RNA, 20 nt, Artificial Sequence
  B2M-targeting guide sequence portion
uaucuugggcugugacaaag

SEQ ID NO: 510 - RNA, 20 nt, Artificial Sequence
  B2M-targeting guide sequence portion
uaugccugccgugugaacca

SEQ ID NO: 511 - RNA, 20 nt, Artificial Sequence
  B2M-targeting guide sequence portion
uaugugucuggguuucaucc

SEQ ID NO: 512 - RNA, 20 nt, Artificial Sequence
  B2M-targeting guide sequence portion
ucaaauuuccugaauugcua

SEQ ID NO: 513 - RNA, 20 nt, Artificial Sequence
  B2M-targeting guide sequence portion
ucaacuucaaugucggaugg

SEQ ID NO: 514 - RNA, 20 nt, Artificial Sequence
  B2M-targeting guide sequence portion
ucacacggcaggcauacuca

SEQ ID NO: 515 - RNA, 20 nt, Artificial Sequence
  B2M-targeting guide sequence portion
ucagacuugucuuucagcaa

SEQ ID NO: 516 - RNA, 20 nt, Artificial Sequence
  B2M-targeting guide sequence portion
ucaggaaauuugacuuucca

SEQ ID NO: 517 - RNA, 20 nt, Artificial Sequence
  B2M-targeting guide sequence portion
ucagguacuccaaagauuca

SEQ ID NO: 518 - RNA, 20 nt, Artificial Sequence
  B2M-targeting guide sequence portion
ucaguguaguacaagagaua

SEQ ID NO: 519 - RNA, 20 nt, Artificial Sequence
  B2M-targeting guide sequence portion
ucauccagcagagaauggaa

SEQ ID NO: 520 - RNA, 20 nt, Artificial Sequence
  B2M-targeting guide sequence portion
ucauccauccgacauugaag

SEQ ID NO: 521 - RNA, 20 nt, Artificial Sequence
  B2M-targeting guide sequence portion
ucaucuuuuucagugggggu

SEQ ID NO: 522 - RNA, 20 nt, Artificial Sequence
  B2M-targeting guide sequence portion
uccaaagauucagguuuacu

SEQ ID NO: 523 - RNA, 20 nt, Artificial Sequence
  B2M-targeting guide sequence portion
uccacuuuuucaauucucuc

SEQ ID NO: 524 - RNA, 20 nt, Artificial Sequence
  B2M-targeting guide sequence portion
uccagcagagaauggaaagu

SEQ ID NO: 525 - RNA, 20 nt, Artificial Sequence
  B2M-targeting guide sequence portion
uccauccgacauugaaguug

SEQ ID NO: 526 - RNA, 20 nt, Artificial Sequence
  B2M-targeting guide sequence portion
uccgacauugaaguugacuu

SEQ ID NO: 527 - RNA, 20 nt, Artificial Sequence
  B2M-targeting guide sequence portion
uccuugcugaaagacaaguc

SEQ ID NO: 528 - RNA, 20 nt, Artificial Sequence
  B2M-targeting guide sequence portion
ucggauggaugaaacccaga

SEQ ID NO: 529 - RNA, 20 nt, Artificial Sequence
  B2M-targeting guide sequence portion
ucucucuccauucuucagua

SEQ ID NO: 530 - RNA, 20 nt, Artificial Sequence
  B2M-targeting guide sequence portion
ucugaaugcuccacuuuuuc

SEQ ID NO: 531 - RNA, 20 nt, Artificial Sequence
  B2M-targeting guide sequence portion
ucuggguuucauccauccga

SEQ ID NO: 532 - RNA, 20 nt, Artificial Sequence
  B2M-targeting guide sequence portion
ucuucaguaagucaacuuca

SEQ ID NO: 533 - RNA, 20 nt, Artificial Sequence
  B2M-targeting guide sequence portion
ucuuguacuacacugaauuc

SEQ ID NO: 534 - RNA, 20 nt, Artificial Sequence
  B2M-targeting guide sequence portion
ucuuucuaucucuuguacua

SEQ ID NO: 535 - RNA, 20 nt, Artificial Sequence
  B2M-targeting guide sequence portion
ucuuuuucagugggggugaa

SEQ ID NO: 536 - RNA, 20 nt, Artificial Sequence
  B2M-targeting guide sequence portion
ugaaaaagaugaguaugccu

SEQ ID NO: 537 - RNA, 20 nt, Artificial Sequence
  B2M-targeting guide sequence portion
ugaaaaaguggagcauucag

SEQ ID NO: 538 - RNA, 20 nt, Artificial Sequence
  B2M-targeting guide sequence portion
ugaaacccagacacauagca

SEQ ID NO: 539 - RNA, 20 nt, Artificial Sequence
  B2M-targeting guide sequence portion
ugaaagacaagucugaaugc

SEQ ID NO: 540 - RNA, 20 nt, Artificial Sequence
  B2M-targeting guide sequence portion
ugaagaauggagagagaauu

SEQ ID NO: 541 - RNA, 20 nt, Artificial Sequence
  B2M-targeting guide sequence portion
ugaaguugacuuacugaaga

SEQ ID NO: 542 - RNA, 20 nt, Artificial Sequence
  B2M-targeting guide sequence portion
ugaaugcuccacuuuuucaa

SEQ ID NO: 543 - RNA, 20 nt, Artificial Sequence
  B2M-targeting guide sequence portion
ugaauucaguguaguacaag

SEQ ID NO: 544 - RNA, 20 nt, Artificial Sequence
  B2M-targeting guide sequence portion
ugaauugcuaugugucuggg

SEQ ID NO: 545 - RNA, 20 nt, Artificial Sequence
  B2M-targeting guide sequence portion
ugacgugaguaaaccugaau

SEQ ID NO: 546 - RNA, 20 nt, Artificial Sequence
  B2M-targeting guide sequence portion
ugacuuuccauucucugcug

SEQ ID NO: 547 - RNA, 20 nt, Artificial Sequence
  B2M-targeting guide sequence portion
ugccgugugaaccaugugac

SEQ ID NO: 548 - RNA, 20 nt, Artificial Sequence
  B2M-targeting guide sequence portion
ugccugccgugugaaccaug

SEQ ID NO: 549 - RNA, 20 nt, Artificial Sequence
  B2M-targeting guide sequence portion
ugcuaugugucuggguuuca

SEQ ID NO: 550 - RNA, 20 nt, Artificial Sequence
  B2M-targeting guide sequence portion
ugcuccacuuuuucaauucu

SEQ ID NO: 551 - RNA, 20 nt, Artificial Sequence
  B2M-targeting guide sequence portion
ugcugaaagacaagucugaa

SEQ ID NO: 552 - RNA, 20 nt, Artificial Sequence
  B2M-targeting guide sequence portion
uggaaagucaaauuuccuga

SEQ ID NO: 553 - RNA, 20 nt, Artificial Sequence
  B2M-targeting guide sequence portion
uggagcauucagacuugucu

SEQ ID NO: 554 - RNA, 20 nt, Artificial Sequence
  B2M-targeting guide sequence portion
uggaugaaacccagacacau

SEQ ID NO: 555 - RNA, 20 nt, Artificial Sequence
  B2M-targeting guide sequence portion
uggaugacgugaguaaaccu

SEQ ID NO: 556 - RNA, 20 nt, Artificial Sequence
  B2M-targeting guide sequence portion
ugggggugaauucaguguag

SEQ ID NO: 557 - RNA, 20 nt, Artificial Sequence
  B2M-targeting guide sequence portion
uggguuucauccauccgaca

SEQ ID NO: 558 - RNA, 20 nt, Artificial Sequence
  B2M-targeting guide sequence portion
uggucuuucuaucucuugua

SEQ ID NO: 559 - RNA, 20 nt, Artificial Sequence
  B2M-targeting guide sequence portion
ugguucacacggcaggcaua

SEQ ID NO: 560 - RNA, 20 nt, Artificial Sequence
  B2M-targeting guide sequence portion
uguacuacacugaauucacc

SEQ ID NO: 561 - RNA, 20 nt, Artificial Sequence
  B2M-targeting guide sequence portion
uguaguacaagagauagaaa

SEQ ID NO: 562 - RNA, 20 nt, Artificial Sequence
  B2M-targeting guide sequence portion
ugucuggguuucauccaucc

SEQ ID NO: 563 - RNA, 20 nt, Artificial Sequence
  B2M-targeting guide sequence portion
ugucuuucagcaaggacugg

SEQ ID NO: 564 - RNA, 20 nt, Artificial Sequence
  B2M-targeting guide sequence portion
ugugaaccaugugacuuugu

SEQ ID NO: 565 - RNA, 20 nt, Artificial Sequence
  B2M-targeting guide sequence portion
ugugacaaagucacaugguu

SEQ ID NO: 566 - RNA, 20 nt, Artificial Sequence
  B2M-targeting guide sequence portion
ugugacuuugucacagccca

SEQ ID NO: 567 - RNA, 20 nt, Artificial Sequence
  B2M-targeting guide sequence portion
uuaacuaucuugggcuguga

SEQ ID NO: 568 - RNA, 20 nt, Artificial Sequence
  B2M-targeting guide sequence portion
uuacucacgucauccagcag

SEQ ID NO: 569 - RNA, 20 nt, Artificial Sequence
  B2M-targeting guide sequence portion
uuacugaagaauggagagag

SEQ ID NO: 570 - RNA, 20 nt, Artificial Sequence
  B2M-targeting guide sequence portion
uucacacggcaggcauacuc

SEQ ID NO: 571 - RNA, 20 nt, Artificial Sequence
  B2M-targeting guide sequence portion
uucacccccacugaaaaaga

SEQ ID NO: 572 - RNA, 20 nt, Artificial Sequence
  B2M-targeting guide sequence portion
uucagcaaggacuggucuuu

SEQ ID NO: 573 - RNA, 20 nt, Artificial Sequence
  B2M-targeting guide sequence portion
uucaggaaauuugacuuucc

SEQ ID NO: 574 - RNA, 20 nt, Artificial Sequence
  B2M-targeting guide sequence portion
uucagguuuacucacgucau

SEQ ID NO: 575 - RNA, 20 nt, Artificial Sequence
  B2M-targeting guide sequence portion
uucaguguaguacaagagau

SEQ ID NO: 576 - RNA, 20 nt, Artificial Sequence
  B2M-targeting guide sequence portion
uucauccauccgacauugaa

SEQ ID NO: 577 - RNA, 20 nt, Artificial Sequence
  B2M-targeting guide sequence portion
uuccauucucugcuggauga

SEQ ID NO: 578 - RNA, 20 nt, Artificial Sequence
  B2M-targeting guide sequence portion
uuccucagguacuccaaaga

SEQ ID NO: 579 - RNA, 20 nt, Artificial Sequence
  B2M-targeting guide sequence portion
uucuaucucuuguacuacac

SEQ ID NO: 580 - RNA, 20 nt, Artificial Sequence
  B2M-targeting guide sequence portion
uucucugcuggaugacguga

SEQ ID NO: 581 - RNA, 20 nt, Artificial Sequence
  B2M-targeting guide sequence portion
uucuucaguaagucaacuuc

SEQ ID NO: 582 - RNA, 20 nt, Artificial Sequence
  B2M-targeting guide sequence portion
uugaaaaaguggagcauuca

SEQ ID NO: 583 - RNA, 20 nt, Artificial Sequence
  B2M-targeting guide sequence portion
uugaaguugacuuacugaag

SEQ ID NO: 584 - RNA, 20 nt, Artificial Sequence
  B2M-targeting guide sequence portion
uugacuuuccauucucugcu

SEQ ID NO: 585 - RNA, 20 nt, Artificial Sequence
  B2M-targeting guide sequence portion
uugcuaugugucuggguuuc

SEQ ID NO: 586 - RNA, 20 nt, Artificial Sequence
  B2M-targeting guide sequence portion
uugcugaaagacaagucuga

SEQ ID NO: 587 - RNA, 20 nt, Artificial Sequence
  B2M-targeting guide sequence portion
uugggcugugacaaagucac

SEQ ID NO: 588 - RNA, 20 nt, Artificial Sequence
  B2M-targeting guide sequence portion
uugucacagcccaagauagu

SEQ ID NO: 589 - RNA, 20 nt, Artificial Sequence
  B2M-targeting guide sequence portion
uugucuuucagcaaggacug

SEQ ID NO: 590 - RNA, 20 nt, Artificial Sequence
  B2M-targeting guide sequence portion
uuucagcaaggacuggucuu

SEQ ID NO: 591 - RNA, 20 nt, Artificial Sequence
  B2M-targeting guide sequence portion
uuuccauucucugcuggaug

SEQ ID NO: 592 - RNA, 20 nt, Artificial Sequence
  B2M-targeting guide sequence portion
uuugucacagcccaagauag

SEQ ID NO: 593 - RNA, 20 nt, Artificial Sequence
  B2M-targeting guide sequence portion
uuuucaauucucucuccauu

SEQ ID NO: 594 - RNA, 20 nt, Artificial Sequence
  B2M-targeting guide sequence portion
uuuucagugggggugaauuc

SEQ ID NO: 595 - RNA, 20 nt, Artificial Sequence
  B2M-targeting guide sequence portion
uuuuucaauucucucuccau

SEQ ID NO: 596 - RNA, 21 nt, Artificial Sequence
  B2M-targeting guide sequence portion
aaaaagaugaguaugccugcc

SEQ ID NO: 597 - RNA, 21 nt, Artificial Sequence
  B2M-targeting guide sequence portion
aaaagaugaguaugccugccg

SEQ ID NO: 598 - RNA, 21 nt, Artificial Sequence
  B2M-targeting guide sequence portion
aaaaguggagcauucagacuu

SEQ ID NO: 599 - RNA, 21 nt, Artificial Sequence
  B2M-targeting guide sequence portion
aaacccagacacauagcaauu

SEQ ID NO: 600 - RNA, 21 nt, Artificial Sequence
  B2M-targeting guide sequence portion
aaaccugaaucuuuggaguac

SEQ ID NO: 601 - RNA, 21 nt, Artificial Sequence
  B2M-targeting guide sequence portion
aaagacaagucugaaugcucc

SEQ ID NO: 602 - RNA, 21 nt, Artificial Sequence
  B2M-targeting guide sequence portion
aaagaccaguccuugcugaaa

SEQ ID NO: 603 - RNA, 21 nt, Artificial Sequence
  B2M-targeting guide sequence portion
aaagaugaguaugccugccgu

SEQ ID NO: 604 - RNA, 21 nt, Artificial Sequence
  B2M-targeting guide sequence portion
aaagauucagguuuacucacg

SEQ ID NO: 605 - RNA, 21 nt, Artificial Sequence
  B2M-targeting guide sequence portion
aaagucacaugguucacacgg

SEQ ID NO: 606 - RNA, 21 nt, Artificial Sequence
  B2M-targeting guide sequence portion
aaaguggagcauucagacuug

SEQ ID NO: 607 - RNA, 21 nt, Artificial Sequence
  B2M-targeting guide sequence portion
aaauuuccugaauugcuaugu

SEQ ID NO: 608 - RNA, 21 nt, Artificial Sequence
  B2M-targeting guide sequence portion
aaauuugacuuuccauucucu

SEQ ID NO: 609 - RNA, 21 nt, Artificial Sequence
  B2M-targeting guide sequence portion
aacccagacacauagcaauuc

SEQ ID NO: 610 - RNA, 21 nt, Artificial Sequence
  B2M-targeting guide sequence portion
aaccugaaucuuuggaguacc

SEQ ID NO: 611 - RNA, 21 nt, Artificial Sequence
  B2M-targeting guide sequence portion
aacuaucuugggcugugacaa

SEQ ID NO: 612 - RNA, 21 nt, Artificial Sequence
  B2M-targeting guide sequence portion
aacuucaaugucggauggaug

SEQ ID NO: 613 - RNA, 21 nt, Artificial Sequence
  B2M-targeting guide sequence portion
aagaauggagagagaauugaa

SEQ ID NO: 614 - RNA, 21 nt, Artificial Sequence
  B2M-targeting guide sequence portion
aagacaagucugaaugcucca

SEQ ID NO: 615 - RNA, 21 nt, Artificial Sequence
  B2M-targeting guide sequence portion
aagaccaguccuugcugaaag

SEQ ID NO: 616 - RNA, 21 nt, Artificial Sequence
  B2M-targeting guide sequence portion
aagagauagaaagaccagucc

SEQ ID NO: 617 - RNA, 21 nt, Artificial Sequence
  B2M-targeting guide sequence portion
aagauucagguuuacucacgu

SEQ ID NO: 618 - RNA, 21 nt, Artificial Sequence
  B2M-targeting guide sequence portion
aaggacuggucuuucuaucuc

SEQ ID NO: 619 - RNA, 21 nt, Artificial Sequence
  B2M-targeting guide sequence portion
aagucaaauuuccugaauugc

SEQ ID NO: 620 - RNA, 21 nt, Artificial Sequence
  B2M-targeting guide sequence portion
aagucaacuucaaugucggau

SEQ ID NO: 621 - RNA, 21 nt, Artificial Sequence
  B2M-targeting guide sequence portion
aagucacaugguucacacggc

SEQ ID NO: 622 - RNA, 21 nt, Artificial Sequence
  B2M-targeting guide sequence portion
aagucugaaugcuccacuuuu

SEQ ID NO: 623 - RNA, 21 nt, Artificial Sequence
  B2M-targeting guide sequence portion
aaguggagcauucagacuugu

SEQ ID NO: 624 - RNA, 21 nt, Artificial Sequence
  B2M-targeting guide sequence portion
aaguugacuuacugaagaaug

SEQ ID NO: 625 - RNA, 21 nt, Artificial Sequence
  B2M-targeting guide sequence portion
aaugcuccacuuuuucaauuc

SEQ ID NO: 626 - RNA, 21 nt, Artificial Sequence
  B2M-targeting guide sequence portion
aauggaaagucaaauuuccug

SEQ ID NO: 627 - RNA, 21 nt, Artificial Sequence
  B2M-targeting guide sequence portion
aauggagagagaauugaaaaa

SEQ ID NO: 628 - RNA, 21 nt, Artificial Sequence
  B2M-targeting guide sequence portion
aaugucggauggaugaaaccc

SEQ ID NO: 629 - RNA, 21 nt, Artificial Sequence
  B2M-targeting guide sequence portion
aauucacccccacugaaaaag

SEQ ID NO: 630 - RNA, 21 nt, Artificial Sequence
  B2M-targeting guide sequence portion
aauugaaaaaguggagcauuc

SEQ ID NO: 631 - RNA, 21 nt, Artificial Sequence
  B2M-targeting guide sequence portion
aauugcuaugugucuggguuu

SEQ ID NO: 632 - RNA, 21 nt, Artificial Sequence
  B2M-targeting guide sequence portion
aauuuccugaauugcuaugug

SEQ ID NO: 633 - RNA, 21 nt, Artificial Sequence
  B2M-targeting guide sequence portion
aauuugacuuuccauucucug

SEQ ID NO: 634 - RNA, 21 nt, Artificial Sequence
  B2M-targeting guide sequence portion
acaaagucacaugguucacac

SEQ ID NO: 635 - RNA, 21 nt, Artificial Sequence
  B2M-targeting guide sequence portion
acaagagauagaaagaccagu

SEQ ID NO: 636 - RNA, 21 nt, Artificial Sequence
  B2M-targeting guide sequence portion
acaagucugaaugcuccacuu

SEQ ID NO: 637 - RNA, 21 nt, Artificial Sequence
  B2M-targeting guide sequence portion
acacauagcaauucaggaaau

SEQ ID NO: 638 - RNA, 21 nt, Artificial Sequence
  B2M-targeting guide sequence portion
acacggcaggcauacucaucu

SEQ ID NO: 639 - RNA, 21 nt, Artificial Sequence
  B2M-targeting guide sequence portion
acacugaauucacccccacug

SEQ ID NO: 640 - RNA, 21 nt, Artificial Sequence
  B2M-targeting guide sequence portion
acagcccaagauaguuaagug

SEQ ID NO: 641 - RNA, 21 nt, Artificial Sequence
  B2M-targeting guide sequence portion
acaugguucacacggcaggca

SEQ ID NO: 642 - RNA, 21 nt, Artificial Sequence
  B2M-targeting guide sequence portion
acauugaaguugacuuacuga

SEQ ID NO: 643 - RNA, 21 nt, Artificial Sequence
  B2M-targeting guide sequence portion
accaguccuugcugaaagaca

SEQ ID NO: 644 - RNA, 21 nt, Artificial Sequence
  B2M-targeting guide sequence portion
accaugugacuuugucacagc

SEQ ID NO: 645 - RNA, 21 nt, Artificial Sequence
  B2M-targeting guide sequence portion
acccagacacauagcaauuca

SEQ ID NO: 646 - RNA, 21 nt, Artificial Sequence
  B2M-targeting guide sequence portion
accccacuuaacuaucuuggg

SEQ ID NO: 647 - RNA, 21 nt, Artificial Sequence
  B2M-targeting guide sequence portion
acccccacugaaaaagaugag

SEQ ID NO: 648 - RNA, 21 nt, Artificial Sequence
  B2M-targeting guide sequence portion
accugaaucuuuggaguaccu

SEQ ID NO: 649 - RNA, 21 nt, Artificial Sequence
  B2M-targeting guide sequence portion
acggcaggcauacucaucuuu

SEQ ID NO: 650 - RNA, 21 nt, Artificial Sequence
  B2M-targeting guide sequence portion
acgucauccagcagagaaugg

SEQ ID NO: 651 - RNA, 21 nt, Artificial Sequence
  B2M-targeting guide sequence portion
acuacacugaauucaccccca

SEQ ID NO: 652 - RNA, 21 nt, Artificial Sequence
  B2M-targeting guide sequence portion
acuaucuugggcugugacaaa

SEQ ID NO: 653 - RNA, 21 nt, Artificial Sequence
  B2M-targeting guide sequence portion
acucacgucauccagcagaga

SEQ ID NO: 654 - RNA, 21 nt, Artificial Sequence
  B2M-targeting guide sequence portion
acucaucuuuuucaguggggg

SEQ ID NO: 655 - RNA, 21 nt, Artificial Sequence
  B2M-targeting guide sequence portion
acuccaaagauucagguuuac

SEQ ID NO: 656 - RNA, 21 nt, Artificial Sequence
  B2M-targeting guide sequence portion
acugaaaaagaugaguaugcc

SEQ ID NO: 657 - RNA, 21 nt, Artificial Sequence
  B2M-targeting guide sequence portion
acugaagaauggagagagaau

SEQ ID NO: 658 - RNA, 21 nt, Artificial Sequence
  B2M-targeting guide sequence portion
acugaauucacccccacugaa

SEQ ID NO: 659 - RNA, 21 nt, Artificial Sequence
  B2M-targeting guide sequence portion
acuggucuuucuaucucuugu

SEQ ID NO: 660 - RNA, 21 nt, Artificial Sequence
  B2M-targeting guide sequence portion
acuuaacuaucuugggcugug

SEQ ID NO: 661 - RNA, 21 nt, Artificial Sequence
  B2M-targeting guide sequence portion
acuuaccccacuuaacuaucu

SEQ ID NO: 662 - RNA, 21 nt, Artificial Sequence
  B2M-targeting guide sequence portion
acuuacugaagaauggagaga

SEQ ID NO: 663 - RNA, 21 nt, Artificial Sequence
  B2M-targeting guide sequence portion
acuucaaugucggauggauga

SEQ ID NO: 664 - RNA, 21 nt, Artificial Sequence
  B2M-targeting guide sequence portion
acuugucuuucagcaaggacu

SEQ ID NO: 665 - RNA, 21 nt, Artificial Sequence
  B2M-targeting guide sequence portion
acuuugucacagcccaagaua

SEQ ID NO: 666 - RNA, 21 nt, Artificial Sequence
  B2M-targeting guide sequence portion
acuuuuucaauucucucucca

SEQ ID NO: 667 - RNA, 21 nt, Artificial Sequence
  B2M-targeting guide sequence portion
agaaagaccaguccuugcuga

SEQ ID NO: 668 - RNA, 21 nt, Artificial Sequence
  B2M-targeting guide sequence portion
agaauggagagagaauugaaa

SEQ ID NO: 669 - RNA, 21 nt, Artificial Sequence
  B2M-targeting guide sequence portion
agaauugaaaaaguggagcau

SEQ ID NO: 670 - RNA, 21 nt, Artificial Sequence
  B2M-targeting guide sequence portion
agacaagucugaaugcuccac

SEQ ID NO: 671 - RNA, 21 nt, Artificial Sequence
  B2M-targeting guide sequence portion
agacacauagcaauucaggaa

SEQ ID NO: 672 - RNA, 21 nt, Artificial Sequence
  B2M-targeting guide sequence portion
agaccaguccuugcugaaaga

SEQ ID NO: 673 - RNA, 21 nt, Artificial Sequence
  B2M-targeting guide sequence portion
agacuugucuuucagcaagga

SEQ ID NO: 674 - RNA, 21 nt, Artificial Sequence
  B2M-targeting guide sequence portion
agagaauggaaagucaaauuu

SEQ ID NO: 675 - RNA, 21 nt, Artificial Sequence
  B2M-targeting guide sequence portion
agagaauugaaaaaguggagc

SEQ ID NO: 676 - RNA, 21 nt, Artificial Sequence
  B2M-targeting guide sequence portion
agagagaauugaaaaagugga

SEQ ID NO: 677 - RNA, 21 nt, Artificial Sequence
  B2M-targeting guide sequence portion
agagauagaaagaccaguccu

SEQ ID NO: 678 - RNA, 21 nt, Artificial Sequence
  B2M-targeting guide sequence portion
agauagaaagaccaguccuug

SEQ ID NO: 679 - RNA, 21 nt, Artificial Sequence
  B2M-targeting guide sequence portion
agaugaguaugccugccgugu

SEQ ID NO: 680 - RNA, 21 nt, Artificial Sequence
  B2M-targeting guide sequence portion
agauucagguuuacucacguc

SEQ ID NO: 681 - RNA, 21 nt, Artificial Sequence
  B2M-targeting guide sequence portion
agcaauucaggaaauuugacu

SEQ ID NO: 682 - RNA, 21 nt, Artificial Sequence
  B2M-targeting guide sequence portion
agcauucagacuugucuuuca

SEQ ID NO: 683 - RNA, 21 nt, Artificial Sequence
  B2M-targeting guide sequence portion
agcccaagauaguuaaguggg

SEQ ID NO: 684 - RNA, 21 nt, Artificial Sequence
  B2M-targeting guide sequence portion
aggaaauuugacuuuccauuc

SEQ ID NO: 685 - RNA, 21 nt, Artificial Sequence
  B2M-targeting guide sequence portion
aggcauacucaucuuuuucag

SEQ ID NO: 686 - RNA, 21 nt, Artificial Sequence
  B2M-targeting guide sequence portion
agguacuccaaagauucaggu

SEQ ID NO: 687 - RNA, 21 nt, Artificial Sequence
  B2M-targeting guide sequence portion
agguuuacucacgucauccag

SEQ ID NO: 688 - RNA, 21 nt, Artificial Sequence
  B2M-targeting guide sequence portion
aguaaaccugaaucuuuggag

SEQ ID NO: 689 - RNA, 21 nt, Artificial Sequence
  B2M-targeting guide sequence portion
aguaagucaacuucaaugucg

SEQ ID NO: 690 - RNA, 21 nt, Artificial Sequence
  B2M-targeting guide sequence portion
aguacaagagauagaaagacc

SEQ ID NO: 691 - RNA, 21 nt, Artificial Sequence
  B2M-targeting guide sequence portion
aguaugccugccgugugaacc

SEQ ID NO: 692 - RNA, 21 nt, Artificial Sequence
  B2M-targeting guide sequence portion
agucaaauuuccugaauugcu

SEQ ID NO: 693 - RNA, 21 nt, Artificial Sequence
  B2M-targeting guide sequence portion
agucaacuucaaugucggaug

SEQ ID NO: 694 - RNA, 21 nt, Artificial Sequence
  B2M-targeting guide sequence portion
agucacaugguucacacggca

SEQ ID NO: 695 - RNA, 21 nt, Artificial Sequence
  B2M-targeting guide sequence portion
aguccuugcugaaagacaagu

SEQ ID NO: 696 - RNA, 21 nt, Artificial Sequence
  B2M-targeting guide sequence portion
aguggagcauucagacuuguc

SEQ ID NO: 697 - RNA, 21 nt, Artificial Sequence
  B2M-targeting guide sequence portion
aguguaguacaagagauagaa

SEQ ID NO: 698 - RNA, 21 nt, Artificial Sequence
  B2M-targeting guide sequence portion
aguugacuuacugaagaaugg

SEQ ID NO: 699 - RNA, 21 nt, Artificial Sequence
  B2M-targeting guide sequence portion
auagaaagaccaguccuugcu

SEQ ID NO: 700 - RNA, 21 nt, Artificial Sequence
  B2M-targeting guide sequence portion
auccagcagagaauggaaagu

SEQ ID NO: 701 - RNA, 21 nt, Artificial Sequence
  B2M-targeting guide sequence portion
aucucuuguacuacacugaau

SEQ ID NO: 702 - RNA, 21 nt, Artificial Sequence
  B2M-targeting guide sequence portion
aucuugggcugugacaaaguc

SEQ ID NO: 703 - RNA, 21 nt, Artificial Sequence
  B2M-targeting guide sequence portion
aucuuuuucagugggggugaa

SEQ ID NO: 704 - RNA, 21 nt, Artificial Sequence
  B2M-targeting guide sequence portion
augaaacccagacacauagca

SEQ ID NO: 705 - RNA, 21 nt, Artificial Sequence
  B2M-targeting guide sequence portion
augccugccgugugaaccaug

SEQ ID NO: 706 - RNA, 21 nt, Artificial Sequence
  B2M-targeting guide sequence portion
auggaaagucaaauuuccuga

SEQ ID NO: 707 - RNA, 21 nt, Artificial Sequence
  B2M-targeting guide sequence portion
auggaugaaacccagacacau

SEQ ID NO: 708 - RNA, 21 nt, Artificial Sequence
  B2M-targeting guide sequence portion
augguucacacggcaggcaua

SEQ ID NO: 709 - RNA, 21 nt, Artificial Sequence
  B2M-targeting guide sequence portion
augucggauggaugaaaccca

SEQ ID NO: 710 - RNA, 21 nt, Artificial Sequence
  B2M-targeting guide sequence portion
auucagacuugucuuucagca

SEQ ID NO: 711 - RNA, 21 nt, Artificial Sequence
  B2M-targeting guide sequence portion
auucaggaaauuugacuuucc

SEQ ID NO: 712 - RNA, 21 nt, Artificial Sequence
  B2M-targeting guide sequence portion
auucagguuuacucacgucau

SEQ ID NO: 713 - RNA, 21 nt, Artificial Sequence
  B2M-targeting guide sequence portion
auucucucuccauucuucagu

SEQ ID NO: 714 - RNA, 21 nt, Artificial Sequence
  B2M-targeting guide sequence portion
auugaaaaaguggagcauuca

SEQ ID NO: 715 - RNA, 21 nt, Artificial Sequence
  B2M-targeting guide sequence portion
auugaaguugacuuacugaag

SEQ ID NO: 716 - RNA, 21 nt, Artificial Sequence
  B2M-targeting guide sequence portion
auugcuaugugucuggguuuc

SEQ ID NO: 717 - RNA, 21 nt, Artificial Sequence
  B2M-targeting guide sequence portion
auuuccugaauugcuaugugu

SEQ ID NO: 718 - RNA, 21 nt, Artificial Sequence
  B2M-targeting guide sequence portion
auuugacuuuccauucucugc

SEQ ID NO: 719 - RNA, 21 nt, Artificial Sequence
  B2M-targeting guide sequence portion
caaagauucagguuuacucac

SEQ ID NO: 720 - RNA, 21 nt, Artificial Sequence
  B2M-targeting guide sequence portion
caaagucacaugguucacacg

SEQ ID NO: 721 - RNA, 21 nt, Artificial Sequence
  B2M-targeting guide sequence portion
caaauuuccugaauugcuaug

SEQ ID NO: 722 - RNA, 21 nt, Artificial Sequence
  B2M-targeting guide sequence portion
caacuucaaugucggauggau

SEQ ID NO: 723 - RNA, 21 nt, Artificial Sequence
  B2M-targeting guide sequence portion
caagagauagaaagaccaguc

SEQ ID NO: 724 - RNA, 21 nt, Artificial Sequence
  B2M-targeting guide sequence portion
caaggacuggucuuucuaucu

SEQ ID NO: 725 - RNA, 21 nt, Artificial Sequence
  B2M-targeting guide sequence portion
caagucugaaugcuccacuuu

SEQ ID NO: 726 - RNA, 21 nt, Artificial Sequence
  B2M-targeting guide sequence portion
caaugucggauggaugaaacc

SEQ ID NO: 727 - RNA, 21 nt, Artificial Sequence
  B2M-targeting guide sequence portion
caauucaggaaauuugacuuu

SEQ ID NO: 728 - RNA, 21 nt, Artificial Sequence
  B2M-targeting guide sequence portion
cacagcccaagauaguuaagu

SEQ ID NO: 729 - RNA, 21 nt, Artificial Sequence
  B2M-targeting guide sequence portion
cacauagcaauucaggaaauu

SEQ ID NO: 730 - RNA, 21 nt, Artificial Sequence
  B2M-targeting guide sequence portion
cacaugguucacacggcaggc

SEQ ID NO: 731 - RNA, 21 nt, Artificial Sequence
  B2M-targeting guide sequence portion
cacggcaggcauacucaucuu

SEQ ID NO: 732 - RNA, 21 nt, Artificial Sequence
  B2M-targeting guide sequence portion
cacgucauccagcagagaaug

SEQ ID NO: 733 - RNA, 21 nt, Artificial Sequence
  B2M-targeting guide sequence portion
cacugaaaaagaugaguaugc

SEQ ID NO: 734 - RNA, 21 nt, Artificial Sequence
  B2M-targeting guide sequence portion
cacuuaacuaucuugggcugu

SEQ ID NO: 735 - RNA, 21 nt, Artificial Sequence
  B2M-targeting guide sequence portion
cacuuuuucaauucucucucc

SEQ ID NO: 736 - RNA, 21 nt, Artificial Sequence
  B2M-targeting guide sequence portion
cagacacauagcaauucagga

SEQ ID NO: 737 - RNA, 21 nt, Artificial Sequence
  B2M-targeting guide sequence portion
cagacuugucuuucagcaagg

SEQ ID NO: 738 - RNA, 21 nt, Artificial Sequence
  B2M-targeting guide sequence portion
cagagaauggaaagucaaauu

SEQ ID NO: 739 - RNA, 21 nt, Artificial Sequence
  B2M-targeting guide sequence portion
cagcaaggacuggucuuucua

SEQ ID NO: 740 - RNA, 21 nt, Artificial Sequence
  B2M-targeting guide sequence portion
cagcccaagauaguuaagugg

SEQ ID NO: 741 - RNA, 21 nt, Artificial Sequence
  B2M-targeting guide sequence portion
caggaaauuugacuuuccauu

SEQ ID NO: 742 - RNA, 21 nt, Artificial Sequence
  B2M-targeting guide sequence portion
caggcauacucaucuuuuuca

SEQ ID NO: 743 - RNA, 21 nt, Artificial Sequence
  B2M-targeting guide sequence portion
cagguacuccaaagauucagg

SEQ ID NO: 744 - RNA, 21 nt, Artificial Sequence
  B2M-targeting guide sequence portion
caguaagucaacuucaauguc

SEQ ID NO: 745 - RNA, 21 nt, Artificial Sequence
  B2M-targeting guide sequence portion
caguccuugcugaaagacaag

SEQ ID NO: 746 - RNA, 21 nt, Artificial Sequence
  B2M-targeting guide sequence portion
caguguaguacaagagauaga

SEQ ID NO: 747 - RNA, 21 nt, Artificial Sequence
  B2M-targeting guide sequence portion
cauacucaucuuuuucagugg

SEQ ID NO: 748 - RNA, 21 nt, Artificial Sequence
  B2M-targeting guide sequence portion
cauagcaauucaggaaauuug

SEQ ID NO: 749 - RNA, 21 nt, Artificial Sequence
  B2M-targeting guide sequence portion
cauccagcagagaauggaaag

SEQ ID NO: 750 - RNA, 21 nt, Artificial Sequence
  B2M-targeting guide sequence portion
cauccauccgacauugaaguu

SEQ ID NO: 751 - RNA, 21 nt, Artificial Sequence
  B2M-targeting guide sequence portion
cauccgacauugaaguugacu

SEQ ID NO: 752 - RNA, 21 nt, Artificial Sequence
  B2M-targeting guide sequence portion
caucuuuuucaguggggguga

SEQ ID NO: 753 - RNA, 21 nt, Artificial Sequence
  B2M-targeting guide sequence portion
caugguucacacggcaggcau

SEQ ID NO: 754 - RNA, 21 nt, Artificial Sequence
  B2M-targeting guide sequence portion
caugugacuuugucacagccc

SEQ ID NO: 755 - RNA, 21 nt, Artificial Sequence
  B2M-targeting guide sequence portion
cauucagacuugucuuucagc

SEQ ID NO: 756 - RNA, 21 nt, Artificial Sequence
  B2M-targeting guide sequence portion
cauucuucaguaagucaacuu

SEQ ID NO: 757 - RNA, 21 nt, Artificial Sequence
  B2M-targeting guide sequence portion
cauugaaguugacuuacugaa

SEQ ID NO: 758 - RNA, 21 nt, Artificial Sequence
  B2M-targeting guide sequence portion
ccaaagauucagguuuacuca

SEQ ID NO: 759 - RNA, 21 nt, Artificial Sequence
  B2M-targeting guide sequence portion
ccacugaaaaagaugaguaug

SEQ ID NO: 760 - RNA, 21 nt, Artificial Sequence
  B2M-targeting guide sequence portion
ccacuuaacuaucuugggcug

SEQ ID NO: 761 - RNA, 21 nt, Artificial Sequence
  B2M-targeting guide sequence portion
ccacuuuuucaauucucucuc

SEQ ID NO: 762 - RNA, 21 nt, Artificial Sequence
  B2M-targeting guide sequence portion
ccagacacauagcaauucagg

SEQ ID NO: 763 - RNA, 21 nt, Artificial Sequence
  B2M-targeting guide sequence portion
ccagcagagaauggaaaguca

SEQ ID NO: 764 - RNA, 21 nt, Artificial Sequence
  B2M-targeting guide sequence portion
ccaguccuugcugaaagacaa

SEQ ID NO: 765 - RNA, 21 nt, Artificial Sequence
  B2M-targeting guide sequence portion
ccauccgacauugaaguugac

SEQ ID NO: 766 - RNA, 21 nt, Artificial Sequence
  B2M-targeting guide sequence portion
ccaugugacuuugucacagcc

SEQ ID NO: 767 - RNA, 21 nt, Artificial Sequence
  B2M-targeting guide sequence portion
ccauucucugcuggaugacgu

SEQ ID NO: 768 - RNA, 21 nt, Artificial Sequence
  B2M-targeting guide sequence portion
ccauucuucaguaagucaacu

SEQ ID NO: 769 - RNA, 21 nt, Artificial Sequence
  B2M-targeting guide sequence portion
cccacugaaaaagaugaguau

SEQ ID NO: 770 - RNA, 21 nt, Artificial Sequence
  B2M-targeting guide sequence portion
cccacuuaacuaucuugggcu

SEQ ID NO: 771 - RNA, 21 nt, Artificial Sequence
  B2M-targeting guide sequence portion
cccagacacauagcaauucag

SEQ ID NO: 772 - RNA, 21 nt, Artificial Sequence
  B2M-targeting guide sequence portion
ccccacugaaaaagaugagua

SEQ ID NO: 773 - RNA, 21 nt, Artificial Sequence
  B2M-targeting guide sequence portion
ccccacuuaacuaucuugggc

SEQ ID NO: 774 - RNA, 21 nt, Artificial Sequence
  B2M-targeting guide sequence portion
cccccacugaaaaagaugagu

SEQ ID NO: 775 - RNA, 21 nt, Artificial Sequence
  B2M-targeting guide sequence portion
ccgacauugaaguugacuuac

SEQ ID NO: 776 - RNA, 21 nt, Artificial Sequence
  B2M-targeting guide sequence portion
ccgugugaaccaugugacuuu

SEQ ID NO: 777 - RNA, 21 nt, Artificial Sequence
  B2M-targeting guide sequence portion
ccucagguacuccaaagauuc

SEQ ID NO: 778 - RNA, 21 nt, Artificial Sequence
  B2M-targeting guide sequence portion
ccugaauugcuaugugucugg

SEQ ID NO: 779 - RNA, 21 nt, Artificial Sequence
  B2M-targeting guide sequence portion
ccugccgugugaaccauguga

SEQ ID NO: 780 - RNA, 21 nt, Artificial Sequence
  B2M-targeting guide sequence portion
ccuugcugaaagacaagucug

SEQ ID NO: 781 - RNA, 21 nt, Artificial Sequence
  B2M-targeting guide sequence portion
cgacauugaaguugacuuacu

SEQ ID NO: 782 - RNA, 21 nt, Artificial Sequence
  B2M-targeting guide sequence portion
cgucauccagcagagaaugga

SEQ ID NO: 783 - RNA, 21 nt, Artificial Sequence
  B2M-targeting guide sequence portion
cgugaguaaaccugaaucuuu

SEQ ID NO: 784 - RNA, 21 nt, Artificial Sequence
  B2M-targeting guide sequence portion
cgugugaaccaugugacuuug

SEQ ID NO: 785 - RNA, 21 nt, Artificial Sequence
  B2M-targeting guide sequence portion
cucaucuuuuucagugggggu

SEQ ID NO: 786 - RNA, 21 nt, Artificial Sequence
  B2M-targeting guide sequence portion
cuccacuuuuucaauucucuc

SEQ ID NO: 787 - RNA, 21 nt, Artificial Sequence
  B2M-targeting guide sequence portion
cucuccauucuucaguaaguc

SEQ ID NO: 788 - RNA, 21 nt, Artificial Sequence
  B2M-targeting guide sequence portion
cucucuccauucuucaguaag

SEQ ID NO: 789 - RNA, 21 nt, Artificial Sequence
  B2M-targeting guide sequence portion
cucuuguacuacacugaauuc

SEQ ID NO: 790 - RNA, 21 nt, Artificial Sequence
  B2M-targeting guide sequence portion
cugaaagacaagucugaaugc

SEQ ID NO: 791 - RNA, 21 nt, Artificial Sequence
  B2M-targeting guide sequence portion
cugaagaauggagagagaauu

SEQ ID NO: 792 - RNA, 21 nt, Artificial Sequence
  B2M-targeting guide sequence portion
cugaaugcuccacuuuuucaa

SEQ ID NO: 793 - RNA, 21 nt, Artificial Sequence
  B2M-targeting guide sequence portion
cugaauucacccccacugaaa

SEQ ID NO: 794 - RNA, 21 nt, Artificial Sequence
  B2M-targeting guide sequence portion
cugccgugugaaccaugugac

SEQ ID NO: 795 - RNA, 21 nt, Artificial Sequence
  B2M-targeting guide sequence portion
cuggaugacgugaguaaaccu

SEQ ID NO: 796 - RNA, 21 nt, Artificial Sequence
  B2M-targeting guide sequence portion
cuuaacuaucuugggcuguga

SEQ ID NO: 797 - RNA, 21 nt, Artificial Sequence
  B2M-targeting guide sequence portion
cuuaccccacuuaacuaucuu

SEQ ID NO: 798 - RNA, 21 nt, Artificial Sequence
  B2M-targeting guide sequence portion
cuuacugaagaauggagagag

SEQ ID NO: 799 - RNA, 21 nt, Artificial Sequence
  B2M-targeting guide sequence portion
cuucaaugucggauggaugaa

SEQ ID NO: 800 - RNA, 21 nt, Artificial Sequence
  B2M-targeting guide sequence portion
cuucaguaagucaacuucaau

SEQ ID NO: 801 - RNA, 21 nt, Artificial Sequence
  B2M-targeting guide sequence portion
cuugggcugugacaaagucac

SEQ ID NO: 802 - RNA, 21 nt, Artificial Sequence
  B2M-targeting guide sequence portion
cuuguacuacacugaauucac

SEQ ID NO: 803 - RNA, 21 nt, Artificial Sequence
  B2M-targeting guide sequence portion
cuuuccauucucugcuggaug

SEQ ID NO: 804 - RNA, 21 nt, Artificial Sequence
  B2M-targeting guide sequence portion
cuuucuaucucuuguacuaca

SEQ ID NO: 805 - RNA, 21 nt, Artificial Sequence
  B2M-targeting guide sequence portion
cuuuuucagugggggugaauu

SEQ ID NO: 806 - RNA, 21 nt, Artificial Sequence
  B2M-targeting guide sequence portion
gaaaaagaugaguaugccugc

SEQ ID NO: 807 - RNA, 21 nt, Artificial Sequence
  B2M-targeting guide sequence portion
gaaaaaguggagcauucagac

SEQ ID NO: 808 - RNA, 21 nt, Artificial Sequence
  B2M-targeting guide sequence portion
gaaacccagacacauagcaau

SEQ ID NO: 809 - RNA, 21 nt, Artificial Sequence
  B2M-targeting guide sequence portion
gaaagacaagucugaaugcuc

SEQ ID NO: 810 - RNA, 21 nt, Artificial Sequence
  B2M-targeting guide sequence portion
gaaagaccaguccuugcugaa

SEQ ID NO: 811 - RNA, 21 nt, Artificial Sequence
  B2M-targeting guide sequence portion
gaaagucaaauuuccugaauu

SEQ ID NO: 812 - RNA, 21 nt, Artificial Sequence
  B2M-targeting guide sequence portion
gaaauuugacuuuccauucuc

SEQ ID NO: 813 - RNA, 21 nt, Artificial Sequence
  B2M-targeting guide sequence portion
gaaccaugugacuuugucaca

SEQ ID NO: 814 - RNA, 21 nt, Artificial Sequence
  B2M-targeting guide sequence portion
gaagaauggagagagaauuga

SEQ ID NO: 815 - RNA, 21 nt, Artificial Sequence
  B2M-targeting guide sequence portion
gaaguugacuuacugaagaau

SEQ ID NO: 816 - RNA, 21 nt, Artificial Sequence
  B2M-targeting guide sequence portion
gaaugcuccacuuuuucaauu

SEQ ID NO: 817 - RNA, 21 nt, Artificial Sequence
  B2M-targeting guide sequence portion
gaauggaaagucaaauuuccu

SEQ ID NO: 818 - RNA, 21 nt, Artificial Sequence
  B2M-targeting guide sequence portion
gaauggagagagaauugaaaa

SEQ ID NO: 819 - RNA, 21 nt, Artificial Sequence
  B2M-targeting guide sequence portion
gaauucacccccacugaaaaa

SEQ ID NO: 820 - RNA, 21 nt, Artificial Sequence
  B2M-targeting guide sequence portion
gaauucaguguaguacaagag

SEQ ID NO: 821 - RNA, 21 nt, Artificial Sequence
  B2M-targeting guide sequence portion
gaauugaaaaaguggagcauu

SEQ ID NO: 822 - RNA, 21 nt, Artificial Sequence
  B2M-targeting guide sequence portion
gaauugcuaugugucuggguu

SEQ ID NO: 823 - RNA, 21 nt, Artificial Sequence
  B2M-targeting guide sequence portion
gacaagucugaaugcuccacu

SEQ ID NO: 824 - RNA, 21 nt, Artificial Sequence
  B2M-targeting guide sequence portion
gacacauagcaauucaggaaa

SEQ ID NO: 825 - RNA, 21 nt, Artificial Sequence
  B2M-targeting guide sequence portion
gacauugaaguugacuuacug

SEQ ID NO: 826 - RNA, 21 nt, Artificial Sequence
  B2M-targeting guide sequence portion
gaccaguccuugcugaaagac

SEQ ID NO: 827 - RNA, 21 nt, Artificial Sequence
  B2M-targeting guide sequence portion
gacgugaguaaaccugaaucu

SEQ ID NO: 828 - RNA, 21 nt, Artificial Sequence
  B2M-targeting guide sequence portion
gacuggucuuucuaucucuug

SEQ ID NO: 829 - RNA, 21 nt, Artificial Sequence
  B2M-targeting guide sequence portion
gacuuaccccacuuaacuauc

SEQ ID NO: 830 - RNA, 21 nt, Artificial Sequence
  B2M-targeting guide sequence portion
gacuuacugaagaauggagag

SEQ ID NO: 831 - RNA, 21 nt, Artificial Sequence
  B2M-targeting guide sequence portion
gacuuuccauucucugcugga

SEQ ID NO: 832 - RNA, 21 nt, Artificial Sequence
  B2M-targeting guide sequence portion
gacuuugucacagcccaagau

SEQ ID NO: 833 - RNA, 21 nt, Artificial Sequence
  B2M-targeting guide sequence portion
gagaauggaaagucaaauuuc

SEQ ID NO: 834 - RNA, 21 nt, Artificial Sequence
  B2M-targeting guide sequence portion
gagaauugaaaaaguggagca

SEQ ID NO: 835 - RNA, 21 nt, Artificial Sequence
  B2M-targeting guide sequence portion
gagagaauugaaaaaguggag

SEQ ID NO: 836 - RNA, 21 nt, Artificial Sequence
  B2M-targeting guide sequence portion
gagagagaauugaaaaagugg

SEQ ID NO: 837 - RNA, 21 nt, Artificial Sequence
  B2M-targeting guide sequence portion
gagauagaaagaccaguccuu

SEQ ID NO: 838 - RNA, 21 nt, Artificial Sequence
  B2M-targeting guide sequence portion
gagcauucagacuugucuuuc

SEQ ID NO: 839 - RNA, 21 nt, Artificial Sequence
  B2M-targeting guide sequence portion
gaguaaaccugaaucuuugga

SEQ ID NO: 840 - RNA, 21 nt, Artificial Sequence
  B2M-targeting guide sequence portion
gaguaugccugccgugugaac

SEQ ID NO: 841 - RNA, 21 nt, Artificial Sequence
  B2M-targeting guide sequence portion
gauagaaagaccaguccuugc

SEQ ID NO: 842 - RNA, 21 nt, Artificial Sequence
  B2M-targeting guide sequence portion
gaugaaacccagacacauagc

SEQ ID NO: 843 - RNA, 21 nt, Artificial Sequence
  B2M-targeting guide sequence portion
gaugacgugaguaaaccugaa

SEQ ID NO: 844 - RNA, 21 nt, Artificial Sequence
  B2M-targeting guide sequence portion
gauggaugaaacccagacaca

SEQ ID NO: 845 - RNA, 21 nt, Artificial Sequence
  B2M-targeting guide sequence portion
gauucagguuuacucacguca

SEQ ID NO: 846 - RNA, 21 nt, Artificial Sequence
  B2M-targeting guide sequence portion
gcaauucaggaaauuugacuu

SEQ ID NO: 847 - RNA, 21 nt, Artificial Sequence
  B2M-targeting guide sequence portion
gcagagaauggaaagucaaau

SEQ ID NO: 848 - RNA, 21 nt, Artificial Sequence
  B2M-targeting guide sequence portion
gcaggcauacucaucuuuuuc

SEQ ID NO: 849 - RNA, 21 nt, Artificial Sequence
  B2M-targeting guide sequence portion
gcauucagacuugucuuucag

SEQ ID NO: 850 - RNA, 21 nt, Artificial Sequence
  B2M-targeting guide sequence portion
gcccaagauaguuaagugggg

SEQ ID NO: 851 - RNA, 21 nt, Artificial Sequence
  B2M-targeting guide sequence portion
gccgugugaaccaugugacuu

SEQ ID NO: 852 - RNA, 21 nt, Artificial Sequence
  B2M-targeting guide sequence portion
gccugccgugugaaccaugug

SEQ ID NO: 853 - RNA, 21 nt, Artificial Sequence
  B2M-targeting guide sequence portion
gcuaugugucuggguuucauc

SEQ ID NO: 854 - RNA, 21 nt, Artificial Sequence
  B2M-targeting guide sequence portion
gcuccacuuuuucaauucucu

SEQ ID NO: 855 - RNA, 21 nt, Artificial Sequence
  B2M-targeting guide sequence portion
gcugaaagacaagucugaaug

SEQ ID NO: 856 - RNA, 21 nt, Artificial Sequence
  B2M-targeting guide sequence portion
gcugugacaaagucacauggu

SEQ ID NO: 857 - RNA, 21 nt, Artificial Sequence
  B2M-targeting guide sequence portion
ggaaagucaaauuuccugaau

SEQ ID NO: 858 - RNA, 21 nt, Artificial Sequence
  B2M-targeting guide sequence portion
ggaaauuugacuuuccauucu

SEQ ID NO: 859 - RNA, 21 nt, Artificial Sequence
  B2M-targeting guide sequence portion
ggacuggucuuucuaucucuu

SEQ ID NO: 860 - RNA, 21 nt, Artificial Sequence
  B2M-targeting guide sequence portion
ggagagagaauugaaaaagug

SEQ ID NO: 861 - RNA, 21 nt, Artificial Sequence
  B2M-targeting guide sequence portion
ggagcauucagacuugucuuu

SEQ ID NO: 862 - RNA, 21 nt, Artificial Sequence
  B2M-targeting guide sequence portion
ggaugaaacccagacacauag

SEQ ID NO: 863 - RNA, 21 nt, Artificial Sequence
  B2M-targeting guide sequence portion
ggaugacgugaguaaaccuga

SEQ ID NO: 864 - RNA, 21 nt, Artificial Sequence
  B2M-targeting guide sequence portion
ggauggaugaaacccagacac

SEQ ID NO: 865 - RNA, 21 nt, Artificial Sequence
  B2M-targeting guide sequence portion
ggcaggcauacucaucuuuuu

SEQ ID NO: 866 - RNA, 21 nt, Artificial Sequence
  B2M-targeting guide sequence portion
ggcauacucaucuuuuucagu

SEQ ID NO: 867 - RNA, 21 nt, Artificial Sequence
  B2M-targeting guide sequence portion
gggcugugacaaagucacaug

SEQ ID NO: 868 - RNA, 21 nt, Artificial Sequence
  B2M-targeting guide sequence portion
gggggugaauucaguguagua

SEQ ID NO: 869 - RNA, 21 nt, Artificial Sequence
  B2M-targeting guide sequence portion
ggggugaauucaguguaguac

SEQ ID NO: 870 - RNA, 21 nt, Artificial Sequence
  B2M-targeting guide sequence portion
gggugaauucaguguaguaca

SEQ ID NO: 871 - RNA, 21 nt, Artificial Sequence
  B2M-targeting guide sequence portion
ggguuucauccauccgacauu

SEQ ID NO: 872 - RNA, 21 nt, Artificial Sequence
  B2M-targeting guide sequence portion
gguacuccaaagauucagguu

SEQ ID NO: 873 - RNA, 21 nt, Artificial Sequence
  B2M-targeting guide sequence portion
ggucuuucuaucucuuguacu

SEQ ID NO: 874 - RNA, 21 nt, Artificial Sequence
  B2M-targeting guide sequence portion
ggugaauucaguguaguacaa

SEQ ID NO: 875 - RNA, 21 nt, Artificial Sequence
  B2M-targeting guide sequence portion
gguucacacggcaggcauacu

SEQ ID NO: 876 - RNA, 21 nt, Artificial Sequence
  B2M-targeting guide sequence portion
gguuuacucacgucauccagc

SEQ ID NO: 877 - RNA, 21 nt, Artificial Sequence
  B2M-targeting guide sequence portion
gguuucauccauccgacauug

SEQ ID NO: 878 - RNA, 21 nt, Artificial Sequence
  B2M-targeting guide sequence portion
guaaaccugaaucuuuggagu

SEQ ID NO: 879 - RNA, 21 nt, Artificial Sequence
  B2M-targeting guide sequence portion
guaagucaacuucaaugucgg

SEQ ID NO: 880 - RNA, 21 nt, Artificial Sequence
  B2M-targeting guide sequence portion
guacaagagauagaaagacca

SEQ ID NO: 881 - RNA, 21 nt, Artificial Sequence
  B2M-targeting guide sequence portion
guacuacacugaauucacccc

SEQ ID NO: 882 - RNA, 21 nt, Artificial Sequence
  B2M-targeting guide sequence portion
guaguacaagagauagaaaga

SEQ ID NO: 883 - RNA, 21 nt, Artificial Sequence
  B2M-targeting guide sequence portion
gucaaauuuccugaauugcua

SEQ ID NO: 884 - RNA, 21 nt, Artificial Sequence
  B2M-targeting guide sequence portion
gucacagcccaagauaguuaa

SEQ ID NO: 885 - RNA, 21 nt, Artificial Sequence
  B2M-targeting guide sequence portion
gucauccagcagagaauggaa

SEQ ID NO: 886 - RNA, 21 nt, Artificial Sequence
  B2M-targeting guide sequence portion
guccuugcugaaagacaaguc

SEQ ID NO: 887 - RNA, 21 nt, Artificial Sequence
  B2M-targeting guide sequence portion
gucggauggaugaaacccaga

SEQ ID NO: 888 - RNA, 21 nt, Artificial Sequence
  B2M-targeting guide sequence portion
gucuuucagcaaggacugguc

SEQ ID NO: 889 - RNA, 21 nt, Artificial Sequence
  B2M-targeting guide sequence portion
gucuuucuaucucuuguacua

SEQ ID NO: 890 - RNA, 21 nt, Artificial Sequence
  B2M-targeting guide sequence portion
gugaaccaugugacuuuguca

SEQ ID NO: 891 - RNA, 21 nt, Artificial Sequence
  B2M-targeting guide sequence portion
gugacaaagucacaugguuca

SEQ ID NO: 892 - RNA, 21 nt, Artificial Sequence
  B2M-targeting guide sequence portion
gugacuuugucacagcccaag

SEQ ID NO: 893 - RNA, 21 nt, Artificial Sequence
  B2M-targeting guide sequence portion
guggagcauucagacuugucu

SEQ ID NO: 894 - RNA, 21 nt, Artificial Sequence
  B2M-targeting guide sequence portion
gugggggugaauucaguguag

SEQ ID NO: 895 - RNA, 21 nt, Artificial Sequence
  B2M-targeting guide sequence portion
gugucuggguuucauccaucc

SEQ ID NO: 896 - RNA, 21 nt, Artificial Sequence
  B2M-targeting guide sequence portion
guucacacggcaggcauacuc

SEQ ID NO: 897 - RNA, 21 nt, Artificial Sequence
  B2M-targeting guide sequence portion
guuuacucacgucauccagca

SEQ ID NO: 898 - RNA, 21 nt, Artificial Sequence
  B2M-targeting guide sequence portion
guuucauccauccgacauuga

SEQ ID NO: 899 - RNA, 21 nt, Artificial Sequence
  B2M-targeting guide sequence portion
uaaaccugaaucuuuggagua

SEQ ID NO: 900 - RNA, 21 nt, Artificial Sequence
  B2M-targeting guide sequence portion
uaacuaucuugggcugugaca

SEQ ID NO: 901 - RNA, 21 nt, Artificial Sequence
  B2M-targeting guide sequence portion
uaagucaacuucaaugucgga

SEQ ID NO: 902 - RNA, 21 nt, Artificial Sequence
  B2M-targeting guide sequence portion
uacaagagauagaaagaccag

SEQ ID NO: 903 - RNA, 21 nt, Artificial Sequence
  B2M-targeting guide sequence portion
uacacugaauucacccccacu

SEQ ID NO: 904 - RNA, 21 nt, Artificial Sequence
  B2M-targeting guide sequence portion
uaccccacuuaacuaucuugg

SEQ ID NO: 905 - RNA, 21 nt, Artificial Sequence
  B2M-targeting guide sequence portion
uacuacacugaauucaccccc

SEQ ID NO: 906 - RNA, 21 nt, Artificial Sequence
  B2M-targeting guide sequence portion
uacucacgucauccagcagag

SEQ ID NO: 907 - RNA, 21 nt, Artificial Sequence
  B2M-targeting guide sequence portion
uacucaucuuuuucagugggg

SEQ ID NO: 908 - RNA, 21 nt, Artificial Sequence
  B2M-targeting guide sequence portion
uacuccaaagauucagguuua

SEQ ID NO: 909 - RNA, 21 nt, Artificial Sequence
  B2M-targeting guide sequence portion
uacugaagaauggagagagaa

SEQ ID NO: 910 - RNA, 21 nt, Artificial Sequence
  B2M-targeting guide sequence portion
uagcaauucaggaaauuugac

SEQ ID NO: 911 - RNA, 21 nt, Artificial Sequence
  B2M-targeting guide sequence portion
uaguacaagagauagaaagac

SEQ ID NO: 912 - RNA, 21 nt, Artificial Sequence
  B2M-targeting guide sequence portion
uaugugucuggguuucaucca

SEQ ID NO: 913 - RNA, 21 nt, Artificial Sequence
  B2M-targeting guide sequence portion
ucaaauuuccugaauugcuau

SEQ ID NO: 914 - RNA, 21 nt, Artificial Sequence
  B2M-targeting guide sequence portion
ucaacuucaaugucggaugga

SEQ ID NO: 915 - RNA, 21 nt, Artificial Sequence
  B2M-targeting guide sequence portion
ucaaugucggauggaugaaac

SEQ ID NO: 916 - RNA, 21 nt, Artificial Sequence
  B2M-targeting guide sequence portion
ucacacggcaggcauacucau

SEQ ID NO: 917 - RNA, 21 nt, Artificial Sequence
  B2M-targeting guide sequence portion
ucacccccacugaaaaagaug

SEQ ID NO: 918 - RNA, 21 nt, Artificial Sequence
  B2M-targeting guide sequence portion
ucacgucauccagcagagaau

SEQ ID NO: 919 - RNA, 21 nt, Artificial Sequence
  B2M-targeting guide sequence portion
ucagacuugucuuucagcaag

SEQ ID NO: 920 - RNA, 21 nt, Artificial Sequence
  B2M-targeting guide sequence portion
ucagcaaggacuggucuuucu

SEQ ID NO: 921 - RNA, 21 nt, Artificial Sequence
  B2M-targeting guide sequence portion
ucaggaaauuugacuuuccau

SEQ ID NO: 922 - RNA, 21 nt, Artificial Sequence
  B2M-targeting guide sequence portion
ucagguacuccaaagauucag

SEQ ID NO: 923 - RNA, 21 nt, Artificial Sequence
  B2M-targeting guide sequence portion
ucaguaagucaacuucaaugu

SEQ ID NO: 924 - RNA, 21 nt, Artificial Sequence
  B2M-targeting guide sequence portion
ucaguguaguacaagagauag

SEQ ID NO: 925 - RNA, 21 nt, Artificial Sequence
  B2M-targeting guide sequence portion
ucauccagcagagaauggaaa

SEQ ID NO: 926 - RNA, 21 nt, Artificial Sequence
  B2M-targeting guide sequence portion
ucauccauccgacauugaagu

SEQ ID NO: 927 - RNA, 21 nt, Artificial Sequence
  B2M-targeting guide sequence portion
ucaucuuuuucagugggggug

SEQ ID NO: 928 - RNA, 21 nt, Artificial Sequence
  B2M-targeting guide sequence portion
uccaaagauucagguuuacuc

SEQ ID NO: 929 - RNA, 21 nt, Artificial Sequence
  B2M-targeting guide sequence portion
uccacuuuuucaauucucucu

SEQ ID NO: 930 - RNA, 21 nt, Artificial Sequence
  B2M-targeting guide sequence portion
uccagcagagaauggaaaguc

SEQ ID NO: 931 - RNA, 21 nt, Artificial Sequence
  B2M-targeting guide sequence portion
uccauccgacauugaaguuga

SEQ ID NO: 932 - RNA, 21 nt, Artificial Sequence
  B2M-targeting guide sequence portion
uccauucuucaguaagucaac

SEQ ID NO: 933 - RNA, 21 nt, Artificial Sequence
  B2M-targeting guide sequence portion
uccucagguacuccaaagauu

SEQ ID NO: 934 - RNA, 21 nt, Artificial Sequence
  B2M-targeting guide sequence portion
uccugaauugcuaugugucug

SEQ ID NO: 935 - RNA, 21 nt, Artificial Sequence
  B2M-targeting guide sequence portion
uccuugcugaaagacaagucu

SEQ ID NO: 936 - RNA, 21 nt, Artificial Sequence
  B2M-targeting guide sequence portion
ucggauggaugaaacccagac

SEQ ID NO: 937 - RNA, 21 nt, Artificial Sequence
  B2M-targeting guide sequence portion
ucuaucucuuguacuacacug

SEQ ID NO: 938 - RNA, 21 nt, Artificial Sequence
  B2M-targeting guide sequence portion
ucuccauucuucaguaaguca

SEQ ID NO: 939 - RNA, 21 nt, Artificial Sequence
  B2M-targeting guide sequence portion
ucucuccauucuucaguaagu

SEQ ID NO: 940 - RNA, 21 nt, Artificial Sequence
  B2M-targeting guide sequence portion
ucucucuccauucuucaguaa

SEQ ID NO: 941 - RNA, 21 nt, Artificial Sequence
  B2M-targeting guide sequence portion
ucucugcuggaugacgugagu

SEQ ID NO: 942 - RNA, 21 nt, Artificial Sequence
  B2M-targeting guide sequence portion
ucucuuguacuacacugaauu

SEQ ID NO: 943 - RNA, 21 nt, Artificial Sequence
  B2M-targeting guide sequence portion
ucugaaugcuccacuuuuuca

SEQ ID NO: 944 - RNA, 21 nt, Artificial Sequence
  B2M-targeting guide sequence portion
ucugcuggaugacgugaguaa

SEQ ID NO: 945 - RNA, 21 nt, Artificial Sequence
  B2M-targeting guide sequence portion
ucuggguuucauccauccgac

SEQ ID NO: 946 - RNA, 21 nt, Artificial Sequence
  B2M-targeting guide sequence portion
ucuucaguaagucaacuucaa

SEQ ID NO: 947 - RNA, 21 nt, Artificial Sequence
  B2M-targeting guide sequence portion
ucuugggcugugacaaaguca

SEQ ID NO: 948 - RNA, 21 nt, Artificial Sequence
  B2M-targeting guide sequence portion
ucuuguacuacacugaauuca

SEQ ID NO: 949 - RNA, 21 nt, Artificial Sequence
  B2M-targeting guide sequence portion
ucuuucagcaaggacuggucu

SEQ ID NO: 950 - RNA, 21 nt, Artificial Sequence
  B2M-targeting guide sequence portion
ucuuucuaucucuuguacuac

SEQ ID NO: 951 - RNA, 21 nt, Artificial Sequence
  B2M-targeting guide sequence portion
ucuuuuucagugggggugaau

SEQ ID NO: 952 - RNA, 21 nt, Artificial Sequence
  B2M-targeting guide sequence portion
ugaaaaagaugaguaugccug

SEQ ID NO: 953 - RNA, 21 nt, Artificial Sequence
  B2M-targeting guide sequence portion
ugaaaaaguggagcauucaga

SEQ ID NO: 954 - RNA, 21 nt, Artificial Sequence
  B2M-targeting guide sequence portion
ugaaacccagacacauagcaa

SEQ ID NO: 955 - RNA, 21 nt, Artificial Sequence
  B2M-targeting guide sequence portion
ugaaagacaagucugaaugcu

SEQ ID NO: 956 - RNA, 21 nt, Artificial Sequence
  B2M-targeting guide sequence portion
ugaaccaugugacuuugucac

SEQ ID NO: 957 - RNA, 21 nt, Artificial Sequence
  B2M-targeting guide sequence portion
ugaagaauggagagagaauug

SEQ ID NO: 958 - RNA, 21 nt, Artificial Sequence
  B2M-targeting guide sequence portion
ugaaguugacuuacugaagaa

SEQ ID NO: 959 - RNA, 21 nt, Artificial Sequence
  B2M-targeting guide sequence portion
ugaaugcuccacuuuuucaau

SEQ ID NO: 960 - RNA, 21 nt, Artificial Sequence
  B2M-targeting guide sequence portion
ugaauucacccccacugaaaa

SEQ ID NO: 961 - RNA, 21 nt, Artificial Sequence
  B2M-targeting guide sequence portion
ugaauucaguguaguacaaga

SEQ ID NO: 962 - RNA, 21 nt, Artificial Sequence
  B2M-targeting guide sequence portion
ugacaaagucacaugguucac

SEQ ID NO: 963 - RNA, 21 nt, Artificial Sequence
  B2M-targeting guide sequence portion
ugacgugaguaaaccugaauc

SEQ ID NO: 964 - RNA, 21 nt, Artificial Sequence
  B2M-targeting guide sequence portion
ugacuuacugaagaauggaga

SEQ ID NO: 965 - RNA, 21 nt, Artificial Sequence
  B2M-targeting guide sequence portion
ugacuuuccauucucugcugg

SEQ ID NO: 966 - RNA, 21 nt, Artificial Sequence
  B2M-targeting guide sequence portion
ugacuuugucacagcccaaga

SEQ ID NO: 967 - RNA, 21 nt, Artificial Sequence
  B2M-targeting guide sequence portion
ugaguaaaccugaaucuuugg

SEQ ID NO: 968 - RNA, 21 nt, Artificial Sequence
  B2M-targeting guide sequence portion
ugaguaugccugccgugugaa

SEQ ID NO: 969 - RNA, 21 nt, Artificial Sequence
  B2M-targeting guide sequence portion
ugccgugugaaccaugugacu

SEQ ID NO: 970 - RNA, 21 nt, Artificial Sequence
  B2M-targeting guide sequence portion
ugcuaugugucuggguuucau

SEQ ID NO: 971 - RNA, 21 nt, Artificial Sequence
  B2M-targeting guide sequence portion
ugcuccacuuuuucaauucuc

SEQ ID NO: 972 - RNA, 21 nt, Artificial Sequence
  B2M-targeting guide sequence portion
ugcugaaagacaagucugaau

SEQ ID NO: 973 - RNA, 21 nt, Artificial Sequence
  B2M-targeting guide sequence portion
ugcuggaugacgugaguaaac

SEQ ID NO: 974 - RNA, 21 nt, Artificial Sequence
  B2M-targeting guide sequence portion
uggaaagucaaauuuccugaa

SEQ ID NO: 975 - RNA, 21 nt, Artificial Sequence
  B2M-targeting guide sequence portion
uggagagagaauugaaaaagu

SEQ ID NO: 976 - RNA, 21 nt, Artificial Sequence
  B2M-targeting guide sequence portion
uggagcauucagacuugucuu

SEQ ID NO: 977 - RNA, 21 nt, Artificial Sequence
  B2M-targeting guide sequence portion
uggaugaaacccagacacaua

SEQ ID NO: 978 - RNA, 21 nt, Artificial Sequence
  B2M-targeting guide sequence portion
ugggcugugacaaagucacau

SEQ ID NO: 979 - RNA, 21 nt, Artificial Sequence
  B2M-targeting guide sequence portion
ugggggugaauucaguguagu

SEQ ID NO: 980 - RNA, 21 nt, Artificial Sequence
  B2M-targeting guide sequence portion
uggguuucauccauccgacau

SEQ ID NO: 981 - RNA, 21 nt, Artificial Sequence
  B2M-targeting guide sequence portion
uggucuuucuaucucuuguac

SEQ ID NO: 982 - RNA, 21 nt, Artificial Sequence
  B2M-targeting guide sequence portion
ugguucacacggcaggcauac

SEQ ID NO: 983 - RNA, 21 nt, Artificial Sequence
  B2M-targeting guide sequence portion
uguacuacacugaauucaccc

SEQ ID NO: 984 - RNA, 21 nt, Artificial Sequence
  B2M-targeting guide sequence portion
uguaguacaagagauagaaag

SEQ ID NO: 985 - RNA, 21 nt, Artificial Sequence
  B2M-targeting guide sequence portion
ugucacagcccaagauaguua

SEQ ID NO: 986 - RNA, 21 nt, Artificial Sequence
  B2M-targeting guide sequence portion
ugucggauggaugaaacccag

SEQ ID NO: 987 - RNA, 21 nt, Artificial Sequence
  B2M-targeting guide sequence portion
ugucuggguuucauccauccg

SEQ ID NO: 988 - RNA, 21 nt, Artificial Sequence
  B2M-targeting guide sequence portion
ugucuuucagcaaggacuggu

SEQ ID NO: 989 - RNA, 21 nt, Artificial Sequence
  B2M-targeting guide sequence portion
ugugaaccaugugacuuuguc

SEQ ID NO: 990 - RNA, 21 nt, Artificial Sequence
  B2M-targeting guide sequence portion
ugugacaaagucacaugguuc

SEQ ID NO: 991 - RNA, 21 nt, Artificial Sequence
  B2M-targeting guide sequence portion
ugugacuuugucacagcccaa

SEQ ID NO: 992 - RNA, 21 nt, Artificial Sequence
  B2M-targeting guide sequence portion
ugugucuggguuucauccauc

SEQ ID NO: 993 - RNA, 21 nt, Artificial Sequence
  B2M-targeting guide sequence portion
uuacucacgucauccagcaga

SEQ ID NO: 994 - RNA, 21 nt, Artificial Sequence
  B2M-targeting guide sequence portion
uuacugaagaauggagagaga

SEQ ID NO: 995 - RNA, 21 nt, Artificial Sequence
  B2M-targeting guide sequence portion
uucaaugucggauggaugaaa

SEQ ID NO: 996 - RNA, 21 nt, Artificial Sequence
  B2M-targeting guide sequence portion
uucacccccacugaaaaagau

SEQ ID NO: 997 - RNA, 21 nt, Artificial Sequence
  B2M-targeting guide sequence portion
uucagcaaggacuggucuuuc

SEQ ID NO: 998 - RNA, 21 nt, Artificial Sequence
  B2M-targeting guide sequence portion
uucaggaaauuugacuuucca

SEQ ID NO: 999 - RNA, 21 nt, Artificial Sequence
  B2M-targeting guide sequence portion
uucagguuuacucacgucauc

SEQ ID NO: 1000 - RNA, 21 nt, Artificial Sequence
  B2M-targeting guide sequence portion
uucaguaagucaacuucaaug

SEQ ID NO: 1001 - RNA, 21 nt, Artificial Sequence
  B2M-targeting guide sequence portion
uucagugggggugaauucagu

SEQ ID NO: 1002 - RNA, 21 nt, Artificial Sequence
  B2M-targeting guide sequence portion
uucaguguaguacaagagaua

SEQ ID NO: 1003 - RNA, 21 nt, Artificial Sequence
  B2M-targeting guide sequence portion
uucauccauccgacauugaag

SEQ ID NO: 1004 - RNA, 21 nt, Artificial Sequence
  B2M-targeting guide sequence portion
uuccauucucugcuggaugac

SEQ ID NO: 1005 - RNA, 21 nt, Artificial Sequence
  B2M-targeting guide sequence portion
uucuaucucuuguacuacacu

SEQ ID NO: 1006 - RNA, 21 nt, Artificial Sequence
  B2M-targeting guide sequence portion
uucucugcuggaugacgugag

SEQ ID NO: 1007 - RNA, 21 nt, Artificial Sequence
  B2M-targeting guide sequence portion
uugaaaaaguggagcauucag

SEQ ID NO: 1008 - RNA, 21 nt, Artificial Sequence
  B2M-targeting guide sequence portion
uugacuuacugaagaauggag

SEQ ID NO: 1009 - RNA, 21 nt, Artificial Sequence
  B2M-targeting guide sequence portion
uugcuaugugucuggguuuca

SEQ ID NO: 1010 - RNA, 21 nt, Artificial Sequence
  B2M-targeting guide sequence portion
uugcugaaagacaagucugaa

SEQ ID NO: 1011 - RNA, 21 nt, Artificial Sequence
  B2M-targeting guide sequence portion
uugggcugugacaaagucaca

SEQ ID NO: 1012 - RNA, 21 nt, Artificial Sequence
  B2M-targeting guide sequence portion
uuguacuacacugaauucacc

SEQ ID NO: 1013 - RNA, 21 nt, Artificial Sequence
  B2M-targeting guide sequence portion
uugucacagcccaagauaguu

SEQ ID NO: 1014 - RNA, 21 nt, Artificial Sequence
  B2M-targeting guide sequence portion
uugucuuucagcaaggacugg

SEQ ID NO: 1015 - RNA, 21 nt, Artificial Sequence
  B2M-targeting guide sequence portion
uuuacucacgucauccagcag

SEQ ID NO: 1016 - RNA, 21 nt, Artificial Sequence
  B2M-targeting guide sequence portion
uuucaauucucucuccauucu

SEQ ID NO: 1017 - RNA, 21 nt, Artificial Sequence
  B2M-targeting guide sequence portion
uuucagcaaggacuggucuuu

SEQ ID NO: 1018 - RNA, 21 nt, Artificial Sequence
  B2M-targeting guide sequence portion
uuucagugggggugaauucag

SEQ ID NO: 1019 - RNA, 21 nt, Artificial Sequence
  B2M-targeting guide sequence portion
uuuccugaauugcuauguguc

SEQ ID NO: 1020 - RNA, 21 nt, Artificial Sequence
  B2M-targeting guide sequence portion
uuugacuuuccauucucugcu

SEQ ID NO: 1021 - RNA, 21 nt, Artificial Sequence
  B2M-targeting guide sequence portion
uuugucacagcccaagauagu

SEQ ID NO: 1022 - RNA, 21 nt, Artificial Sequence
  B2M-targeting guide sequence portion
uuuucaauucucucuccauuc

SEQ ID NO: 1023 - RNA, 21 nt, Artificial Sequence
  B2M-targeting guide sequence portion
uuuucagugggggugaauuca

SEQ ID NO: 1024 - RNA, 21 nt, Artificial Sequence
  B2M-targeting guide sequence portion
uuuuucaauucucucuccauu

SEQ ID NO: 1025 - RNA, 22 nt, Artificial Sequence
  B2M-targeting guide sequence portion
aaaaagaugaguaugccugccg

SEQ ID NO: 1026 - RNA, 22 nt, Artificial Sequence
  B2M-targeting guide sequence portion
aaaaaguggagcauucagacuu

SEQ ID NO: 1027 - RNA, 22 nt, Artificial Sequence
  B2M-targeting guide sequence portion
aaaagaugaguaugccugccgu

SEQ ID NO: 1028 - RNA, 22 nt, Artificial Sequence
  B2M-targeting guide sequence portion
aaaaguggagcauucagacuug

SEQ ID NO: 1029 - RNA, 22 nt, Artificial Sequence
  B2M-targeting guide sequence portion
aaacccagacacauagcaauuc

SEQ ID NO: 1030 - RNA, 22 nt, Artificial Sequence
  B2M-targeting guide sequence portion
aaaccugaaucuuuggaguacc

SEQ ID NO: 1031 - RNA, 22 nt, Artificial Sequence
  B2M-targeting guide sequence portion
aaagacaagucugaaugcucca

SEQ ID NO: 1032 - RNA, 22 nt, Artificial Sequence
  B2M-targeting guide sequence portion
aaagaccaguccuugcugaaag

SEQ ID NO: 1033 - RNA, 22 nt, Artificial Sequence
  B2M-targeting guide sequence portion
aaagaugaguaugccugccgug

SEQ ID NO: 1034 - RNA, 22 nt, Artificial Sequence
  B2M-targeting guide sequence portion
aaagauucagguuuacucacgu

SEQ ID NO: 1035 - RNA, 22 nt, Artificial Sequence
  B2M-targeting guide sequence portion
aaagucaaauuuccugaauugc

SEQ ID NO: 1036 - RNA, 22 nt, Artificial Sequence
  B2M-targeting guide sequence portion
aaagucacaugguucacacggc

SEQ ID NO: 1037 - RNA, 22 nt, Artificial Sequence
  B2M-targeting guide sequence portion
aaaguggagcauucagacuugu

SEQ ID NO: 1038 - RNA, 22 nt, Artificial Sequence
  B2M-targeting guide sequence portion
aaauuuccugaauugcuaugug

SEQ ID NO: 1039 - RNA, 22 nt, Artificial Sequence
  B2M-targeting guide sequence portion
aaauuugacuuuccauucucug

SEQ ID NO: 1040 - RNA, 22 nt, Artificial Sequence
  B2M-targeting guide sequence portion
aaccaugugacuuugucacagc

SEQ ID NO: 1041 - RNA, 22 nt, Artificial Sequence
  B2M-targeting guide sequence portion
aacccagacacauagcaauuca

SEQ ID NO: 1042 - RNA, 22 nt, Artificial Sequence
  B2M-targeting guide sequence portion
aaccugaaucuuuggaguaccu

SEQ ID NO: 1043 - RNA, 22 nt, Artificial Sequence
  B2M-targeting guide sequence portion
aacuaucuugggcugugacaaa

SEQ ID NO: 1044 - RNA, 22 nt, Artificial Sequence
  B2M-targeting guide sequence portion
aacuucaaugucggauggauga

SEQ ID NO: 1045 - RNA, 22 nt, Artificial Sequence
  B2M-targeting guide sequence portion
aagaauggagagagaauugaaa

SEQ ID NO: 1046 - RNA, 22 nt, Artificial Sequence
  B2M-targeting guide sequence portion
aagaccaguccuugcugaaaga

SEQ ID NO: 1047 - RNA, 22 nt, Artificial Sequence
  B2M-targeting guide sequence portion
aagagauagaaagaccaguccu

SEQ ID NO: 1048 - RNA, 22 nt, Artificial Sequence
  B2M-targeting guide sequence portion
aagaugaguaugccugccgugu

SEQ ID NO: 1049 - RNA, 22 nt, Artificial Sequence
  B2M-targeting guide sequence portion
aagauucagguuuacucacguc

SEQ ID NO: 1050 - RNA, 22 nt, Artificial Sequence
  B2M-targeting guide sequence portion
aaggacuggucuuucuaucucu

SEQ ID NO: 1051 - RNA, 22 nt, Artificial Sequence
  B2M-targeting guide sequence portion
aagucaaauuuccugaauugcu

SEQ ID NO: 1052 - RNA, 22 nt, Artificial Sequence
  B2M-targeting guide sequence portion
aagucaacuucaaugucggaug

SEQ ID NO: 1053 - RNA, 22 nt, Artificial Sequence
  B2M-targeting guide sequence portion
aagucacaugguucacacggca

SEQ ID NO: 1054 - RNA, 22 nt, Artificial Sequence
  B2M-targeting guide sequence portion
aagucugaaugcuccacuuuuu

SEQ ID NO: 1055 - RNA, 22 nt, Artificial Sequence
  B2M-targeting guide sequence portion
aaguggagcauucagacuuguc

SEQ ID NO: 1056 - RNA, 22 nt, Artificial Sequence
  B2M-targeting guide sequence portion
aaguugacuuacugaagaaugg

SEQ ID NO: 1057 - RNA, 22 nt, Artificial Sequence
  B2M-targeting guide sequence portion
aaugcuccacuuuuucaauucu

SEQ ID NO: 1058 - RNA, 22 nt, Artificial Sequence
  B2M-targeting guide sequence portion
aauggaaagucaaauuuccuga

SEQ ID NO: 1059 - RNA, 22 nt, Artificial Sequence
  B2M-targeting guide sequence portion
aauggagagagaauugaaaaag

SEQ ID NO: 1060 - RNA, 22 nt, Artificial Sequence
  B2M-targeting guide sequence portion
aaugucggauggaugaaaccca

SEQ ID NO: 1061 - RNA, 22 nt, Artificial Sequence
  B2M-targeting guide sequence portion
aauucacccccacugaaaaaga

SEQ ID NO: 1062 - RNA, 22 nt, Artificial Sequence
  B2M-targeting guide sequence portion
aauucaggaaauuugacuuucc

SEQ ID NO: 1063 - RNA, 22 nt, Artificial Sequence
  B2M-targeting guide sequence portion
aauucaguguaguacaagagau

SEQ ID NO: 1064 - RNA, 22 nt, Artificial Sequence
  B2M-targeting guide sequence portion
aauugaaaaaguggagcauuca

SEQ ID NO: 1065 - RNA, 22 nt, Artificial Sequence
  B2M-targeting guide sequence portion
aauugcuaugugucuggguuuc

SEQ ID NO: 1066 - RNA, 22 nt, Artificial Sequence
  B2M-targeting guide sequence portion
aauuuccugaauugcuaugugu

SEQ ID NO: 1067 - RNA, 22 nt, Artificial Sequence
  B2M-targeting guide sequence portion
aauuugacuuuccauucucugc

SEQ ID NO: 1068 - RNA, 22 nt, Artificial Sequence
  B2M-targeting guide sequence portion
acaaagucacaugguucacacg

SEQ ID NO: 1069 - RNA, 22 nt, Artificial Sequence
  B2M-targeting guide sequence portion
acaagagauagaaagaccaguc

SEQ ID NO: 1070 - RNA, 22 nt, Artificial Sequence
  B2M-targeting guide sequence portion
acaagucugaaugcuccacuuu

SEQ ID NO: 1071 - RNA, 22 nt, Artificial Sequence
  B2M-targeting guide sequence portion
acacauagcaauucaggaaauu

SEQ ID NO: 1072 - RNA, 22 nt, Artificial Sequence
  B2M-targeting guide sequence portion
acacggcaggcauacucaucuu

SEQ ID NO: 1073 - RNA, 22 nt, Artificial Sequence
  B2M-targeting guide sequence portion
acacugaauucacccccacuga

SEQ ID NO: 1074 - RNA, 22 nt, Artificial Sequence
  B2M-targeting guide sequence portion
acagcccaagauaguuaagugg

SEQ ID NO: 1075 - RNA, 22 nt, Artificial Sequence
  B2M-targeting guide sequence portion
acauagcaauucaggaaauuug

SEQ ID NO: 1076 - RNA, 22 nt, Artificial Sequence
  B2M-targeting guide sequence portion
acaugguucacacggcaggcau

SEQ ID NO: 1077 - RNA, 22 nt, Artificial Sequence
  B2M-targeting guide sequence portion
acauugaaguugacuuacugaa

SEQ ID NO: 1078 - RNA, 22 nt, Artificial Sequence
  B2M-targeting guide sequence portion
accaguccuugcugaaagacaa

SEQ ID NO: 1079 - RNA, 22 nt, Artificial Sequence
  B2M-targeting guide sequence portion
accaugugacuuugucacagcc

SEQ ID NO: 1080 - RNA, 22 nt, Artificial Sequence
  B2M-targeting guide sequence portion
acccagacacauagcaauucag

SEQ ID NO: 1081 - RNA, 22 nt, Artificial Sequence
  B2M-targeting guide sequence portion
accccacuuaacuaucuugggc

SEQ ID NO: 1082 - RNA, 22 nt, Artificial Sequence
  B2M-targeting guide sequence portion
acccccacugaaaaagaugagu

SEQ ID NO: 1083 - RNA, 22 nt, Artificial Sequence
  B2M-targeting guide sequence portion
acggcaggcauacucaucuuuu

SEQ ID NO: 1084 - RNA, 22 nt, Artificial Sequence
  B2M-targeting guide sequence portion
acgugaguaaaccugaaucuuu

SEQ ID NO: 1085 - RNA, 22 nt, Artificial Sequence
  B2M-targeting guide sequence portion
acuacacugaauucacccccac

SEQ ID NO: 1086 - RNA, 22 nt, Artificial Sequence
  B2M-targeting guide sequence portion
acuaucuugggcugugacaaag

SEQ ID NO: 1087 - RNA, 22 nt, Artificial Sequence
  B2M-targeting guide sequence portion
acucacgucauccagcagagaa

SEQ ID NO: 1088 - RNA, 22 nt, Artificial Sequence
  B2M-targeting guide sequence portion
acucaucuuuuucagugggggu

SEQ ID NO: 1089 - RNA, 22 nt, Artificial Sequence
  B2M-targeting guide sequence portion
acuccaaagauucagguuuacu

SEQ ID NO: 1090 - RNA, 22 nt, Artificial Sequence
  B2M-targeting guide sequence portion
acugaaaaagaugaguaugccu

SEQ ID NO: 1091 - RNA, 22 nt, Artificial Sequence
  B2M-targeting guide sequence portion
acugaagaauggagagagaauu

SEQ ID NO: 1092 - RNA, 22 nt, Artificial Sequence
  B2M-targeting guide sequence portion
acugaauucacccccacugaaa

SEQ ID NO: 1093 - RNA, 22 nt, Artificial Sequence
  B2M-targeting guide sequence portion
acuggucuuucuaucucuugua

SEQ ID NO: 1094 - RNA, 22 nt, Artificial Sequence
  B2M-targeting guide sequence portion
acuuaacuaucuugggcuguga

SEQ ID NO: 1095 - RNA, 22 nt, Artificial Sequence
  B2M-targeting guide sequence portion
acuuaccccacuuaacuaucuu

SEQ ID NO: 1096 - RNA, 22 nt, Artificial Sequence
  B2M-targeting guide sequence portion
acuuacugaagaauggagagag

SEQ ID NO: 1097 - RNA, 22 nt, Artificial Sequence
  B2M-targeting guide sequence portion
acuucaaugucggauggaugaa

SEQ ID NO: 1098 - RNA, 22 nt, Artificial Sequence
  B2M-targeting guide sequence portion
acuugucuuucagcaaggacug

SEQ ID NO: 1099 - RNA, 22 nt, Artificial Sequence
  B2M-targeting guide sequence portion
acuuugucacagcccaagauag

SEQ ID NO: 1100 - RNA, 22 nt, Artificial Sequence
  B2M-targeting guide sequence portion
acuuuuucaauucucucuccau

SEQ ID NO: 1101 - RNA, 22 nt, Artificial Sequence
  B2M-targeting guide sequence portion
agaaagaccaguccuugcugaa

SEQ ID NO: 1102 - RNA, 22 nt, Artificial Sequence
  B2M-targeting guide sequence portion
agaauggaaagucaaauuuccu

SEQ ID NO: 1103 - RNA, 22 nt, Artificial Sequence
  B2M-targeting guide sequence portion
agaauggagagagaauugaaaa

SEQ ID NO: 1104 - RNA, 22 nt, Artificial Sequence
  B2M-targeting guide sequence portion
agaauugaaaaaguggagcauu

SEQ ID NO: 1105 - RNA, 22 nt, Artificial Sequence
  B2M-targeting guide sequence portion
agacaagucugaaugcuccacu

SEQ ID NO: 1106 - RNA, 22 nt, Artificial Sequence
  B2M-targeting guide sequence portion
agacacauagcaauucaggaaa

SEQ ID NO: 1107 - RNA, 22 nt, Artificial Sequence
  B2M-targeting guide sequence portion
agaccaguccuugcugaaagac

SEQ ID NO: 1108 - RNA, 22 nt, Artificial Sequence
  B2M-targeting guide sequence portion
agacuugucuuucagcaaggac

SEQ ID NO: 1109 - RNA, 22 nt, Artificial Sequence
  B2M-targeting guide sequence portion
agagaauggaaagucaaauuuc

SEQ ID NO: 1110 - RNA, 22 nt, Artificial Sequence
  B2M-targeting guide sequence portion
agagaauugaaaaaguggagca

SEQ ID NO: 1111 - RNA, 22 nt, Artificial Sequence
  B2M-targeting guide sequence portion
agagagaauugaaaaaguggag

SEQ ID NO: 1112 - RNA, 22 nt, Artificial Sequence
  B2M-targeting guide sequence portion
agagauagaaagaccaguccuu

SEQ ID NO: 1113 - RNA, 22 nt, Artificial Sequence
  B2M-targeting guide sequence portion
agauagaaagaccaguccuugc

SEQ ID NO: 1114 - RNA, 22 nt, Artificial Sequence
  B2M-targeting guide sequence portion
agaugaguaugccugccgugug

SEQ ID NO: 1115 - RNA, 22 nt, Artificial Sequence
  B2M-targeting guide sequence portion
agcaauucaggaaauuugacuu

SEQ ID NO: 1116 - RNA, 22 nt, Artificial Sequence
  B2M-targeting guide sequence portion
agcagagaauggaaagucaaau

SEQ ID NO: 1117 - RNA, 22 nt, Artificial Sequence
  B2M-targeting guide sequence portion
agcauucagacuugucuuucag

SEQ ID NO: 1118 - RNA, 22 nt, Artificial Sequence
  B2M-targeting guide sequence portion
agcccaagauaguuaagugggg

SEQ ID NO: 1119 - RNA, 22 nt, Artificial Sequence
  B2M-targeting guide sequence portion
aggaaauuugacuuuccauucu

SEQ ID NO: 1120 - RNA, 22 nt, Artificial Sequence
  B2M-targeting guide sequence portion
aggacuggucuuucuaucucuu

SEQ ID NO: 1121 - RNA, 22 nt, Artificial Sequence
  B2M-targeting guide sequence portion
aggcauacucaucuuuuucagu

SEQ ID NO: 1122 - RNA, 22 nt, Artificial Sequence
  B2M-targeting guide sequence portion
agguacuccaaagauucagguu

SEQ ID NO: 1123 - RNA, 22 nt, Artificial Sequence
  B2M-targeting guide sequence portion
agguuuacucacgucauccagc

SEQ ID NO: 1124 - RNA, 22 nt, Artificial Sequence
  B2M-targeting guide sequence portion
aguaaaccugaaucuuuggagu

SEQ ID NO: 1125 - RNA, 22 nt, Artificial Sequence
  B2M-targeting guide sequence portion
aguaagucaacuucaaugucgg

SEQ ID NO: 1126 - RNA, 22 nt, Artificial Sequence
  B2M-targeting guide sequence portion
aguacaagagauagaaagacca

SEQ ID NO: 1127 - RNA, 22 nt, Artificial Sequence
  B2M-targeting guide sequence portion
aguaugccugccgugugaacca

SEQ ID NO: 1128 - RNA, 22 nt, Artificial Sequence
  B2M-targeting guide sequence portion
agucaaauuuccugaauugcua

SEQ ID NO: 1129 - RNA, 22 nt, Artificial Sequence
  B2M-targeting guide sequence portion
agucacaugguucacacggcag

SEQ ID NO: 1130 - RNA, 22 nt, Artificial Sequence
  B2M-targeting guide sequence portion
aguccuugcugaaagacaaguc

SEQ ID NO: 1131 - RNA, 22 nt, Artificial Sequence
  B2M-targeting guide sequence portion
agucugaaugcuccacuuuuuc

SEQ ID NO: 1132 - RNA, 22 nt, Artificial Sequence
  B2M-targeting guide sequence portion
aguggagcauucagacuugucu

SEQ ID NO: 1133 - RNA, 22 nt, Artificial Sequence
  B2M-targeting guide sequence portion
aguguaguacaagagauagaaa

SEQ ID NO: 1134 - RNA, 22 nt, Artificial Sequence
  B2M-targeting guide sequence portion
aguugacuuacugaagaaugga

SEQ ID NO: 1135 - RNA, 22 nt, Artificial Sequence
  B2M-targeting guide sequence portion
auacucaucuuuuucagugggg

SEQ ID NO: 1136 - RNA, 22 nt, Artificial Sequence
  B2M-targeting guide sequence portion
auagaaagaccaguccuugcug

SEQ ID NO: 1137 - RNA, 22 nt, Artificial Sequence
  B2M-targeting guide sequence portion
auagcaauucaggaaauuugac

SEQ ID NO: 1138 - RNA, 22 nt, Artificial Sequence
  B2M-targeting guide sequence portion
auccagcagagaauggaaaguc

SEQ ID NO: 1139 - RNA, 22 nt, Artificial Sequence
  B2M-targeting guide sequence portion
auccgacauugaaguugacuua

SEQ ID NO: 1140 - RNA, 22 nt, Artificial Sequence
  B2M-targeting guide sequence portion
aucucuuguacuacacugaauu

SEQ ID NO: 1141 - RNA, 22 nt, Artificial Sequence
  B2M-targeting guide sequence portion
aucuugggcugugacaaaguca

SEQ ID NO: 1142 - RNA, 22 nt, Artificial Sequence
  B2M-targeting guide sequence portion
aucuuuuucagugggggugaau

SEQ ID NO: 1143 - RNA, 22 nt, Artificial Sequence
  B2M-targeting guide sequence portion
augaaacccagacacauagcaa

SEQ ID NO: 1144 - RNA, 22 nt, Artificial Sequence
  B2M-targeting guide sequence portion
augacgugaguaaaccugaauc

SEQ ID NO: 1145 - RNA, 22 nt, Artificial Sequence
  B2M-targeting guide sequence portion
augaguaugccugccgugugaa

SEQ ID NO: 1146 - RNA, 22 nt, Artificial Sequence
  B2M-targeting guide sequence portion
augccugccgugugaaccaugu

SEQ ID NO: 1147 - RNA, 22 nt, Artificial Sequence
  B2M-targeting guide sequence portion
augcuccacuuuuucaauucuc

SEQ ID NO: 1148 - RNA, 22 nt, Artificial Sequence
  B2M-targeting guide sequence portion
auggaaagucaaauuuccugaa

SEQ ID NO: 1149 - RNA, 22 nt, Artificial Sequence
  B2M-targeting guide sequence portion
auggagagagaauugaaaaagu

SEQ ID NO: 1150 - RNA, 22 nt, Artificial Sequence
  B2M-targeting guide sequence portion
auggaugaaacccagacacaua

SEQ ID NO: 1151 - RNA, 22 nt, Artificial Sequence
  B2M-targeting guide sequence portion
augguucacacggcaggcauac

SEQ ID NO: 1152 - RNA, 22 nt, Artificial Sequence
  B2M-targeting guide sequence portion
augucggauggaugaaacccag

SEQ ID NO: 1153 - RNA, 22 nt, Artificial Sequence
  B2M-targeting guide sequence portion
augugacuuugucacagcccaa

SEQ ID NO: 1154 - RNA, 22 nt, Artificial Sequence
  B2M-targeting guide sequence portion
augugucuggguuucauccauc

SEQ ID NO: 1155 - RNA, 22 nt, Artificial Sequence
  B2M-targeting guide sequence portion
auucacccccacugaaaaagau

SEQ ID NO: 1156 - RNA, 22 nt, Artificial Sequence
  B2M-targeting guide sequence portion
auucagacuugucuuucagcaa

SEQ ID NO: 1157 - RNA, 22 nt, Artificial Sequence
  B2M-targeting guide sequence portion
auucaggaaauuugacuuucca

SEQ ID NO: 1158 - RNA, 22 nt, Artificial Sequence
  B2M-targeting guide sequence portion
auucagguuuacucacgucauc

SEQ ID NO: 1159 - RNA, 22 nt, Artificial Sequence
  B2M-targeting guide sequence portion
auucaguguaguacaagagaua

SEQ ID NO: 1160 - RNA, 22 nt, Artificial Sequence
  B2M-targeting guide sequence portion
auucucucuccauucuucagua

SEQ ID NO: 1161 - RNA, 22 nt, Artificial Sequence
  B2M-targeting guide sequence portion
auucucugcuggaugacgugag

SEQ ID NO: 1162 - RNA, 22 nt, Artificial Sequence
  B2M-targeting guide sequence portion
auucuucaguaagucaacuuca

SEQ ID NO: 1163 - RNA, 22 nt, Artificial Sequence
  B2M-targeting guide sequence portion
auugaaaaaguggagcauucag

SEQ ID NO: 1164 - RNA, 22 nt, Artificial Sequence
  B2M-targeting guide sequence portion
auugaaguugacuuacugaaga

SEQ ID NO: 1165 - RNA, 22 nt, Artificial Sequence
  B2M-targeting guide sequence portion
auuuccugaauugcuauguguc

SEQ ID NO: 1166 - RNA, 22 nt, Artificial Sequence
  B2M-targeting guide sequence portion
caaagauucagguuuacucacg

SEQ ID NO: 1167 - RNA, 22 nt, Artificial Sequence
  B2M-targeting guide sequence portion
caaagucacaugguucacacgg

SEQ ID NO: 1168 - RNA, 22 nt, Artificial Sequence
  B2M-targeting guide sequence portion
caaauuuccugaauugcuaugu

SEQ ID NO: 1169 - RNA, 22 nt, Artificial Sequence
  B2M-targeting guide sequence portion
caacuucaaugucggauggaug

SEQ ID NO: 1170 - RNA, 22 nt, Artificial Sequence
  B2M-targeting guide sequence portion
caagagauagaaagaccagucc

SEQ ID NO: 1171 - RNA, 22 nt, Artificial Sequence
  B2M-targeting guide sequence portion
caaggacuggucuuucuaucuc

SEQ ID NO: 1172 - RNA, 22 nt, Artificial Sequence
  B2M-targeting guide sequence portion
caagucugaaugcuccacuuuu

SEQ ID NO: 1173 - RNA, 22 nt, Artificial Sequence
  B2M-targeting guide sequence portion
caaugucggauggaugaaaccc

SEQ ID NO: 1174 - RNA, 22 nt, Artificial Sequence
  B2M-targeting guide sequence portion
caauucaggaaauuugacuuuc

SEQ ID NO: 1175 - RNA, 22 nt, Artificial Sequence
  B2M-targeting guide sequence portion
cacacggcaggcauacucaucu

SEQ ID NO: 1176 - RNA, 22 nt, Artificial Sequence
  B2M-targeting guide sequence portion
cacauagcaauucaggaaauuu

SEQ ID NO: 1177 - RNA, 22 nt, Artificial Sequence
  B2M-targeting guide sequence portion
cacaugguucacacggcaggca

SEQ ID NO: 1178 - RNA, 22 nt, Artificial Sequence
  B2M-targeting guide sequence portion
cacccccacugaaaaagaugag

SEQ ID NO: 1179 - RNA, 22 nt, Artificial Sequence
  B2M-targeting guide sequence portion
cacggcaggcauacucaucuuu

SEQ ID NO: 1180 - RNA, 22 nt, Artificial Sequence
  B2M-targeting guide sequence portion
cacgucauccagcagagaaugg

SEQ ID NO: 1181 - RNA, 22 nt, Artificial Sequence
  B2M-targeting guide sequence portion
cacugaaaaagaugaguaugcc

SEQ ID NO: 1182 - RNA, 22 nt, Artificial Sequence
  B2M-targeting guide sequence portion
cacugaauucacccccacugaa

SEQ ID NO: 1183 - RNA, 22 nt, Artificial Sequence
  B2M-targeting guide sequence portion
cacuuaacuaucuugggcugug

SEQ ID NO: 1184 - RNA, 22 nt, Artificial Sequence
  B2M-targeting guide sequence portion
cacuuuuucaauucucucucca

SEQ ID NO: 1185 - RNA, 22 nt, Artificial Sequence
  B2M-targeting guide sequence portion
cagacacauagcaauucaggaa

SEQ ID NO: 1186 - RNA, 22 nt, Artificial Sequence
  B2M-targeting guide sequence portion
cagacuugucuuucagcaagga

SEQ ID NO: 1187 - RNA, 22 nt, Artificial Sequence
  B2M-targeting guide sequence portion
cagagaauggaaagucaaauuu

SEQ ID NO: 1188 - RNA, 22 nt, Artificial Sequence
  B2M-targeting guide sequence portion
cagcaaggacuggucuuucuau

SEQ ID NO: 1189 - RNA, 22 nt, Artificial Sequence
  B2M-targeting guide sequence portion
cagcagagaauggaaagucaaa

SEQ ID NO: 1190 - RNA, 22 nt, Artificial Sequence
  B2M-targeting guide sequence portion
cagcccaagauaguuaaguggg

SEQ ID NO: 1191 - RNA, 22 nt, Artificial Sequence
  B2M-targeting guide sequence portion
caggcauacucaucuuuuucag

SEQ ID NO: 1192 - RNA, 22 nt, Artificial Sequence
  B2M-targeting guide sequence portion
cagguacuccaaagauucaggu

SEQ ID NO: 1193 - RNA, 22 nt, Artificial Sequence
  B2M-targeting guide sequence portion
caguaagucaacuucaaugucg

SEQ ID NO: 1194 - RNA, 22 nt, Artificial Sequence
  B2M-targeting guide sequence portion
caguccuugcugaaagacaagu

SEQ ID NO: 1195 - RNA, 22 nt, Artificial Sequence
  B2M-targeting guide sequence portion
caguguaguacaagagauagaa

SEQ ID NO: 1196 - RNA, 22 nt, Artificial Sequence
  B2M-targeting guide sequence portion
cauacucaucuuuuucaguggg

SEQ ID NO: 1197 - RNA, 22 nt, Artificial Sequence
  B2M-targeting guide sequence portion
cauagcaauucaggaaauuuga

SEQ ID NO: 1198 - RNA, 22 nt, Artificial Sequence
  B2M-targeting guide sequence portion
cauccagcagagaauggaaagu

SEQ ID NO: 1199 - RNA, 22 nt, Artificial Sequence
  B2M-targeting guide sequence portion
cauccauccgacauugaaguug

SEQ ID NO: 1200 - RNA, 22 nt, Artificial Sequence
  B2M-targeting guide sequence portion
cauccgacauugaaguugacuu

SEQ ID NO: 1201 - RNA, 22 nt, Artificial Sequence
  B2M-targeting guide sequence portion
caucuuuuucagugggggugaa

SEQ ID NO: 1202 - RNA, 22 nt, Artificial Sequence
  B2M-targeting guide sequence portion
caugguucacacggcaggcaua

SEQ ID NO: 1203 - RNA, 22 nt, Artificial Sequence
  B2M-targeting guide sequence portion
caugugacuuugucacagccca

SEQ ID NO: 1204 - RNA, 22 nt, Artificial Sequence
  B2M-targeting guide sequence portion
cauucagacuugucuuucagca

SEQ ID NO: 1205 - RNA, 22 nt, Artificial Sequence
  B2M-targeting guide sequence portion
cauucuucaguaagucaacuuc

SEQ ID NO: 1206 - RNA, 22 nt, Artificial Sequence
  B2M-targeting guide sequence portion
cauugaaguugacuuacugaag

SEQ ID NO: 1207 - RNA, 22 nt, Artificial Sequence
  B2M-targeting guide sequence portion
ccaaagauucagguuuacucac

SEQ ID NO: 1208 - RNA, 22 nt, Artificial Sequence
  B2M-targeting guide sequence portion
ccacugaaaaagaugaguaugc

SEQ ID NO: 1209 - RNA, 22 nt, Artificial Sequence
  B2M-targeting guide sequence portion
ccacuuaacuaucuugggcugu

SEQ ID NO: 1210 - RNA, 22 nt, Artificial Sequence
  B2M-targeting guide sequence portion
ccacuuuuucaauucucucucc

SEQ ID NO: 1211 - RNA, 22 nt, Artificial Sequence
  B2M-targeting guide sequence portion
ccagacacauagcaauucagga

SEQ ID NO: 1212 - RNA, 22 nt, Artificial Sequence
  B2M-targeting guide sequence portion
ccaguccuugcugaaagacaag

SEQ ID NO: 1213 - RNA, 22 nt, Artificial Sequence
  B2M-targeting guide sequence portion
ccauccgacauugaaguugacu

SEQ ID NO: 1214 - RNA, 22 nt, Artificial Sequence
  B2M-targeting guide sequence portion
ccaugugacuuugucacagccc

SEQ ID NO: 1215 - RNA, 22 nt, Artificial Sequence
  B2M-targeting guide sequence portion
ccauucucugcuggaugacgug

SEQ ID NO: 1216 - RNA, 22 nt, Artificial Sequence
  B2M-targeting guide sequence portion
ccauucuucaguaagucaacuu

SEQ ID NO: 1217 - RNA, 22 nt, Artificial Sequence
  B2M-targeting guide sequence portion
cccacugaaaaagaugaguaug

SEQ ID NO: 1218 - RNA, 22 nt, Artificial Sequence
  B2M-targeting guide sequence portion
cccacuuaacuaucuugggcug

SEQ ID NO: 1219 - RNA, 22 nt, Artificial Sequence
  B2M-targeting guide sequence portion
ccccacugaaaaagaugaguau

SEQ ID NO: 1220 - RNA, 22 nt, Artificial Sequence
  B2M-targeting guide sequence portion
ccccacuuaacuaucuugggcu

SEQ ID NO: 1221 - RNA, 22 nt, Artificial Sequence
  B2M-targeting guide sequence portion
cccccacugaaaaagaugagua

SEQ ID NO: 1222 - RNA, 22 nt, Artificial Sequence
  B2M-targeting guide sequence portion
ccgacauugaaguugacuuacu

SEQ ID NO: 1223 - RNA, 22 nt, Artificial Sequence
  B2M-targeting guide sequence portion
ccgugugaaccaugugacuuug

SEQ ID NO: 1224 - RNA, 22 nt, Artificial Sequence
  B2M-targeting guide sequence portion
ccucagguacuccaaagauuca

SEQ ID NO: 1225 - RNA, 22 nt, Artificial Sequence
  B2M-targeting guide sequence portion
ccugaauugcuaugugucuggg

SEQ ID NO: 1226 - RNA, 22 nt, Artificial Sequence
  B2M-targeting guide sequence portion
ccugccgugugaaccaugugac

SEQ ID NO: 1227 - RNA, 22 nt, Artificial Sequence
  B2M-targeting guide sequence portion
ccuugcugaaagacaagucuga

SEQ ID NO: 1228 - RNA, 22 nt, Artificial Sequence
  B2M-targeting guide sequence portion
cgacauugaaguugacuuacug

SEQ ID NO: 1229 - RNA, 22 nt, Artificial Sequence
  B2M-targeting guide sequence portion
cggauggaugaaacccagacac

SEQ ID NO: 1230 - RNA, 22 nt, Artificial Sequence
  B2M-targeting guide sequence portion
cgucauccagcagagaauggaa

SEQ ID NO: 1231 - RNA, 22 nt, Artificial Sequence
  B2M-targeting guide sequence portion
cgugaguaaaccugaaucuuug

SEQ ID NO: 1232 - RNA, 22 nt, Artificial Sequence
  B2M-targeting guide sequence portion
cgugugaaccaugugacuuugu

SEQ ID NO: 1233 - RNA, 22 nt, Artificial Sequence
  B2M-targeting guide sequence portion
cuacacugaauucacccccacu

SEQ ID NO: 1234 - RNA, 22 nt, Artificial Sequence
  B2M-targeting guide sequence portion
cuaucuugggcugugacaaagu

SEQ ID NO: 1235 - RNA, 22 nt, Artificial Sequence
  B2M-targeting guide sequence portion
cuaugugucuggguuucaucca

SEQ ID NO: 1236 - RNA, 22 nt, Artificial Sequence
  B2M-targeting guide sequence portion
cucagguacuccaaagauucag

SEQ ID NO: 1237 - RNA, 22 nt, Artificial Sequence
  B2M-targeting guide sequence portion
cucaucuuuuucagugggggug

SEQ ID NO: 1238 - RNA, 22 nt, Artificial Sequence
  B2M-targeting guide sequence portion
cuccaaagauucagguuuacuc

SEQ ID NO: 1239 - RNA, 22 nt, Artificial Sequence
  B2M-targeting guide sequence portion
cuccacuuuuucaauucucucu

SEQ ID NO: 1240 - RNA, 22 nt, Artificial Sequence
  B2M-targeting guide sequence portion
cuccauucuucaguaagucaac

SEQ ID NO: 1241 - RNA, 22 nt, Artificial Sequence
  B2M-targeting guide sequence portion
cucuccauucuucaguaaguca

SEQ ID NO: 1242 - RNA, 22 nt, Artificial Sequence
  B2M-targeting guide sequence portion
cucucuccauucuucaguaagu

SEQ ID NO: 1243 - RNA, 22 nt, Artificial Sequence
  B2M-targeting guide sequence portion
cucuuguacuacacugaauuca

SEQ ID NO: 1244 - RNA, 22 nt, Artificial Sequence
  B2M-targeting guide sequence portion
cugaaaaagaugaguaugccug

SEQ ID NO: 1245 - RNA, 22 nt, Artificial Sequence
  B2M-targeting guide sequence portion
cugaaagacaagucugaaugcu

SEQ ID NO: 1246 - RNA, 22 nt, Artificial Sequence
  B2M-targeting guide sequence portion
cugaagaauggagagagaauug

SEQ ID NO: 1247 - RNA, 22 nt, Artificial Sequence
  B2M-targeting guide sequence portion
cugaaugcuccacuuuuucaau

SEQ ID NO: 1248 - RNA, 22 nt, Artificial Sequence
  B2M-targeting guide sequence portion
cugaauucacccccacugaaaa

SEQ ID NO: 1249 - RNA, 22 nt, Artificial Sequence
  B2M-targeting guide sequence portion
cugccgugugaaccaugugacu

SEQ ID NO: 1250 - RNA, 22 nt, Artificial Sequence
  B2M-targeting guide sequence portion
cugcuggaugacgugaguaaac

SEQ ID NO: 1251 - RNA, 22 nt, Artificial Sequence
  B2M-targeting guide sequence portion
cuggaugacgugaguaaaccug

SEQ ID NO: 1252 - RNA, 22 nt, Artificial Sequence
  B2M-targeting guide sequence portion
cuggguuucauccauccgacau

SEQ ID NO: 1253 - RNA, 22 nt, Artificial Sequence
  B2M-targeting guide sequence portion
cuggucuuucuaucucuuguac

SEQ ID NO: 1254 - RNA, 22 nt, Artificial Sequence
  B2M-targeting guide sequence portion
cugugacaaagucacaugguuc

SEQ ID NO: 1255 - RNA, 22 nt, Artificial Sequence
  B2M-targeting guide sequence portion
cuuaacuaucuugggcugugac

SEQ ID NO: 1256 - RNA, 22 nt, Artificial Sequence
  B2M-targeting guide sequence portion
cuuaccccacuuaacuaucuug

SEQ ID NO: 1257 - RNA, 22 nt, Artificial Sequence
  B2M-targeting guide sequence portion
cuuacugaagaauggagagaga

SEQ ID NO: 1258 - RNA, 22 nt, Artificial Sequence
  B2M-targeting guide sequence portion
cuucaaugucggauggaugaaa

SEQ ID NO: 1259 - RNA, 22 nt, Artificial Sequence
  B2M-targeting guide sequence portion
cuucaguaagucaacuucaaug

SEQ ID NO: 1260 - RNA, 22 nt, Artificial Sequence
  B2M-targeting guide sequence portion
cuugcugaaagacaagucugaa

SEQ ID NO: 1261 - RNA, 22 nt, Artificial Sequence
  B2M-targeting guide sequence portion
cuugggcugugacaaagucaca

SEQ ID NO: 1262 - RNA, 22 nt, Artificial Sequence
  B2M-targeting guide sequence portion
cuuguacuacacugaauucacc

SEQ ID NO: 1263 - RNA, 22 nt, Artificial Sequence
  B2M-targeting guide sequence portion
cuugucuuucagcaaggacugg

SEQ ID NO: 1264 - RNA, 22 nt, Artificial Sequence
  B2M-targeting guide sequence portion
cuuucagcaaggacuggucuuu

SEQ ID NO: 1265 - RNA, 22 nt, Artificial Sequence
  B2M-targeting guide sequence portion
cuuuccauucucugcuggauga

SEQ ID NO: 1266 - RNA, 22 nt, Artificial Sequence
  B2M-targeting guide sequence portion
cuuucuaucucuuguacuacac

SEQ ID NO: 1267 - RNA, 22 nt, Artificial Sequence
  B2M-targeting guide sequence portion
cuuugucacagcccaagauagu

SEQ ID NO: 1268 - RNA, 22 nt, Artificial Sequence
  B2M-targeting guide sequence portion
cuuuuucaauucucucuccauu

SEQ ID NO: 1269 - RNA, 22 nt, Artificial Sequence
  B2M-targeting guide sequence portion
cuuuuucagugggggugaauuc

SEQ ID NO: 1270 - RNA, 22 nt, Artificial Sequence
  B2M-targeting guide sequence portion
gaaaaagaugaguaugccugcc

SEQ ID NO: 1271 - RNA, 22 nt, Artificial Sequence
  B2M-targeting guide sequence portion
gaaaaaguggagcauucagacu

SEQ ID NO: 1272 - RNA, 22 nt, Artificial Sequence
  B2M-targeting guide sequence portion
gaaacccagacacauagcaauu

SEQ ID NO: 1273 - RNA, 22 nt, Artificial Sequence
  B2M-targeting guide sequence portion
gaaagacaagucugaaugcucc

SEQ ID NO: 1274 - RNA, 22 nt, Artificial Sequence
  B2M-targeting guide sequence portion
gaaagaccaguccuugcugaaa

SEQ ID NO: 1275 - RNA, 22 nt, Artificial Sequence
  B2M-targeting guide sequence portion
gaaagucaaauuuccugaauug

SEQ ID NO: 1276 - RNA, 22 nt, Artificial Sequence
  B2M-targeting guide sequence portion
gaaauuugacuuuccauucucu

SEQ ID NO: 1277 - RNA, 22 nt, Artificial Sequence
  B2M-targeting guide sequence portion
gaaccaugugacuuugucacag

SEQ ID NO: 1278 - RNA, 22 nt, Artificial Sequence
  B2M-targeting guide sequence portion
gaagaauggagagagaauugaa

SEQ ID NO: 1279 - RNA, 22 nt, Artificial Sequence
  B2M-targeting guide sequence portion
gaaguugacuuacugaagaaug

SEQ ID NO: 1280 - RNA, 22 nt, Artificial Sequence
  B2M-targeting guide sequence portion
gaaugcuccacuuuuucaauuc

SEQ ID NO: 1281 - RNA, 22 nt, Artificial Sequence
  B2M-targeting guide sequence portion
gaauggaaagucaaauuuccug

SEQ ID NO: 1282 - RNA, 22 nt, Artificial Sequence
  B2M-targeting guide sequence portion
gaauggagagagaauugaaaaa

SEQ ID NO: 1283 - RNA, 22 nt, Artificial Sequence
  B2M-targeting guide sequence portion
gaauucacccccacugaaaaag

SEQ ID NO: 1284 - RNA, 22 nt, Artificial Sequence
  B2M-targeting guide sequence portion
gaauucaguguaguacaagaga

SEQ ID NO: 1285 - RNA, 22 nt, Artificial Sequence
  B2M-targeting guide sequence portion
gaauugaaaaaguggagcauuc

SEQ ID NO: 1286 - RNA, 22 nt, Artificial Sequence
  B2M-targeting guide sequence portion
gaauugcuaugugucuggguuu

SEQ ID NO: 1287 - RNA, 22 nt, Artificial Sequence
  B2M-targeting guide sequence portion
gacaaagucacaugguucacac

SEQ ID NO: 1288 - RNA, 22 nt, Artificial Sequence
  B2M-targeting guide sequence portion
gacacauagcaauucaggaaau

SEQ ID NO: 1289 - RNA, 22 nt, Artificial Sequence
  B2M-targeting guide sequence portion
gacauugaaguugacuuacuga

SEQ ID NO: 1290 - RNA, 22 nt, Artificial Sequence
  B2M-targeting guide sequence portion
gacgugaguaaaccugaaucuu

SEQ ID NO: 1291 - RNA, 22 nt, Artificial Sequence
  B2M-targeting guide sequence portion
gacuggucuuucuaucucuugu

SEQ ID NO: 1292 - RNA, 22 nt, Artificial Sequence
  B2M-targeting guide sequence portion
gacuuaccccacuuaacuaucu

SEQ ID NO: 1293 - RNA, 22 nt, Artificial Sequence
  B2M-targeting guide sequence portion
gacuuacugaagaauggagaga

SEQ ID NO: 1294 - RNA, 22 nt, Artificial Sequence
  B2M-targeting guide sequence portion
gacuugucuuucagcaaggacu

SEQ ID NO: 1295 - RNA, 22 nt, Artificial Sequence
  B2M-targeting guide sequence portion
gacuuuccauucucugcuggau

SEQ ID NO: 1296 - RNA, 22 nt, Artificial Sequence
  B2M-targeting guide sequence portion
gacuuugucacagcccaagaua

SEQ ID NO: 1297 - RNA, 22 nt, Artificial Sequence
  B2M-targeting guide sequence portion
gagaauggaaagucaaauuucc

SEQ ID NO: 1298 - RNA, 22 nt, Artificial Sequence
  B2M-targeting guide sequence portion
gagaauugaaaaaguggagcau

SEQ ID NO: 1299 - RNA, 22 nt, Artificial Sequence
  B2M-targeting guide sequence portion
gagagaauugaaaaaguggagc

SEQ ID NO: 1300 - RNA, 22 nt, Artificial Sequence
  B2M-targeting guide sequence portion
gagagagaauugaaaaagugga

SEQ ID NO: 1301 - RNA, 22 nt, Artificial Sequence
  B2M-targeting guide sequence portion
gagauagaaagaccaguccuug

SEQ ID NO: 1302 - RNA, 22 nt, Artificial Sequence
  B2M-targeting guide sequence portion
gagcauucagacuugucuuuca

SEQ ID NO: 1303 - RNA, 22 nt, Artificial Sequence
  B2M-targeting guide sequence portion
gaguaaaccugaaucuuuggag

SEQ ID NO: 1304 - RNA, 22 nt, Artificial Sequence
  B2M-targeting guide sequence portion
gaguaugccugccgugugaacc

SEQ ID NO: 1305 - RNA, 22 nt, Artificial Sequence
  B2M-targeting guide sequence portion
gaugaaacccagacacauagca

SEQ ID NO: 1306 - RNA, 22 nt, Artificial Sequence
  B2M-targeting guide sequence portion
gaugacgugaguaaaccugaau

SEQ ID NO: 1307 - RNA, 22 nt, Artificial Sequence
  B2M-targeting guide sequence portion
gaugaguaugccugccguguga

SEQ ID NO: 1308 - RNA, 22 nt, Artificial Sequence
  B2M-targeting guide sequence portion
gauggaugaaacccagacacau

SEQ ID NO: 1309 - RNA, 22 nt, Artificial Sequence
  B2M-targeting guide sequence portion
gauucagguuuacucacgucau

SEQ ID NO: 1310 - RNA, 22 nt, Artificial Sequence
  B2M-targeting guide sequence portion
gcaaggacuggucuuucuaucu

SEQ ID NO: 1311 - RNA, 22 nt, Artificial Sequence
  B2M-targeting guide sequence portion
gcaauucaggaaauuugacuuu

SEQ ID NO: 1312 - RNA, 22 nt, Artificial Sequence
  B2M-targeting guide sequence portion
gcagagaauggaaagucaaauu

SEQ ID NO: 1313 - RNA, 22 nt, Artificial Sequence
  B2M-targeting guide sequence portion
gcaggcauacucaucuuuuuca

SEQ ID NO: 1314 - RNA, 22 nt, Artificial Sequence
  B2M-targeting guide sequence portion
gcauacucaucuuuuucagugg

SEQ ID NO: 1315 - RNA, 22 nt, Artificial Sequence
  B2M-targeting guide sequence portion
gcauucagacuugucuuucagc

SEQ ID NO: 1316 - RNA, 22 nt, Artificial Sequence
  B2M-targeting guide sequence portion
gccugccgugugaaccauguga

SEQ ID NO: 1317 - RNA, 22 nt, Artificial Sequence
  B2M-targeting guide sequence portion
gcuaugugucuggguuucaucc

SEQ ID NO: 1318 - RNA, 22 nt, Artificial Sequence
  B2M-targeting guide sequence portion
gcuccacuuuuucaauucucuc

SEQ ID NO: 1319 - RNA, 22 nt, Artificial Sequence
  B2M-targeting guide sequence portion
gcuggaugacgugaguaaaccu

SEQ ID NO: 1320 - RNA, 22 nt, Artificial Sequence
  B2M-targeting guide sequence portion
gcugugacaaagucacaugguu

SEQ ID NO: 1321 - RNA, 22 nt, Artificial Sequence
  B2M-targeting guide sequence portion
ggaaagucaaauuuccugaauu

SEQ ID NO: 1322 - RNA, 22 nt, Artificial Sequence
  B2M-targeting guide sequence portion
ggaaauuugacuuuccauucuc

SEQ ID NO: 1323 - RNA, 22 nt, Artificial Sequence
  B2M-targeting guide sequence portion
ggacuggucuuucuaucucuug

SEQ ID NO: 1324 - RNA, 22 nt, Artificial Sequence
  B2M-targeting guide sequence portion
ggagagagaauugaaaaagugg

SEQ ID NO: 1325 - RNA, 22 nt, Artificial Sequence
  B2M-targeting guide sequence portion
ggagcauucagacuugucuuuc

SEQ ID NO: 1326 - RNA, 22 nt, Artificial Sequence
  B2M-targeting guide sequence portion
ggaugaaacccagacacauagc

SEQ ID NO: 1327 - RNA, 22 nt, Artificial Sequence
  B2M-targeting guide sequence portion
ggaugacgugaguaaaccugaa

SEQ ID NO: 1328 - RNA, 22 nt, Artificial Sequence
  B2M-targeting guide sequence portion
ggauggaugaaacccagacaca

SEQ ID NO: 1329 - RNA, 22 nt, Artificial Sequence
  B2M-targeting guide sequence portion
ggcaggcauacucaucuuuuuc

SEQ ID NO: 1330 - RNA, 22 nt, Artificial Sequence
  B2M-targeting guide sequence portion
ggcauacucaucuuuuucagug

SEQ ID NO: 1331 - RNA, 22 nt, Artificial Sequence
  B2M-targeting guide sequence portion
ggcugugacaaagucacauggu

SEQ ID NO: 1332 - RNA, 22 nt, Artificial Sequence
  B2M-targeting guide sequence portion
gggcugugacaaagucacaugg

SEQ ID NO: 1333 - RNA, 22 nt, Artificial Sequence
  B2M-targeting guide sequence portion
gggggugaauucaguguaguac

SEQ ID NO: 1334 - RNA, 22 nt, Artificial Sequence
  B2M-targeting guide sequence portion
ggggugaauucaguguaguaca

SEQ ID NO: 1335 - RNA, 22 nt, Artificial Sequence
  B2M-targeting guide sequence portion
gggugaauucaguguaguacaa

SEQ ID NO: 1336 - RNA, 22 nt, Artificial Sequence
  B2M-targeting guide sequence portion
ggguuucauccauccgacauug

SEQ ID NO: 1337 - RNA, 22 nt, Artificial Sequence
  B2M-targeting guide sequence portion
gguacuccaaagauucagguuu

SEQ ID NO: 1338 - RNA, 22 nt, Artificial Sequence
  B2M-targeting guide sequence portion
ggucuuucuaucucuuguacua

SEQ ID NO: 1339 - RNA, 22 nt, Artificial Sequence
  B2M-targeting guide sequence portion
gguuuacucacgucauccagca

SEQ ID NO: 1340 - RNA, 22 nt, Artificial Sequence
  B2M-targeting guide sequence portion
gguuucauccauccgacauuga

SEQ ID NO: 1341 - RNA, 22 nt, Artificial Sequence
  B2M-targeting guide sequence portion
guaaaccugaaucuuuggagua

SEQ ID NO: 1342 - RNA, 22 nt, Artificial Sequence
  B2M-targeting guide sequence portion
guaagucaacuucaaugucgga

SEQ ID NO: 1343 - RNA, 22 nt, Artificial Sequence
  B2M-targeting guide sequence portion
guacaagagauagaaagaccag

SEQ ID NO: 1344 - RNA, 22 nt, Artificial Sequence
  B2M-targeting guide sequence portion
guacuacacugaauucaccccc

SEQ ID NO: 1345 - RNA, 22 nt, Artificial Sequence
  B2M-targeting guide sequence portion
guacuccaaagauucagguuua

SEQ ID NO: 1346 - RNA, 22 nt, Artificial Sequence
  B2M-targeting guide sequence portion
guaguacaagagauagaaagac

SEQ ID NO: 1347 - RNA, 22 nt, Artificial Sequence
  B2M-targeting guide sequence portion
guaugccugccgugugaaccau

SEQ ID NO: 1348 - RNA, 22 nt, Artificial Sequence
  B2M-targeting guide sequence portion
gucaaauuuccugaauugcuau

SEQ ID NO: 1349 - RNA, 22 nt, Artificial Sequence
  B2M-targeting guide sequence portion
gucaacuucaaugucggaugga

SEQ ID NO: 1350 - RNA, 22 nt, Artificial Sequence
  B2M-targeting guide sequence portion
gucacagcccaagauaguuaag

SEQ ID NO: 1351 - RNA, 22 nt, Artificial Sequence
  B2M-targeting guide sequence portion
gucacaugguucacacggcagg

SEQ ID NO: 1352 - RNA, 22 nt, Artificial Sequence
  B2M-targeting guide sequence portion
gucauccagcagagaauggaaa

SEQ ID NO: 1353 - RNA, 22 nt, Artificial Sequence
  B2M-targeting guide sequence portion
guccuugcugaaagacaagucu

SEQ ID NO: 1354 - RNA, 22 nt, Artificial Sequence
  B2M-targeting guide sequence portion
gucggauggaugaaacccagac

SEQ ID NO: 1355 - RNA, 22 nt, Artificial Sequence
  B2M-targeting guide sequence portion
gucugaaugcuccacuuuuuca

SEQ ID NO: 1356 - RNA, 22 nt, Artificial Sequence
  B2M-targeting guide sequence portion
gucuggguuucauccauccgac

SEQ ID NO: 1357 - RNA, 22 nt, Artificial Sequence
  B2M-targeting guide sequence portion
gucuuucagcaaggacuggucu

SEQ ID NO: 1358 - RNA, 22 nt, Artificial Sequence
  B2M-targeting guide sequence portion
gucuuucuaucucuuguacuac

SEQ ID NO: 1359 - RNA, 22 nt, Artificial Sequence
  B2M-targeting guide sequence portion
gugaaccaugugacuuugucac

SEQ ID NO: 1360 - RNA, 22 nt, Artificial Sequence
  B2M-targeting guide sequence portion
gugaauucaguguaguacaaga

SEQ ID NO: 1361 - RNA, 22 nt, Artificial Sequence
  B2M-targeting guide sequence portion
gugacaaagucacaugguucac

SEQ ID NO: 1362 - RNA, 22 nt, Artificial Sequence
  B2M-targeting guide sequence portion
gugaguaaaccugaaucuuugg

SEQ ID NO: 1363 - RNA, 22 nt, Artificial Sequence
  B2M-targeting guide sequence portion
guggagcauucagacuugucuu

SEQ ID NO: 1364 - RNA, 22 nt, Artificial Sequence
  B2M-targeting guide sequence portion
gugggggugaauucaguguagu

SEQ ID NO: 1365 - RNA, 22 nt, Artificial Sequence
  B2M-targeting guide sequence portion
guguaguacaagagauagaaag

SEQ ID NO: 1366 - RNA, 22 nt, Artificial Sequence
  B2M-targeting guide sequence portion
guucacacggcaggcauacuca

SEQ ID NO: 1367 - RNA, 22 nt, Artificial Sequence
  B2M-targeting guide sequence portion
guugacuuacugaagaauggag

SEQ ID NO: 1368 - RNA, 22 nt, Artificial Sequence
  B2M-targeting guide sequence portion
guuuacucacgucauccagcag

SEQ ID NO: 1369 - RNA, 22 nt, Artificial Sequence
  B2M-targeting guide sequence portion
guuucauccauccgacauugaa

SEQ ID NO: 1370 - RNA, 22 nt, Artificial Sequence
  B2M-targeting guide sequence portion
uaaaccugaaucuuuggaguac

SEQ ID NO: 1371 - RNA, 22 nt, Artificial Sequence
  B2M-targeting guide sequence portion
uaacuaucuugggcugugacaa

SEQ ID NO: 1372 - RNA, 22 nt, Artificial Sequence
  B2M-targeting guide sequence portion
uaagucaacuucaaugucggau

SEQ ID NO: 1373 - RNA, 22 nt, Artificial Sequence
  B2M-targeting guide sequence portion
uacaagagauagaaagaccagu

SEQ ID NO: 1374 - RNA, 22 nt, Artificial Sequence
  B2M-targeting guide sequence portion
uacacugaauucacccccacug

SEQ ID NO: 1375 - RNA, 22 nt, Artificial Sequence
  B2M-targeting guide sequence portion
uaccccacuuaacuaucuuggg

SEQ ID NO: 1376 - RNA, 22 nt, Artificial Sequence
  B2M-targeting guide sequence portion
uacuacacugaauucaccccca

SEQ ID NO: 1377 - RNA, 22 nt, Artificial Sequence
  B2M-targeting guide sequence portion
uacucacgucauccagcagaga

SEQ ID NO: 1378 - RNA, 22 nt, Artificial Sequence
  B2M-targeting guide sequence portion
uacucaucuuuuucaguggggg

SEQ ID NO: 1379 - RNA, 22 nt, Artificial Sequence
  B2M-targeting guide sequence portion
uacuccaaagauucagguuuac

SEQ ID NO: 1380 - RNA, 22 nt, Artificial Sequence
  B2M-targeting guide sequence portion
uacugaagaauggagagagaau

SEQ ID NO: 1381 - RNA, 22 nt, Artificial Sequence
  B2M-targeting guide sequence portion
uagaaagaccaguccuugcuga

SEQ ID NO: 1382 - RNA, 22 nt, Artificial Sequence
  B2M-targeting guide sequence portion
uagcaauucaggaaauuugacu

SEQ ID NO: 1383 - RNA, 22 nt, Artificial Sequence
  B2M-targeting guide sequence portion
uaguacaagagauagaaagacc

SEQ ID NO: 1384 - RNA, 22 nt, Artificial Sequence
  B2M-targeting guide sequence portion
uaucuugggcugugacaaaguc

SEQ ID NO: 1385 - RNA, 22 nt, Artificial Sequence
  B2M-targeting guide sequence portion
uaugccugccgugugaaccaug

SEQ ID NO: 1386 - RNA, 22 nt, Artificial Sequence
  B2M-targeting guide sequence portion
uaugugucuggguuucauccau

SEQ ID NO: 1387 - RNA, 22 nt, Artificial Sequence
  B2M-targeting guide sequence portion
ucaaauuuccugaauugcuaug

SEQ ID NO: 1388 - RNA, 22 nt, Artificial Sequence
  B2M-targeting guide sequence portion
ucaacuucaaugucggauggau

SEQ ID NO: 1389 - RNA, 22 nt, Artificial Sequence
  B2M-targeting guide sequence portion
ucaaugucggauggaugaaacc

SEQ ID NO: 1390 - RNA, 22 nt, Artificial Sequence
  B2M-targeting guide sequence portion
ucacaugguucacacggcaggc

SEQ ID NO: 1391 - RNA, 22 nt, Artificial Sequence
  B2M-targeting guide sequence portion
ucacccccacugaaaaagauga

SEQ ID NO: 1392 - RNA, 22 nt, Artificial Sequence
  B2M-targeting guide sequence portion
ucacgucauccagcagagaaug

SEQ ID NO: 1393 - RNA, 22 nt, Artificial Sequence
  B2M-targeting guide sequence portion
ucagacuugucuuucagcaagg

SEQ ID NO: 1394 - RNA, 22 nt, Artificial Sequence
  B2M-targeting guide sequence portion
ucagcaaggacuggucuuucua

SEQ ID NO: 1395 - RNA, 22 nt, Artificial Sequence
  B2M-targeting guide sequence portion
ucaggaaauuugacuuuccauu

SEQ ID NO: 1396 - RNA, 22 nt, Artificial Sequence
  B2M-targeting guide sequence portion
ucagguacuccaaagauucagg

SEQ ID NO: 1397 - RNA, 22 nt, Artificial Sequence
  B2M-targeting guide sequence portion
ucagguuuacucacgucaucca

SEQ ID NO: 1398 - RNA, 22 nt, Artificial Sequence
  B2M-targeting guide sequence portion
ucaguaagucaacuucaauguc

SEQ ID NO: 1399 - RNA, 22 nt, Artificial Sequence
  B2M-targeting guide sequence portion
ucaguguaguacaagagauaga

SEQ ID NO: 1400 - RNA, 22 nt, Artificial Sequence
  B2M-targeting guide sequence portion
ucauccagcagagaauggaaag

SEQ ID NO: 1401 - RNA, 22 nt, Artificial Sequence
  B2M-targeting guide sequence portion
ucauccauccgacauugaaguu

SEQ ID NO: 1402 - RNA, 22 nt, Artificial Sequence
  B2M-targeting guide sequence portion
ucaucuuuuucaguggggguga

SEQ ID NO: 1403 - RNA, 22 nt, Artificial Sequence
  B2M-targeting guide sequence portion
uccaaagauucagguuuacuca

SEQ ID NO: 1404 - RNA, 22 nt, Artificial Sequence
  B2M-targeting guide sequence portion
uccacuuuuucaauucucucuc

SEQ ID NO: 1405 - RNA, 22 nt, Artificial Sequence
  B2M-targeting guide sequence portion
uccagcagagaauggaaaguca

SEQ ID NO: 1406 - RNA, 22 nt, Artificial Sequence
  B2M-targeting guide sequence portion
uccauccgacauugaaguugac

SEQ ID NO: 1407 - RNA, 22 nt, Artificial Sequence
  B2M-targeting guide sequence portion
uccauucucugcuggaugacgu

SEQ ID NO: 1408 - RNA, 22 nt, Artificial Sequence
  B2M-targeting guide sequence portion
uccauucuucaguaagucaacu

SEQ ID NO: 1409 - RNA, 22 nt, Artificial Sequence
  B2M-targeting guide sequence portion
uccgacauugaaguugacuuac

SEQ ID NO: 1410 - RNA, 22 nt, Artificial Sequence
  B2M-targeting guide sequence portion
uccucagguacuccaaagauuc

SEQ ID NO: 1411 - RNA, 22 nt, Artificial Sequence
  B2M-targeting guide sequence portion
uccugaauugcuaugugucugg

SEQ ID NO: 1412 - RNA, 22 nt, Artificial Sequence
  B2M-targeting guide sequence portion
ucggauggaugaaacccagaca

SEQ ID NO: 1413 - RNA, 22 nt, Artificial Sequence
  B2M-targeting guide sequence portion
ucuaucucuuguacuacacuga

SEQ ID NO: 1414 - RNA, 22 nt, Artificial Sequence
  B2M-targeting guide sequence portion
ucuccauucuucaguaagucaa

SEQ ID NO: 1415 - RNA, 22 nt, Artificial Sequence
  B2M-targeting guide sequence portion
ucucuccauucuucaguaaguc

SEQ ID NO: 1416 - RNA, 22 nt, Artificial Sequence
  B2M-targeting guide sequence portion
ucucucuccauucuucaguaag

SEQ ID NO: 1417 - RNA, 22 nt, Artificial Sequence
  B2M-targeting guide sequence portion
ucucugcuggaugacgugagua

SEQ ID NO: 1418 - RNA, 22 nt, Artificial Sequence
  B2M-targeting guide sequence portion
ucucuuguacuacacugaauuc

SEQ ID NO: 1419 - RNA, 22 nt, Artificial Sequence
  B2M-targeting guide sequence portion
ucugaaugcuccacuuuuucaa

SEQ ID NO: 1420 - RNA, 22 nt, Artificial Sequence
  B2M-targeting guide sequence portion
ucugcuggaugacgugaguaaa

SEQ ID NO: 1421 - RNA, 22 nt, Artificial Sequence
  B2M-targeting guide sequence portion
ucuggguuucauccauccgaca

SEQ ID NO: 1422 - RNA, 22 nt, Artificial Sequence
  B2M-targeting guide sequence portion
ucuucaguaagucaacuucaau

SEQ ID NO: 1423 - RNA, 22 nt, Artificial Sequence
  B2M-targeting guide sequence portion
ucuugggcugugacaaagucac

SEQ ID NO: 1424 - RNA, 22 nt, Artificial Sequence
  B2M-targeting guide sequence portion
ucuuguacuacacugaauucac

SEQ ID NO: 1425 - RNA, 22 nt, Artificial Sequence
  B2M-targeting guide sequence portion
ucuuucagcaaggacuggucuu

SEQ ID NO: 1426 - RNA, 22 nt, Artificial Sequence
  B2M-targeting guide sequence portion
ucuuucuaucucuuguacuaca

SEQ ID NO: 1427 - RNA, 22 nt, Artificial Sequence
  B2M-targeting guide sequence portion
ucuuuuucagugggggugaauu

SEQ ID NO: 1428 - RNA, 22 nt, Artificial Sequence
  B2M-targeting guide sequence portion
ugaaaaagaugaguaugccugc

SEQ ID NO: 1429 - RNA, 22 nt, Artificial Sequence
  B2M-targeting guide sequence portion
ugaaaaaguggagcauucagac

SEQ ID NO: 1430 - RNA, 22 nt, Artificial Sequence
  B2M-targeting guide sequence portion
ugaaacccagacacauagcaau

SEQ ID NO: 1431 - RNA, 22 nt, Artificial Sequence
  B2M-targeting guide sequence portion
ugaaagacaagucugaaugcuc

SEQ ID NO: 1432 - RNA, 22 nt, Artificial Sequence
  B2M-targeting guide sequence portion
ugaaccaugugacuuugucaca

SEQ ID NO: 1433 - RNA, 22 nt, Artificial Sequence
  B2M-targeting guide sequence portion
ugaagaauggagagagaauuga

SEQ ID NO: 1434 - RNA, 22 nt, Artificial Sequence
  B2M-targeting guide sequence portion
ugaaguugacuuacugaagaau

SEQ ID NO: 1435 - RNA, 22 nt, Artificial Sequence
  B2M-targeting guide sequence portion
ugaaugcuccacuuuuucaauu

SEQ ID NO: 1436 - RNA, 22 nt, Artificial Sequence
  B2M-targeting guide sequence portion
ugaauucacccccacugaaaaa

SEQ ID NO: 1437 - RNA, 22 nt, Artificial Sequence
  B2M-targeting guide sequence portion
ugaauucaguguaguacaagag

SEQ ID NO: 1438 - RNA, 22 nt, Artificial Sequence
  B2M-targeting guide sequence portion
ugaauugcuaugugucuggguu

SEQ ID NO: 1439 - RNA, 22 nt, Artificial Sequence
  B2M-targeting guide sequence portion
ugacaaagucacaugguucaca

SEQ ID NO: 1440 - RNA, 22 nt, Artificial Sequence
  B2M-targeting guide sequence portion
ugacgugaguaaaccugaaucu

SEQ ID NO: 1441 - RNA, 22 nt, Artificial Sequence
  B2M-targeting guide sequence portion
ugacuuacugaagaauggagag

SEQ ID NO: 1442 - RNA, 22 nt, Artificial Sequence
  B2M-targeting guide sequence portion
ugacuuuccauucucugcugga

SEQ ID NO: 1443 - RNA, 22 nt, Artificial Sequence
  B2M-targeting guide sequence portion
ugacuuugucacagcccaagau

SEQ ID NO: 1444 - RNA, 22 nt, Artificial Sequence
  B2M-targeting guide sequence portion
ugaguaaaccugaaucuuugga

SEQ ID NO: 1445 - RNA, 22 nt, Artificial Sequence
  B2M-targeting guide sequence portion
ugaguaugccugccgugugaac

SEQ ID NO: 1446 - RNA, 22 nt, Artificial Sequence
  B2M-targeting guide sequence portion
ugccugccgugugaaccaugug

SEQ ID NO: 1447 - RNA, 22 nt, Artificial Sequence
  B2M-targeting guide sequence portion
ugcuaugugucuggguuucauc

SEQ ID NO: 1448 - RNA, 22 nt, Artificial Sequence
  B2M-targeting guide sequence portion
ugcuccacuuuuucaauucucu

SEQ ID NO: 1449 - RNA, 22 nt, Artificial Sequence
  B2M-targeting guide sequence portion
ugcugaaagacaagucugaaug

SEQ ID NO: 1450 - RNA, 22 nt, Artificial Sequence
  B2M-targeting guide sequence portion
uggaaagucaaauuuccugaau

SEQ ID NO: 1451 - RNA, 22 nt, Artificial Sequence
  B2M-targeting guide sequence portion
uggagagagaauugaaaaagug

SEQ ID NO: 1452 - RNA, 22 nt, Artificial Sequence
  B2M-targeting guide sequence portion
uggagcauucagacuugucuuu

SEQ ID NO: 1453 - RNA, 22 nt, Artificial Sequence
  B2M-targeting guide sequence portion
uggaugaaacccagacacauag

SEQ ID NO: 1454 - RNA, 22 nt, Artificial Sequence
  B2M-targeting guide sequence portion
uggaugacgugaguaaaccuga

SEQ ID NO: 1455 - RNA, 22 nt, Artificial Sequence
  B2M-targeting guide sequence portion
ugggcugugacaaagucacaug

SEQ ID NO: 1456 - RNA, 22 nt, Artificial Sequence
  B2M-targeting guide sequence portion
ugggggugaauucaguguagua

SEQ ID NO: 1457 - RNA, 22 nt, Artificial Sequence
  B2M-targeting guide sequence portion
uggguuucauccauccgacauu

SEQ ID NO: 1458 - RNA, 22 nt, Artificial Sequence
  B2M-targeting guide sequence portion
uggucuuucuaucucuuguacu

SEQ ID NO: 1459 - RNA, 22 nt, Artificial Sequence
  B2M-targeting guide sequence portion
ugguucacacggcaggcauacu

SEQ ID NO: 1460 - RNA, 22 nt, Artificial Sequence
  B2M-targeting guide sequence portion
uguacuacacugaauucacccc

SEQ ID NO: 1461 - RNA, 22 nt, Artificial Sequence
  B2M-targeting guide sequence portion
uguaguacaagagauagaaaga

SEQ ID NO: 1462 - RNA, 22 nt, Artificial Sequence
  B2M-targeting guide sequence portion
ugucacagcccaagauaguuaa

SEQ ID NO: 1463 - RNA, 22 nt, Artificial Sequence
  B2M-targeting guide sequence portion
ugucggauggaugaaacccaga

SEQ ID NO: 1464 - RNA, 22 nt, Artificial Sequence
  B2M-targeting guide sequence portion
ugucuggguuucauccauccga

SEQ ID NO: 1465 - RNA, 22 nt, Artificial Sequence
  B2M-targeting guide sequence portion
ugucuuucagcaaggacugguc

SEQ ID NO: 1466 - RNA, 22 nt, Artificial Sequence
  B2M-targeting guide sequence portion
ugugaaccaugugacuuuguca

SEQ ID NO: 1467 - RNA, 22 nt, Artificial Sequence
  B2M-targeting guide sequence portion
ugugacaaagucacaugguuca

SEQ ID NO: 1468 - RNA, 22 nt, Artificial Sequence
  B2M-targeting guide sequence portion
ugugacuuugucacagcccaag

SEQ ID NO: 1469 - RNA, 22 nt, Artificial Sequence
  B2M-targeting guide sequence portion
ugugucuggguuucauccaucc

SEQ ID NO: 1470 - RNA, 22 nt, Artificial Sequence
  B2M-targeting guide sequence portion
uuaacuaucuugggcugugaca

SEQ ID NO: 1471 - RNA, 22 nt, Artificial Sequence
  B2M-targeting guide sequence portion
uuaccccacuuaacuaucuugg

SEQ ID NO: 1472 - RNA, 22 nt, Artificial Sequence
  B2M-targeting guide sequence portion
uuacucacgucauccagcagag

SEQ ID NO: 1473 - RNA, 22 nt, Artificial Sequence
  B2M-targeting guide sequence portion
uuacugaagaauggagagagaa

SEQ ID NO: 1474 - RNA, 22 nt, Artificial Sequence
  B2M-targeting guide sequence portion
uucaaugucggauggaugaaac

SEQ ID NO: 1475 - RNA, 22 nt, Artificial Sequence
  B2M-targeting guide sequence portion
uucaauucucucuccauucuuc

SEQ ID NO: 1476 - RNA, 22 nt, Artificial Sequence
  B2M-targeting guide sequence portion
uucacacggcaggcauacucau

SEQ ID NO: 1477 - RNA, 22 nt, Artificial Sequence
  B2M-targeting guide sequence portion
uucacccccacugaaaaagaug

SEQ ID NO: 1478 - RNA, 22 nt, Artificial Sequence
  B2M-targeting guide sequence portion
uucagacuugucuuucagcaag

SEQ ID NO: 1479 - RNA, 22 nt, Artificial Sequence
  B2M-targeting guide sequence portion
uucagcaaggacuggucuuucu

SEQ ID NO: 1480 - RNA, 22 nt, Artificial Sequence
  B2M-targeting guide sequence portion
uucaggaaauuugacuuuccau

SEQ ID NO: 1481 - RNA, 22 nt, Artificial Sequence
  B2M-targeting guide sequence portion
uucagguuuacucacgucaucc

SEQ ID NO: 1482 - RNA, 22 nt, Artificial Sequence
  B2M-targeting guide sequence portion
uucaguaagucaacuucaaugu

SEQ ID NO: 1483 - RNA, 22 nt, Artificial Sequence
  B2M-targeting guide sequence portion
uucagugggggugaauucagug

SEQ ID NO: 1484 - RNA, 22 nt, Artificial Sequence
  B2M-targeting guide sequence portion
uucaguguaguacaagagauag

SEQ ID NO: 1485 - RNA, 22 nt, Artificial Sequence
  B2M-targeting guide sequence portion
uucauccauccgacauugaagu

SEQ ID NO: 1486 - RNA, 22 nt, Artificial Sequence
  B2M-targeting guide sequence portion
uuccauucucugcuggaugacg

SEQ ID NO: 1487 - RNA, 22 nt, Artificial Sequence
  B2M-targeting guide sequence portion
uuccucagguacuccaaagauu

SEQ ID NO: 1488 - RNA, 22 nt, Artificial Sequence
  B2M-targeting guide sequence portion
uuccugaauugcuaugugucug

SEQ ID NO: 1489 - RNA, 22 nt, Artificial Sequence
  B2M-targeting guide sequence portion
uucuaucucuuguacuacacug

SEQ ID NO: 1490 - RNA, 22 nt, Artificial Sequence
  B2M-targeting guide sequence portion
uucucucuccauucuucaguaa

SEQ ID NO: 1491 - RNA, 22 nt, Artificial Sequence
  B2M-targeting guide sequence portion
uucucugcuggaugacgugagu

SEQ ID NO: 1492 - RNA, 22 nt, Artificial Sequence
  B2M-targeting guide sequence portion
uucuucaguaagucaacuucaa

SEQ ID NO: 1493 - RNA, 22 nt, Artificial Sequence
  B2M-targeting guide sequence portion
uugaaaaaguggagcauucaga

SEQ ID NO: 1494 - RNA, 22 nt, Artificial Sequence
  B2M-targeting guide sequence portion
uugaaguugacuuacugaagaa

SEQ ID NO: 1495 - RNA, 22 nt, Artificial Sequence
  B2M-targeting guide sequence portion
uugacuuacugaagaauggaga

SEQ ID NO: 1496 - RNA, 22 nt, Artificial Sequence
  B2M-targeting guide sequence portion
uugacuuuccauucucugcugg

SEQ ID NO: 1497 - RNA, 22 nt, Artificial Sequence
  B2M-targeting guide sequence portion
uugcuaugugucuggguuucau

SEQ ID NO: 1498 - RNA, 22 nt, Artificial Sequence
  B2M-targeting guide sequence portion
uugcugaaagacaagucugaau

SEQ ID NO: 1499 - RNA, 22 nt, Artificial Sequence
  B2M-targeting guide sequence portion
uugggcugugacaaagucacau

SEQ ID NO: 1500 - RNA, 22 nt, Artificial Sequence
  B2M-targeting guide sequence portion
uuguacuacacugaauucaccc

SEQ ID NO: 1501 - RNA, 22 nt, Artificial Sequence
  B2M-targeting guide sequence portion
uugucacagcccaagauaguua

SEQ ID NO: 1502 - RNA, 22 nt, Artificial Sequence
  B2M-targeting guide sequence portion
uugucuuucagcaaggacuggu

SEQ ID NO: 1503 - RNA, 22 nt, Artificial Sequence
  B2M-targeting guide sequence portion
uuuacucacgucauccagcaga

SEQ ID NO: 1504 - RNA, 22 nt, Artificial Sequence
  B2M-targeting guide sequence portion
uuucaauucucucuccauucuu

SEQ ID NO: 1505 - RNA, 22 nt, Artificial Sequence
  B2M-targeting guide sequence portion
uuucagcaaggacuggucuuuc

SEQ ID NO: 1506 - RNA, 22 nt, Artificial Sequence
  B2M-targeting guide sequence portion
uuucagugggggugaauucagu

SEQ ID NO: 1507 - RNA, 22 nt, Artificial Sequence
  B2M-targeting guide sequence portion
uuucauccauccgacauugaag

SEQ ID NO: 1508 - RNA, 22 nt, Artificial Sequence
  B2M-targeting guide sequence portion
uuuccauucucugcuggaugac

SEQ ID NO: 1509 - RNA, 22 nt, Artificial Sequence
  B2M-targeting guide sequence portion
uuuccugaauugcuaugugucu

SEQ ID NO: 1510 - RNA, 22 nt, Artificial Sequence
  B2M-targeting guide sequence portion
uuucuaucucuuguacuacacu

SEQ ID NO: 1511 - RNA, 22 nt, Artificial Sequence
  B2M-targeting guide sequence portion
uuugacuuuccauucucugcug

SEQ ID NO: 1512 - RNA, 22 nt, Artificial Sequence
  B2M-targeting guide sequence portion
uuugucacagcccaagauaguu

SEQ ID NO: 1513 - RNA, 22 nt, Artificial Sequence
  B2M-targeting guide sequence portion
uuuucaauucucucuccauucu

SEQ ID NO: 1514 - RNA, 22 nt, Artificial Sequence
  B2M-targeting guide sequence portion
uuuucagugggggugaauucag

SEQ ID NO: 1515 - RNA, 22 nt, Artificial Sequence
  B2M-targeting guide sequence portion
uuuuucaauucucucuccauuc

SEQ ID NO: 1516 - RNA, 22 nt, Artificial Sequence
  B2M-targeting guide sequence portion
uuuuucagugggggugaauuca

SEQ ID NO: 1517 - RNA, 20 nt, Artificial Sequence
  B2M-targeting guide sequence portion
augcugcuuacaugucucga

SEQ ID NO: 1518 - RNA, 20 nt, Artificial Sequence
  B2M-targeting guide sequence portion
cugcuuacaugucucgaucu

SEQ ID NO: 1519 - RNA, 20 nt, Artificial Sequence
  B2M-targeting guide sequence portion
gcugcuuacaugucucgauc

SEQ ID NO: 1520 - RNA, 20 nt, Artificial Sequence
  B2M-targeting guide sequence portion
ugcugcuuacaugucucgau

SEQ ID NO: 1521 - RNA, 21 nt, Artificial Sequence
  B2M-targeting guide sequence portion
gcugcuuacaugucucgaucu

SEQ ID NO: 1522 - RNA, 21 nt, Artificial Sequence
  B2M-targeting guide sequence portion
ugcugcuuacaugucucgauc

SEQ ID NO: 1523 - RNA, 21 nt, Artificial Sequence
  B2M-targeting guide sequence portion
uucauagaucgagacauguaa

SEQ ID NO: 1524 - RNA, 21 nt, Artificial Sequence
  B2M-targeting guide sequence portion
uuucauagaucgagacaugua

SEQ ID NO: 1525 - RNA, 22 nt, Artificial Sequence
  B2M-targeting guide sequence portion
augcugcuuacaugucucgauc

SEQ ID NO: 1526 - RNA, 22 nt, Artificial Sequence
  B2M-targeting guide sequence portion
ugcugcuuacaugucucgaucu

SEQ ID NO: 1527 - RNA, 22 nt, Artificial Sequence
  B2M-targeting guide sequence portion
uucauagaucgagacauguaag

SEQ ID NO: 1528 - RNA, 22 nt, Artificial Sequence
  B2M-targeting guide sequence portion
uuucauagaucgagacauguaa

SEQ ID NO: 1529 - RNA, 21 nt, Artificial Sequence
  B2M-targeting guide sequence portion
gcauacucaucuuuuucagug

SEQ ID NO: 1530 - RNA, 21 nt, Artificial Sequence
  B2M-targeting guide sequence portion
gcgcgagcacagcuaaggcca

SEQ ID NO: 1531 - RNA, 21 nt, Artificial Sequence
  B2M-targeting guide sequence portion
gcucgcgcuacucucucuuuc

SEQ ID NO: 1532 - RNA, 21 nt, Artificial Sequence
  B2M-targeting guide sequence portion
ucacagcccaagauaguuaag

SEQ ID NO: 1533 - RNA, 21 nt, Artificial Sequence
  B2M-targeting guide sequence portion
gacaaagucacaugguucaca

SEQ ID NO: 1534 - RNA, 21 nt, Artificial Sequence
  B2M-targeting guide sequence portion
acgugaguaaaccugaaucuu

SEQ ID NO: 1535 - RNA, 22 nt, Artificial Sequence
  B2M-targeting guide sequence portion
aagaauguaagacuuaccccac

SEQ ID NO: 1536 - RNA, 22 nt, Artificial Sequence
  B2M-targeting guide sequence portion
ucagcagcuuacaaaagaaugu

SEQ ID NO: 1537 - RNA, 22 nt, Artificial Sequence
  B2M-targeting guide sequence portion
cgucgcgcuggcgggcauuccu

SEQ ID NO: 1538 - RNA, 22 nt, Artificial Sequence
  B2M-targeting guide sequence portion
gcuggcgggcauuccugaagcu

SEQ ID NO: 1539 - RNA, 20 nt, Artificial Sequence
  Example RNA guide sequence portion
aaaaaaauguacuugguucc

SEQ ID NO: 1540 - RNA, 17 nt, Artificial Sequence
  17 nucleotide guide sequence 1
aaaauguacuugguucc

SEQ ID NO: 1541 - RNA, 17 nt, Artificial Sequence
  17 nucleotide guide sequence 2
aaaaauguacuugguuc

SEQ ID NO: 1542 - RNA, 17 nt, Artificial Sequence
  17 nucleotide guide sequence 3
aaaaaauguacuugguu

SEQ ID NO: 1543 - RNA, 17 nt, Artificial Sequence
  17 nucleotide guide sequence 4
aaaaaaauguacuuggu

SEQ ID NO: 1544 - RNA, 21 nt, Artificial Sequence
  TRAC-targeting gRNA32 guide sequence portion
gccacuuucaggaggaggauu

SEQ ID NO: 1545 - protein, 1348 aa, Artificial Sequence
  OMNI-103 amino acid sequence
MSIKSDYFLGLDIGTDSIGWAVTDPEYHILRRKGKALWGIRLFDAANTAAERRTFRTSRRRIQRRRQRIRLLQELFAEEMVKLDPGFFQRLSDSAFWQEDKQEQQIYSLFTCENYTDVDYYREYPTIYHLRSALIQEKKEFDLRLLYLALHHLMKHRGHFLFNGSINNVTSFHTTFQTFADCLYDEFDIELECDSEDRFAEILKDKHARKTGKCSELEIICHIEKSNKQLKELFKLITGMKASLSVVFGDDELAEIEHNKISFSESSYDEVRLALEDEIQERTGILDIFHAVYSWAILADILEGGEYEGNSYLSVAKVSTYKKHGDDLRLLRTLVREYCPDHYKSFFSVSGKENYCAYAGTLKKNGKKQPIKRCSQEDFYKALKKLLNQMPTEQPEVKDIFIEIENGTFLPLQVSKDNGVIPYQVNKMELEKILQNAEEYLPFLKNIDEECGKTVSKKIIDLFEFRIPYYVGPLNTAKGENCWMVRKEAGRIYPWNFDEKVDRDQSAEKFIRRMTNQCTYLIHEDVVPKNSLLYSEFMVLNELNNVKIRSEKLPVELKQAIVLDLFKKQKQITGKKLLNYLNANGYDVKKEDLSGFDGNFKSSLSSYLTLKKVFGEELDKYSVQQMAEDIILWITLYGDDQKMLRRVIRKHYEQQLSEEQILSLSKLKFQGWGRLSRRLLSEMEGVDCETGECMTVMQGLRNTQNNLMQLLSQQFSFMELIEEENGNYYVDEITYDNLVKDMVISPSVKRAVWQTVQIVEEIKGVMGCQPKKIFVEMARSDEEKKRTVSRKDRLLEAYDAIKDEARQWQEELQKYSDGDFKAIKLYLYYTQMGQCMYTGRKIDLSQLNDATVWDRDHIYPQSKTKDDSLDNLVLVDRSVNAKKSDGMLSPEIQQRMRATWKYLKEKKLISEKKYERLTRVSPLTDEELAGFINRQLVETRQSSKAVATLLKRVYDEAEIVYVKAEAVSNFRRDNLDYIKVRDLNDYHHAKDAYQNIVVGNVFHEKFTSNPLRWLKNNPNTKYSLNQMFNFDLEKNGVVIWKRGKAGSIKCVEETLKRNDILFTRYAFCNKGGFFNQMLTAAPEDKTKAKGLVPIKKGMETWKYGGYTSVTPSHFMLVASKDKKGKEIRTIETVPLYRWKEFKENPDALLQYCREFYGLKEPKVLIPCIKKNARLVVNGFPMHLKGSTGKQLILQGAVQLCLNNENIKYLKKVTKYLEYNAQRRDKRTLLEVREVTGINKEENIQLYDVFVDKLSNTIYQYRPANPKDNLIKGREKFIELGLAEQCVVLGEVLHLFQCKPLTSDLTLIGGSPNTGTIKITKTISNCNVVKLLSQSIAGVKVREINLLII

SEQ ID NO: 1546 - protein, 1097 aa, Artificial Sequence
  OMNI-124 amino acid sequence
MGKIIGLDIGIASVGWAVLDKDSYKIVEAGSNIFPAAEASKNQERREFRGLRRMTRRRRTRLDDFKKLWKNYYKNEYSVTPNNVTELKVKGLSEQLSELELYTVLYSYLKHRGISYLEDAVDEGKTGSDYLKGLQTNQKEMNTKFPSQIQYERLEKYGCFRGENKVSDDEVLSNVFTTSAYKKEIEELISVQDNLGNELDKDFIKKYFEIFSRKRKYYEGPGNELSRTDYGIYTTKIDENGNYITDKNLFEKLIGKCSVYPEEMRASAASYTAQEFNALNDLNNLIVNGRKLTKEEKISIIECYKTEKTINVRKIIKNVIGENIDQLSGYRMDKNDKELYHTFEVYNKLRKAFEEAGISFDFSREDFDEIGRILTLNIEREGIEDAIRISNLVLSSDAVDVLISVRKKNASLFSKWHSFSLKIMNELIPEMYEQPKNQMELLSDMKSIKSKMDEFKGLEDIPVETIIEDIYNPVVKRSIRITIKIINALKKKYSDIEKIVIEMPRDKNSDEEKERIKKFQKDQENELPNIIKRVKDEYGITLTPELIARKKGLAFKLKLWNEQNGRCPYSGRMIDIESLINDGDLFEVDHIIPLSISFDDSRSNKTLVYRVENQEKKNNTPFMYLSRLNRQWNYDKFAAFIKELYGSSGKDRKKFRKKMNNFLFTEDINKIDVLKGFINRNINDTRYASRVVLNSLQGYFRANDNNVKVSVINGSFTHQMRVNLSLGKDRDESFAHHAVDAMLIALSQMGYDAFRARQKDIVDFETGEIINGSAWENQDEMYKTVLYQEKWKNIKDNIIEAEPNIKYWHKVDKKCNRGLCNQTIYSTRKCDDKIWKVDKLNIRDKDDVKKLIYMIQNGKEDKILMAQNDPKTFANLKYILDEYSDCENPFVSYEKETGDLVRKYSKKHNGPKITYLKYMVEEVNSCIDISHKYGHEKGSKKVFLAQLVPFRTDVYYNNNSGKYYLIGIKQSDIKCKGNQYIIDEEKYTDNLIQNGLLKEGQKIEDLEKLGYLFKFSIFKNDTIEFEKKGELIKCRFLSQNSASKNRIEVKPIGKKDYESRNLPTLSTTTVINKYVKDILGNEKQVSGEKFEIIVDSK

SEQ ID NO: 1547 - protein, 1109 aa, Artificial Sequence
  OMNI-127 amino acid sequence
MGKLVLGLDIGITSVGYGVIDIDNNKFVDYGVRLFKEGDSNDNEKRRSYRSGRRLRRRKKNRLDDMKSLLLSAGILTKDYKPLNNIYEIRVKGLTEKLTNNELAAALLHITKNRGTTVEALETADADKEAAGTKDVLAKNAKDLEQFEYVCKVQLYRLEKYGNIRGTENNFNTSDYVKEARAILKCQDIDVELSESIVNIISRRREYYEGPGSEKSPTKYGRWIEKGAEPIDLIEKMRGKCSIYHDELRAPKSSYSAELFNLLNDLNNLSINGEKLTQNEKEIVIEEVNNNGKITIDKLKKLLNINDSKDVVEGFRINKSNELLLTNMAMYDQFKKIFDKHKEDAYQRDKYIIDQIAEINTAKKGIKERCDAIEKQFPNFDYNLIRDLAEMKKMTQYHSLSLKAIREINKEMIKSPYNQMQAIHELGLQHYRKSYKGMKKIQVDEEAILSPVVKRAQRETFKVINRLREIYGEFESIVVETTRDKNSKEKKERINKTQKYYEDRNKKVDELLKERGYNPDTINNKTKMKVKLYLDQNGKSAYTMTPLKLNLIITNPHAYEIDHIIPLSISQDDSFNNKVLITHEENQVKGNKTPIYTYLNNQFKNCDINKYKESVKNNHNFSRKKKFYMLYEKDITKYSNMKEFIARNLVDTSYASRVVLNTLTSYFRDNDISTNVHTIRGSATSLYRKRIGLKKDRDKDFLHHAIDALIVASIKKQSIFRKYLIKYNLSELYNEKTGELIKVEEDSKVIDPVFIEFISNLKTIYEQSYQYYNGYISRNKMKFKPIKISHKVDTKPNRKISDETIYSTRNVNGVERVVKKVKDIYDPKFDKLTNDIINNQYHNKWIMAEKDEQTFEIIKSIVMAHFNTYKEDKDIYKSKSKKGVTTWSLKGSNNPLSQFKEEHGKIRKYSKKGNGPEITSMKYYEDELGNHVNITKKYNVDNKNVVLLQISPYRTDFYVSPKGKYKFVTIRYCNVFYKESNKKYLIDKKWYEEEKKKKKIDDSYQFVCSLHHDEILGVKLEDGKPYIYDESNENDEGLKRYHKGQYEILKFTATNDDKKNIIEIKPTYTYCQKQLKRSCGTFIKLTKFATDVLGNMYEVKDNVLKLEFD

SEQ ID NO: 1548 - protein, 1215 aa, Artificial Sequence
  OMNI-218 amino acid sequence
MSKKRILGLDTGTNSLGWAVVDQEENGQYTLIDKGVVIFQEGVKIEKGVESSKAAERTEHRALRRHYFRRRLRKIEVLRVLVKYNLCPTITEEDLNLWKLHKTYPRKDEFMVWQHTNDNEDINPYHCRYTCLNENLDLDRQQDRYILGRALYHMAQRRGFLSNRLDATEEDENGAVKSSIANLNKEIEENGCKYLGEYFYKLYKEKGNTVRIRTRYTDREQHYKKEFFAICEKQNLPKEMVEALTRALYFQRPLKSQRQNVGKCTFEKGKPRCSDSHPTYEIYRMLSFLNNVKIQGPWDEDLRPLNEEELKQIDKLFYRKSKPNFDFEEIAKKIAGKGKYQWKGDTKTDLPYKFNYRMSQGVPGCPTIAQLKEIFGENWKENIAETYTLNTKKDGTHKSADEMVDDIWNVLFFFDSKDKVKEYGIKHLQLDEDQADKFSKIRLSRNYASLSLKAIRYILPFLHKGMIYSDAVFFAKIPVIIGQSLWQKRQHEIMEDLSYAIHMAMKEKQRIDDAVKDYLQNNFDLKPGAADMLYHPSMIETYPDAQPDKNGVVLLGSPRTNAIKNPMAMRSLHEIRKVINTLIKEGKVAPDTEIHIEYARELNNANMRAAIHDLQREKDKKHKEYSENIRKLYKEATGKDIEATETDILKFQLWEEQGHICLYTGDEIGIADFIGANPSFDIEHTIPRSVGGDSTQMNMTLCNTHYNRFVKKAYIPTQLADHEFIMTRIEPWKEKIEDLTKQINQIRTSNIWDKAAKDAKIRKKNRLLEELKYWKGKYDRFTMTEVPEGFARRQGAGIGLISKYAALYLKSYFHDTLHPEHRQVYSIKGTITSDFRKMWGIQEEDEKKSRDSHTHHCIDAIVIACIGKNEFNMMGQYYHVQEKFEKGEGEKPEFKKPWPTFTQDLKKIKEELVVVHDTPDNMPKKATKTIEISKGRKVVAKGDCARGSLHQDTYYGAIERDGNIKYVVRKPLSSFTSIKDLDNIVDDAVRQAILNAIEGKDFKKALAEPIYMNKDKGILIKKVRCYTPSITKPLHIRTHRDISQKEYKQQFHVALDSNYCMAIYEGEVKGKTKRTYEIINMLDAAAYFKKSSNLAKQHPIAPENKNELKYRCSVHTGTQVLLLQSDGENINLENKDDIARRLYHVAVMRSDGRIVLRYNQDARDATTLKKERKSGAYKVGEQYRSEIMLSLSDFHALVEGYDFTITPLGEIKLKH

SEQ ID NO: 1549 - protein, 1350 aa, Artificial Sequence
  OMNI-237 amino acid sequence
MGNQSGQEYYLGLDIGTDSAGWAVTDPEYRILRRKGKSLWGVRLFESANTAAERRAFRTNRRRLQRRRQRILLLQELFAEEMEKVDAGFFQRLNDSACWAEEKEEPQIYSLFGGKDYTDVDYYREYPTIYHLRSALLRGKQEYDLRLVYLALHHLMKHRGHFLFQGNIESVTSFQTTFQNFQDCLMDEFGLELECDSEERFSELLKDRKIGRMEKCRQLELLCHVQKTDKQKKEIFKLITGMKAGLSVIFADEELAQIDYNKISFAESGYEEARLALEEEIQERTGILDVFHAVYSWAVLADILAGGEYEGRPCLCVAKVNTYEKHRQDLRLLKKLVREYCPEDYKSWFSVPVNDNYCAYIGTFRKNGKRCETKRCNQETFYKALKKLLGKMSADLPEAADILKETENGTFLPLQVSKDNGVIPYQVHKMELERILDHAEKYLPFLLEKDPECGKTVREKIVALFEFRIPYYVGPLNTAAGENCWMERKEEGAVRPWNFDQKVDRDKSAERFIRRMTNQCTYLIHETVLPKHSLLYSEFMVLNELNNVKIRDEKLPVELKQGIVQKLFKTRKQVTGRKLLDYLTANGYDVKKDELSGFDGNFKASLTSYHTLKKIFGKELEKSSVQQMAEDIILWITLYGEDQKMLCRVIRQHYGEILKEEQLKELSRLKFQGWGRLSRRFLNELEGVDCTTGECMTIMQGLRNTQNNLMQLLSQQYTFARMIDDENGGYHVDEITYDNLVKDIVASPSIKRAVWQTIQIVEELKNVMGCEPKKVFVEMARGEEEKRRTVSRKDKLLEAYAAIKDEARDWQEELQKYSEGDLKAIKLYLYYTQMGQCMYTGKKIELSQLNNATIWDRDHIYPQSKTKDDSLDNLVLVDRRVNAKKSDGMISPEIQQKMRGTWKYLKEKGLISEKKYERLTRTTPLTEEELAGFINRQLVETRQSSKVVATLLKRIYEEAEIVYVKAKAVSDFRKEHLKYVKVRDLNDYHHAKDAYQNIVVGNVYHTKFTGNPLRWLKEHPERKYSLNQMFNYDLEVHGKPVWKRGKDGSIRCVSETLKRNDILFTRYAFCNKGGLFNQTIMKAPEDKAKAKGLVPIKKGKNPEKYGGFTSVTPSHFMLVASEGKKGKEIRTIETVPLYRKKEFEREPELLLQYCREFYGLKNPRILISCIKKNARLVINGFPMHLKGSTGKQLILQGAVQLCLDDEKVVYLKKVTNYLEENARRKDKRTLLEVKEITGITSEANINLYDLFIKKLSSTIYQYRPANPKDNLLKGREKFLNLGLAEQCVVLGEILHLFQCKPLSANLILIGAAGNTGVTKIGKEISRCNSAKLVSQSVTGIYEQVVDLLTI

SEQ ID NO: 1550 - protein, 1078 aa, Artificial Sequence
  OMNI-291 amino acid sequence
MQWPYGVGLDIGVASIGWATVALDEKAQPCGLIRLGSRVFKRAEQPKTGESLAAPRREARGMRRRLRRRALRKQDFYQLLEKNHLPDKEELSRLFAAGGLEDIYALRTRALDEAVTPQEFARILLHLMQRRGFKSNRLAGKSKKDGVLLQAVTQNQLRMQENGYRTVGEMLYKDPLFAACKRNKGENYLSTVSREEIANEAQLLFEAQREKGRDWATPELEAEYLSILLRQRSFDQGPGGNSPYKGGWAERVGPCTLLPEEKRAFKNTYSFERFTLLQKINNIRIVESGQTQALSDEERQKVMDLAYRVETVPYTLIRKELQWPQTIRFADVRYDVDAAPEAAEKKCKLPPMKGYHALRKALGTRQAELPLWDDVITILASHRDEGVRKKLLEEKGLTAEQVEAVLGLEFSGVGHISLKACQMLEPWLLQGLKYDQACSKAGLDFRGHSNIKKTPKLPASAPELKDITSPVVRRAVSQTIKLVNAIIREMEQPPVWIHIELARELSRTFEERRRDQKSMEDNARQNECLMQEIRDTFHKLNPTGQDLIKYRLWKEQDGICAYSQQSIAVERLFETGYAEVDHIVPYSISFDDSRSNKVLVMAAENHQKGNRLPLQYLQGERREKFIVWTKSQVRNYRKQKNLLRETVSEEEQNGFKQRNLQDTQYMARFLLNYIRDHLAFADHPAAGKQRVMALAGAVTSHLRKRWGLTKVRADGDLHHALDAAVIACATKGMVQQISGYYHRIEGEYQQSSDGTYSVHTRTGEVFPAPWPHFRDELIQRLSQKPQENLMQWNPVFYSNFDVESIRPVFVSRMPQHKVTGAAHKETIKSPKALQDGLLIVKHPLTDLKLNKKTGEIEGYYQPSSDKLLYEALRARLLEFGGDGKKAFTEPFYKPKSDGTPGPLVRKVKLCEKASLCVPVHGGNGAADNDSMVRVDVFFVPGDGYYMVPIYVSDTKKKELPNRAVVAHKEFSAWKVMEEKNFLFSLYADDLIEVARKKVMPFTIVNSGSTLPPSYETTHEMVYYGGLDISSGNIKVTIHDNSYSLRGLGIKTLNTLQKYEVDILGNVREVKKEKRQSFR

SEQ ID NO: 1551 - protein, 1097 aa, Artificial Sequence
  OMNI-308 amino acid sequence
MAYAIGLDIGIASVGYAVVALDYEENPWGIINMGSRIFDAAENPKDGASLAAPRREARTGRRRLRRRKHRIERIKNLFLNTQLLTKEQLEKLYEGVLSDVYELRVKGLDEKLTAEEVARVLLHLAHRRGFKSNRKAEKTDKENGALLTAVSENQKRMKDKQYRTIGEMFHKDELFAEYKRNKGGTYLTTVAREMIADEAKYIIATQRSFGNKTLTIEFEESYLDILLSQRNFDEGPGGNSPYGGDQIERMIGNCTFEKSEKRCAKSCYSFEYFNLLQKINHLRLLVDGVKVELDGSQKSKVINLAKTKADLKYSQIRKELGLGSQVLFNALVYNSDVEEVEKKSKFNFMPAYHQIRKELDTVVKGRIEAIDIDTLDEIGRILSTYKSDAKRIEAFSNLNITSKDMEALLNINGLRKFGHLSLKALKKITPFLEQGMIYNEACTAAGYEFRGHSGYEKTFLLPSQTEEMENVTSPVARRSISQSIKVINAIIREQGESPLYINVELAREMAKDFDERKKIKKETDENQARNERLIEKIRNEFHKQNPTGLDLVKLKLWEEQDGVSPYSQKQIVIERLFEPGYVDVDHIVPYSISFDDTMKNKVLVFASENREKGKNLPVEYLARKKGQKAVDDFKVWVTSNVRNYKKRLNLLKSEVTEEDLNKFKERNLQDTKTISRFMYNYISDHLIFAPSETGKKKRVTAVNGAITSYLRKRWGINKVRADGDKHHAVDALVIACTTDKLIKDLSYHSRYLEMEYEAGEKESLWVNRNTGEIVKRFPYPWEDFRAELMARLSDAPEVALRKLQLLYYQDKDLSKIKPIFVSRMPRRKITGAAHKATVRGARYVDKGIVISKKSLSELKLNKGGEIENYFAPESDRLLYEALKEQLKKFNGNAKKAFEQGFYKPKADGTPGPKVNKVKVYDKSTLNVAVQQGTGAADNDSMVRVDVYKVEGDGYYLVPIYVADTLKKELPNKAIVQGKTYEQWKEMSPEDFVFSLYPNDLIKITHKKEINFSRVSEDSTLADSYKTKEEFVYYKKTSISTASISIINNDNTYTIPSLGVKTLASLEKYQVDILGNYTKVGKEKLQTFAAYQQAKKKP

SEQ ID NO: 1552 - DNA, 4047 bp, Artificial Sequence
  OMNI-103 DNA sequence
atgagcataaaaagtgattattttttaggacttgatattggtacggattctattggatgggcggtaaccgacccagaatatcacatattgagacgaaaaggtaaagcattatggggaataagattatttgatgcggccaatacagcggcagaacggcgaacatttaggacaagccgaagaaggattcagagaagacgacagagaattcggttattgcaagaattatttgcagaagaaatggtaaaattagacccaggattttttcagaggttgtcagacagcgcattttggcaggaggataagcaagagcagcaaatttattcactttttacttgtgaaaattatacagatgttgattattacagagaatatcctactatttatcatttgagaagtgcattgattcaggaaaagaaggaatttgatcttcgtcttctatatcttgctcttcaccatttgatgaagcacaggggacatttcctgtttaatgggagtattaataatgtgacgtcatttcatacgacgtttcagacgtttgcagattgtctttatgatgagtttgatatagaactggaatgtgattccgaagatagatttgcagaaattttaaaggataaacatgccagaaaaacaggaaaatgttctgaattagagataatctgtcatatagaaaaatcaaataagcagctaaaagaactttttaaattaattacaggaatgaaagctagtttgagtgttgtgtttggtgatgatgagttagcggaaatagaacataataagattagtttttcagagagtagttatgatgaagtacgtcttgcattggaggatgagattcaggagaggactggtatactggatatctttcatgcagtttatagttgggcgattctcgcggatattttagaaggcggagaatatgaggggaattcttatctaagcgttgcgaaggtaagcacttataaaaagcatggtgatgatttgcggttgctaagaacactggttcgggaatattgtcctgatcattacaaatctttcttttccgtatcagggaaggagaattattgtgcatatgcgggtactttaaaaaagaatggaaaaaaacagccgattaaacgttgcagccaggaagatttttataaagcgttaaagaaattgctgaatcagatgccgacagaacaaccagaagtgaaagacatcttcatcgaaattgaaaatggtacttttttgccgttgcaggtaagtaaggataatggagtgataccttatcaggtaaataagatggaattagaaaaaatcctgcagaatgcagaggaatatttgccatttctaaaaaatatagatgaagaatgtggaaaaacggttagtaagaagattatagatctctttgagtttagaataccatattatgtagggccgcttaataccgctaaaggagaaaactgttggatggtcagaaaagaagcggggagaatatatccgtggaattttgatgaaaaggtagacagagatcaatcagcagaaaaatttatccgtagaatgacgaatcagtgtacatatttaatacatgaggatgttgtacctaaaaattctttgctttattcggagtttatggtgcttaatgaattaaataatgtaaagatccggtctgagaagctgccggtggagttaaaacaggcaatagtattggatttgtttaagaaacaaaagcagataacaggaaaaaaacttcttaattacttgaatgcaaatggatatgatgtaaaaaaagaagatttgtcagggtttgacggaaactttaaatcatctctgtcatcatatcttactttgaaaaaagtatttggtgaagaattagataaatatagtgtgcagcagatggcagaggatattatcttgtggatcactctgtatggagatgatcagaagatgttgcgcagggtaattcgaaaacattatgaacagcaattgagtgaagaacagattctttccttatcgaaattgaaattccaaggctggggaagattatccagacgacttttgagtgaaatggaaggcgttgattgtgagactggtgagtgtatgacggtcatgcaaggacttcgtaatactcagaataatctgatgcagcttctaagtcagcagttttcatttatggaattgattgaggaagaaaatgggaattattatgtagatgagattacatacgataatcttgtgaaagatatggttatatctccgtcagtgaagagagcagtctggcagacagttcagattgtggaggagattaagggggtaatgggctgtcagcctaagaagatatttgtcgagatggcgcgaagcgatgaagagaaaaagcgtactgtatctaggaaagacaggttattagaagcatatgatgcgatcaaggatgaggctcgtcaatggcaggaagagttgcaaaagtattcagatggtgattttaaggctattaaactttatctgtattatacgcagatggggcaatgtatgtatactggaagaaagatagatctgtcacaattaaatgatgcgacggtatgggacagagatcatatatatccacagtccaaaacaaaagatgatagtctggataatctggtattggtagaccggagcgtgaacgctaagaaaagtgatgggatgctatcacctgagattcagcagagaatgcgggctacttggaaatacttaaaagagaaaaagttgatttcagagaagaaatatgagcgtttgactagggtctcaccacttacagatgaggaattggcaggttttattaatcgacagttagttgaaacacgtcagtcttcgaaagcagtagcaacacttttgaaacgagtatatgatgaagcggagattgtctatgtaaaagcggaagctgtttcaaattttagaagagataatttggattatattaaggtgcgtgatctgaatgattatcatcatgctaaagatgcatatcagaatattgtagtggggaatgtttttcatgagaaatttaccagcaatccgcttcgttggctgaaaaacaatcctaatacgaaatatagtttaaatcagatgtttaactttgatttagagaaaaatggggtggtaatatggaaaagggggaaggctggaagtattaaatgtgttgaagaaacattgaaaagaaatgatattctttttacacgatatgctttttgtaataaaggtggtttttttaaccagatgttaacggcagctccagaagataaaacgaaagcaaagggacttgtaccaataaaaaaaggtatggaaacatggaaatacgggggatatacatcagtaactccgtcacattttatgttggttgcttcgaaagataagaaaggaaaggagataagaacgattgagacagttccgttgtataggtggaaagagttcaaagaaaatccagatgcattactccaatattgtagagagttctatggtttgaaagagcccaaggtgttgataccatgcatcaagaagaatgccagattagtcgttaatggatttccaatgcatttgaaagggagtacaggaaaacaattgattttgcagggagcggttcaattatgtctgaataatgaaaatataaagtatttgaaaaaagtcacaaaatatttggaatataatgcacagagaagagataaaagaacactgctggaagtaagagaggttacaggaattaacaaagaggaaaatatacagttatatgatgtgtttgttgataaattgagtaacacaatatatcagtatcgtccggccaatccaaaggacaatcttataaaaggaagagagaagtttatagaattagggttggcagaacaatgtgttgtgttaggtgaagtattgcatttgttccaatgtaaaccactcacgtctgatttgactttgattggaggttcaccgaatacagggacaataaaaataacaaagacaattagtaattgtaatgttgtaaagttgttaagccaatctattgcaggtgttaaagtgagagaaattaatttgttaataatatga

SEQ ID NO: 1553 - DNA, 3294 bp, Artificial Sequence
  OMNI-124 DNA sequence
atgggaaagattataggactagatattgggattgcatcagttggttgggcagtgttagataaggatagttataagattgttgaggctgggtctaatatatttccggctgcggaagcaagtaagaatcaagaaagacgtgaatttcgtggcttgagaaggatgactagaagacgaagaacccgtttggatgattttaaaaagctatggaagaactattataaaaatgagtattcagtaacacctaataatgttacagagttaaaagtcaagggattatcagaacagctatcagaattagaattatacactgttttatattcgtatttaaagcatcgtggtatatcttatttagaggatgctgttgatgaagggaaaactggaagtgattatctaaaaggacttcaaacaaatcaaaaagagatgaatactaaattcccaagtcagatccaatatgaaagactagagaaatatgggtgttttcgtggagaaaacaaggtgagtgatgatgaggtattgagtaatgtttttaccacatcggcatacaaaaaagaaattgaagaactgattagtgttcaggataatcttgggaatgaattagataaagattttataaaaaagtattttgagatattttcgagaaaaagaaaatattatgaaggtcctggaaatgaattatcaagaacagattatggtatatatacaactaaaattgatgaaaatggcaattatatcactgataaaaacctttttgaaaaattgattggtaaatgttctgtttatcctgaagaaatgagagcatcggctgcatcatatactgctcaagagtttaatgcattgaatgacttaaataatcttatagttaatggcaggaaactaactaaagaggaaaagataagtattattgaatgttataaaactgaaaaaacaataaatgttagaaaaatcatcaaaaatgtgataggagagaatattgatcaattatccggatatagaatggataagaatgataaagaactttatcacacctttgaggtttataataaactgagaaaagcctttgaagaggcgggaatcagctttgatttttcacgtgaggattttgatgaaataggaagaattttaactttaaatattgaaagagaaggtatagaggatgctattagaatcagtaatctggttttatctagtgacgcagttgatgtactaatatctgtaagaaagaaaaatgcttctttattcagtaaatggcattctttttctttgaaaattatgaatgagctcattccggaaatgtatgaacaaccaaagaatcagatggaattattgtctgatatgaagtcaatcaagtccaaaatggatgagtttaaaggcttagaagatattcctgtcgagactattattgaggatatatacaaccctgtagtaaagagatccatcagaatcacaatcaagataataaatgcattaaagaaaaagtattctgatatagaaaaaatcgtaatcgaaatgccaagggataagaattccgatgaagaaaaggagcgaatcaaaaagtttcagaaggatcaagaaaatgaattgcccaatattataaaaagagtaaaagatgagtatgggataacactgacgccggagttgatagccagaaaaaaaggattggcttttaaacttaaattgtggaatgaacagaatggaagatgtccgtattccggacgaatgattgatatagaaagcttgattaatgatggtgatttgtttgaagtggatcatataataccactctctatttcttttgatgacagtagatcaaacaagacgcttgtgtacagagtggaaaatcaggaaaagaaaaacaatacaccatttatgtatttgagtagattaaatcgtcagtggaattatgataaatttgcggcttttattaaagaattgtatggtagttccgggaaagatagaaaaaaattcagaaagaaaatgaataattttttatttacggaagatataaataagattgacgtattaaaaggatttattaatagaaatataaatgacactcgctatgcttcaagagttgtacttaattcattgcaaggatactttagagctaatgataataacgtaaaggtgtcggttataaatggcagtttcactcatcagatgcgtgttaatctatcattagggaaagatagagatgaatcctttgctcatcatgctgtcgatgctatgttaattgctctgtcacaaatggggtatgatgcttttagagcgagacagaaggatatagtagattttgagactggtgaaataattaatggtagtgcctgggaaaatcaggatgaaatgtataagacagtcttatatcaggaaaaatggaagaatataaaggataatatcatagaggcagagcctaatattaagtattggcataaggtcgataaaaagtgtaacaggggactatgtaatcagactatatattcaaccagaaaatgtgatgataagatatggaaggttgataaactgaatattcgagataaggatgatgtaaaaaagctgatctacatgatacaaaatggaaaagaagataaaatacttatggctcaaaatgatccaaagacttttgctaatctgaagtatattttagatgaatatagcgattgtgagaatccgtttgtatcttatgaaaaagaaactggtgaccttgtaagaaagtattcaaagaagcataatggacctaaaataacatatttaaaatatatggttgaggaggttaatagctgtatagatatatcacataagtacgggcatgaaaaaggatcaaaaaaagtctttttggctcaattggttccatttagaacagatgtctattacaataataacagtggtaaatactatctcatagggataaagcagtcagatataaagtgcaagggtaatcagtacattattgatgaagaaaagtacacagataatttgattcaaaatggtcttttaaaggaaggacagaaaatagaagatttagagaaacttggctatttatttaaattttcaatctttaaaaatgatactattgaattcgagaagaagggagaattaatcaagtgtagatttttatctcaaaattcggcatctaagaatagaatagaggtaaaaccaatcggaaaaaaagattatgaaagtagaaatttgcccacgctttcgactacaacagttattaataaatatgtaaaggatatattgggaaatgaaaagcaagtaagtggcgaaaaatttgaaataattgttgattcaaaataa

SEQ ID NO: 1554 - DNA, 3330 bp, Artificial Sequence
  OMNI-127 DNA sequence
atgggaaaattagtgttaggtttagacattggaattacttctgtcgggtatggagtaattgatatagataataataaatttgtagattatggagtaagactttttaaagaaggtgattctaacgacaatgaaaaaagaagatcatatagaagtggaagaagattaagaagaagaaaaaagaatcgtttagatgatatgaaaagtcttcttttgagtgcaggtatacttacaaaagactataaacctcttaataatatttatgaaattagagtaaagggattaacagaaaaattaacaaacaacgaattagccgctgctcttcttcatattacaaaaaatagagggactactgtcgaagctctagaaactgctgatgcagataaagaagcagcaggaaccaaagatgtattagcaaagaacgcaaaagatttagagcaatttgagtatgtatgtaaagtacagttatatcgtttagaaaaatatggaaatattaggggaacagaaaataactttaatacttctgattatgtaaaagaggcaagagcaattttaaagtgtcaagatatagatgtagaactatctgaaagcattgtaaatatcataagcagaaggagagagtattatgaaggacctggtagtgaaaaatctccaacaaaatatggaagatggatagaaaaaggagcagaaccaatcgatcttatagagaaaatgagaggaaaatgttcaatatatcatgatgagcttcgagcaccaaaatcctcatattctgctgaattatttaatttgttaaatgacttgaataacctttcaattaatggtgagaagttaactcaaaatgaaaaagaaatagttatagaagaagtaaataataatggaaaaatcactatagataaattaaagaaattattaaatattaatgatagtaaagatgttgtcgaaggattcagaataaataaaagtaatgagcttttattaacgaatatggcaatgtatgaccagtttaaaaaaatttttgataagcataaagaggatgcttatcaaagggataagtatattattgatcaaattgctgaaattaatacagctaaaaaaggtattaaagaaagatgtgatgcaattgaaaagcaattccctaattttgattataatttaatcagagatttagctgaaatgaaaaaaatgacacagtatcattcattatcattaaaagcaattcgagaaataaataaagagatgattaaaagtccgtataatcaaatgcaggcaatccatgaattaggattgcagcactatagaaaatcttacaaaggaatgaagaaaattcaagttgatgaagaagcaatattatctccagtagttaaaagagcacaaagagaaacattcaaagtaattaacagattaagagaaatatatggtgaatttgaatcaatagttgttgaaacaactagagataaaaatagtaaagaaaaaaaggaaagaattaacaaaactcaaaaatactatgaagataggaacaaaaaagttgatgagttgttaaaagagagaggatataatccagatactataaataataaaacaaagatgaaagttaagctctatcttgatcaaaatggaaagagtgcatatacaatgacacctcttaaattaaatcttattattactaatcctcacgcatatgaaattgatcatattattcctctttcaatttctcaagatgattcatttaataataaagttcttattacacatgaagaaaaccaggttaaaggtaacaaaacaccaatatatacttatttaaataatcagtttaaaaattgtgatattaataaatataaagaatcagtaaaaaataatcataatttttcacgaaagaaaaagttttatatgctctatgaaaaagatattactaagtattcaaatatgaaagaatttatcgcaagaaacttagttgatacaagttatgcaagtagagtagttcttaatacattaacctcatattttagagataatgatatttcaacaaatgttcatacaattagaggatcggctacaagtttatatagaaaaagaattggactaaaaaaagatagagataaagattttcttcatcatgctattgatgctttaattgttgcgtcaataaaaaaacaatctatttttagaaaatatttaattaaatataatcttagtgaattgtataatgaaaaaactggagaattaatcaaagttgaagaagacagtaaagtaattgatccagtatttatagaatttatttccaatttgaaaacaatttatgagcaatcatatcaatattacaatggatatatttctagaaataaaatgaaatttaaaccaattaaaatttcccataaggttgatacaaaaccaaatagaaaaatttctgatgaaacaatctacagtacaagaaatgtaaatggtgtagaaagagttgtaaaaaaagttaaagacatttatgatccaaaatttgataaactaacaaatgatattattaataatcaatatcataataagtggataatggctgaaaaggatgaacagacatttgaaattattaaatcaattgtgatggcacattttaatacttataaagaagataaggacatatataagagtaaatcaaaaaaaggtgttactacttggtctttaaaaggaagtaataatccattatcacagtttaaagaagaacatggaaaaatacgtaaatattctaaaaaaggaaatgggcctgaaattacttctatgaaatattatgaagatgaattaggaaatcatgttaatatcacaaaaaaatataatgtagataataaaaatgtagttttacttcagataagtccatatagaactgatttctatgtttcaccaaaaggaaaatataaatttgtaactataagatactgcaatgttttctataaagaatcaaataaaaaatatttaattgataaaaaatggtatgaagaggagaagaagaaaaagaaaattgatgattcatatcaatttgtatgttctttgcatcatgatgaaatattaggagttaaattagaagatggtaaaccttatatctatgatgagtcgaatgaaaatgatgaaggtttaaaaagataccacaaaggacaatatgaaattttaaagtttacagcgacaaatgatgataaaaaaaatataatagagattaagcctacttatacatattgtcaaaagcaattaaagcgaagttgtggtacgtttattaagcttactaaatttgctactgacgtattaggaaatatgtatgaagtaaaagacaatgtcttgaaattagaatttgactag

SEQ ID NO: 1555 - DNA, 3648 bp, Artificial Sequence
  OMNI-218 DNA sequence
atgagcaaaaaacgtattttaggacttgacacaggcacaaacagtctaggttgggcagttgttgaccaggaagaaaacggtcagtacacattaattgacaaaggcgttgtaatcttccaggaaggagtaaaaattgaaaagggtgtcgaatccagcaaagcagctgaaagaacggagcacagagcacttcgcagacattattttcgcaggcgtttgcgcaagatcgaggtattaagggttttggtaaaatataacctctgccctaccataacggaagaagacctgaatttatggaagttgcacaaaacatatcccaggaaggacgaattcatggtgtggcagcacacaaatgacaacgaagacatcaatccatatcattgtcggtacacctgtctgaacgaaaatttagacctggacaggcaacaggacagatatatactggggcgtgccttatatcacatggcgcagcgcagaggtttcctgagcaaccgactggatgcaacggaagaagatgaaaacggagcagtaaaaagtagcattgccaacctaaacaaggaaatagaagagaacggctgtaagtatttgggcgaatacttctacaaactatataaggagaaaggaaataccgtacgcatacgcacacgatataccgacagagagcaacattataagaaagagtttttcgccatttgcgaaaagcagaacctaccaaaggaaatggtagaagctctgacaagagccctatacttccaacgtcccttaaaaagtcagcggcagaacgtaggaaaatgtacatttgaaaaggggaagccaagatgcagcgattcacacccgacgtacgaaatataccgaatgctatctttcctgaacaatgttaagatacaagggccttgggatgaagaccttcgtccgctcaacgaagaagaattgaagcagatagacaagctattttacaggaagagcaagccaaacttcgactttgaagaaatagccaagaagattgcaggcaaaggcaaataccaatggaaaggagacaccaagacggatttgccatacaaattcaactatcgaatgtcgcaaggtgtgcccggatgccccacaattgcacaactgaaggaaatctttggcgagaattggaaagagaacattgccgaaacctatacacttaacacaaaaaaagatggcacacataagagtgcggacgaaatggttgatgatatctggaatgtactcttcttctttgacagcaaggacaaggtaaaggaatacggcattaaacatctacaattggacgaggatcaggccgataagttctcaaaaatacgcctcagccgtaattacgcctctctctcattgaaggcaattaggtatatccttccatttctacataaggggatgatatattccgatgcagttttctttgccaaaattcccgttatcattgggcaaagcctatggcagaaacgccagcacgaaataatggaggacctctcctacgccatacacatggcgatgaaagaaaagcaacgcattgatgatgctgtgaaagattacttacagaacaactttgatttgaaaccaggagcagcagatatgctctatcatccctctatgatagaaacctatcccgatgcacaaccagacaaaaacggagttgtattactgggcagccctcgcacaaacgccataaaaaacccaatggcaatgcgctctctgcatgaaatacgcaaggttatcaacactctgataaaggaaggtaaggttgcaccggatacagaaatacatatcgaatatgcaagggaactgaacaacgccaatatgcgagctgcgattcatgatttgcaacgcgaaaaagacaagaagcacaaagagtattcagaaaatatccgtaaactctacaaagaagcaaccggcaaggatatagaagctactgaaaccgacatactcaaattccagctttgggaagagcaaggtcacatttgcctatacacaggagatgagattggcattgcagatttcattggagcaaatcccagtttcgacatagagcatacaattccccgaagcgttgggggtgacagcacacaaatgaatatgaccctttgcaatacccattacaatagatttgtaaagaaagcctatatacccacgcaattagccgaccatgagtttattatgaccaggatagaaccctggaaggaaaaaatcgaagatctgacaaaacagattaaccagattaggacaagcaatatttgggacaaagcagccaaggatgcgaaaatcagaaagaagaaccgcctgttggaagaattaaagtactggaaaggcaaatacgacaggtttactatgaccgaagtgccagaaggctttgcccgtcggcagggagcaggaattggtcttatctccaaatatgcagcattgtacctaaagagctatttccatgatacacttcatcctgagcatagacaggtatatagcatcaagggaaccataacgtcagatttccgcaagatgtggggcatacaggaagaggacgaaaagaagtcaagagacagccacactcaccattgtattgatgccattgttattgcctgcataggcaagaacgaattcaacatgatgggacaatattatcatgtacaggagaaattcgaaaaaggcgagggagaaaaacccgagttcaagaagccttggccaacattcacccaggacctcaaaaaaattaaggaggaactggtggtagttcacgacacacctgacaacatgcctaaaaaagcaaccaagacgattgaaattagcaagggccggaaagtcgtcgcaaaaggagactgcgcacgcggcagccttcaccaagacacatattatggtgcaatagaaagagacggaaacataaagtatgttgtacgcaaaccgctgtcatcctttaccagcataaaggatttagacaacattgtggacgatgcagttcgccaggcaattctcaatgcgatagaaggcaaggacttcaagaaagcacttgcagaacccatctatatgaacaaagataaaggcattttgataaagaaagtaaggtgctatacaccttctataacaaaacctttacacatacgtacacatagggacatatcacaaaaggaatacaaacaacagttccatgttgctctggatagtaactattgtatggctatctatgagggagaagtgaaaggaaaaacaaaacgtacgtatgaaataataaacatgcttgatgcagctgcctatttcaaaaaaagctcgaatcttgccaaacaacatccaatagccccagagaacaaaaacgaacttaaatatagatgttctgtacatactggaacgcaagtcctgcttttgcaatcggatggcgaaaatataaatctcgagaacaaagacgatatagcaagacgtctttaccatgttgctgtgatgcgaagtgatggacgtatagttcttcgctacaatcaagatgcaagagacgctacaacattgaagaaagagcgtaaatctggtgcttacaaagtgggtgagcaatacagatccgaaataatgttatctctttctgactttcatgctctagtagaaggatatgacttcacaataacgcctttaggtgagataaaactaaaacattga

SEQ ID NO: 1556 - DNA, 4053 bp, Artificial Sequence
  OMNI-237 DNA sequence
atgggaaatcagtcaggacaggagtattatctggggcttgatatcggaacagactctgcggggtgggcagtaaccgatccggaataccgcattttaaggcggaaaggaaaatcactgtggggcgtgagactgttcgagtcggcaaatactgcggcagagcggagagcatttcgaacaaatcgtcggagacttcagagaaggcggcaacggatccttcttttgcaggaactgtttgcagaagaaatggaaaaagtggatgcaggtttttttcagagactgaatgacagcgcctgctgggcagaagagaaagaggaaccgcagatttattctctgtttggaggaaaagattacacagatgtggattattacagagaatatccgacgatctatcaccttcgcagtgcactgctccggggaaaacaggagtatgatctccggcttgtgtatctggcgctgcatcatctgatgaagcacagaggacatttcctgtttcagggaaatatcgaaagcgttacgtctttccagacaacttttcagaattttcaggactgtcttatggatgaattcggtctggagttggaatgcgattctgaggaaagattttcagagcttctgaaagacaggaaaatcggcaggatggaaaaatgccggcagctggaacttctgtgtcatgtacagaaaacggataagcagaagaaagaaatctttaaactgattacaggtatgaaggccggtttgtctgtgatatttgcagatgaggaactggcacagatcgactataataagattagtttcgcagaatcaggttatgaggaggcccgtctggcgctggaagaggagatccaggaacggacaggcattctggatgtctttcatgcggtttatagctgggcggttctggcagatattctggcagggggtgagtatgagggaaggccctgtctgtgcgtggcaaaagtaaatacatatgaaaagcaccgtcaggatctccgtctgctgaaaaaactggtgcgcgagtactgcccggaagattacaaatcttggttttcggttcctgtgaatgataactactgtgcttatatcggaacatttcgcaaaaacggaaaacgctgtgagacaaagcgctgcaatcaggaaacattttataaggcgttaaagaagctgttgggaaagatgtccgctgatcttccggaagcggctgacattttaaaagagacagaaaatgggacttttttgccattgcaggtcagtaaagataatggtgtgattccatatcaggttcataaaatggaattggagcggatactcgatcatgcggagaaatatctgccgtttcttctggagaaagacccggagtgtgggaaaacagtcagggaaaagatcgttgccctgtttgaattccggatcccctattatgtaggaccgctgaataccgccgcgggagaaaactgctggatggaaagaaaagaagaaggagcagtccgcccatggaattttgatcagaaggtggacagagacaaatctgcagagcggtttatccggagaatgacaaatcagtgtacctatctgattcatgagacggtattgccgaagcattccctgctttattcggaatttatggttctgaatgaactgaacaacgtaaagattcgtgatgaaaagcttccggtggaattaaaacagggcattgtacagaagctgtttaagacccggaaacaggtcacaggcaggaaactgctggattacctgacggcaaatggatacgatgtgaaaaaggatgaactgtccggatttgatgggaattttaaggcatctttgacttcttatcatactttaaaaaagatatttggtaaagaactggaaaaaagttccgtacagcagatggcagaggatatcatcttatggatcacattatatggagaagatcagaagatgctttgcagagtaatccggcagcattatggtgagatactgaaagaagaacagctgaaagagctgtccagattaaagtttcagggatggggcaggttatcccggagatttttgaatgaactggaaggcgttgactgtacgaccggagagtgtatgacgatcatgcagggacttaggaacacacagaataatctgatgcagcttttaagtcagcagtatacgtttgcccggatgatcgatgatgaaaatggaggttatcatgtggatgagattacctatgataatcttgtaaaagatattgtagcttctccatccataaaacgtgctgtgtggcagaccattcagattgtggaagaattgaaaaatgtaatgggatgcgagccgaaaaaagtatttgttgagatggcaagaggggaagaagagaaaaggcgtacggtttcccgtaaagacaaactgctggaagcatatgcggcaattaaagatgaagccagggactggcaggaggagttacagaaatattcagaaggagatctgaaagcaatcaagctgtatctgtattatacacaaatgggccagtgcatgtatacagggaagaaaatagaactgtcccagttaaacaacgcgaccatttgggacagggaccatatttatccgcagtcaaagacgaaagatgacagtctggataatctggtgctggtggacagaagagtcaatgcaaagaaaagcgacgggatgatatctccggagattcagcagaagatgcggggaacctggaaatatctgaaagagaaaggattgatctcggagaaaaagtatgaacgcctgacccggacaacgcctttgacggaagaagaactggcaggcttcatcaatcgtcagctggtggaaaccagacagtcttccaaagtggtggcaacgctgctgaaacgcatatatgaagaagcagagattgtgtatgtaaaggcgaaagcggtatcggatttcagaaaagagcatttgaaatatgtgaaagtacgtgatctgaatgactatcaccatgcgaaggatgcttaccagaatatcgtagtagggaatgtctatcatacaaagtttaccggtaatccgctgcgctggctgaaagaacatccggagcggaaatacagtctgaaccagatgttcaattatgatctggaagtgcacggaaagcctgtgtggaaacgggggaaggatggaagcattcgatgtgtgagtgagacactgaagcggaatgatattctgtttacaaggtatgcgttctgcaataagggagggctgtttaatcagacgattatgaaggcgccggaagataaggcaaaggccaaaggacttgttccgattaagaagggaaagaatccggaaaaatatggcggttttacatcggttaccccttctcattttatgctggtggcttcggaagggaaaaaaggaaaggagatccggacaattgagacggttccgctgtatcggaaaaaagaatttgagagagagccggagctgctgcttcagtattgccgggaattttacgggttaaaaaatccgagaattctgatttcctgtattaagaaaaatgcacgtctggttattaatggttttccaatgcacctgaaaggaagcacaggaaaacagctgatcttacagggggcggtacagctgtgcctggatgatgagaaagtggtgtacttgaaaaaagtgactaactatctggaagaaaatgccagaagaaaggataagcggacgctgctggaagtgaaggaaataacaggaattacaagtgaagcaaatataaatttatacgatttatttatcaaaaaactgagcagtacaatttatcaatatcgaccggctaatccaaaagataatcttctgaaagggcgtgagaagtttttaaatctgggactggctgagcaatgtgtggttttgggagagattttgcatctgtttcagtgtaaaccacttagtgcgaacttgatattgataggtgcggctggcaatacaggagttacaaagattgggaaggaaattagcaggtgcaattcagcaaagctggtttcccagtcagtgacaggtatttatgagcaggttgttgacctgctgaccatatga

SEQ ID NO: 1557 - DNA, 3237 bp, Artificial Sequence
  OMNI-291 DNA sequence
atgcaatggccttatggagtcggactggatattggtgttgcttcgattggatgggccactgttgccctggatgaaaaagcgcagccctgtggcttgattcgtctggggagccgtgtgttcaagcgcgcagagcagccgaagacaggagaaagtctggctgcaccaagacgggaggcccgcggcatgcgtcggcgtttgcgccgcagagctttgcgcaaacaggatttctatcagcttctggagaaaaatcatctgccggacaaggaagaactctcccggctgtttgcggcaggtggtttggaggatatttacgcgctgcgcaccagggccttggacgaggcggtgacaccgcaggaatttgcgcgcattctgctgcatctgatgcagcggcgtggattcaaaagcaaccgtctggcaggcaagtcgaaaaaagacggagtgcttctccaggcggtaacacaaaatcaactgcgcatgcaggaaaatggctataggactgttggcgagatgctgtataaagacccgctttttgcagcctgcaagcgcaacaagggggaaaattatctttccacagtcagtcgggaagaaattgccaatgaagcgcagctgctctttgaggcgcagcgcgaaaaaggccgggactgggcaacgccggaactggaagcggagtacctgtccattcttttgcgtcagcgctcctttgatcagggccccggcgggaacagcccatacaagggcggctgggcggaacgggttggcccttgtactttgctgccggaagaaaaacgtgcgttcaaaaacacatacagctttgagcgttttactttactgcaaaaaatcaataatatccggatcgtggaatccggtcagacccaggcactttcggatgaagaacgtcagaaagtaatggacctggcgtatcgtgtggagactgtgccgtataccctgattcgcaaagaactgcaatggccgcagacaatccggtttgccgatgtgcggtatgatgtggatgccgcaccggaagcagcagaaaaaaagtgcaaactcccgcccatgaaaggatatcatgcactgcgcaaggctctgggcacgcggcaggcagagcttcccttgtgggatgatgtgattaccattctggcatctcaccgggatgaaggcgtccggaaaaaactgctggaagaaaaaggtctgaccgcagaacaggtggaagctgttctcggcctggaattttccggtgtggggcatatttccctgaaggcttgtcagatgctggagccgtggctgctgcaggggctgaagtatgatcaggcatgcagcaaggccggattggattttcgcggtcatagcaatatcaaaaaaacgcctaagctgccggcttcagctccggagctgaaagacatcaccagcccggtggtgcgccgtgcggtgtcgcagaccatcaaactggtcaatgccatcatccgcgaaatggagcagccgcctgtgtggattcatattgaactggcccgggaactgagccgtacgtttgaggagcggcgcagggaccagaaatccatggaagacaacgccagacagaatgaatgcctgatgcaggagatccgggataccttccacaaactgaaccctacgggacaggatttgatcaagtaccgtctgtggaaagagcaggatgggatctgtgcttattctcagcaaagcattgcggtggaacggctgtttgaaaccggctatgctgaggtggatcatattgtgccgtacagcatcagttttgatgattcccgcagcaacaaagtgctggtgatggcggcggaaaaccatcagaaggggaatcggctgccgctgcaatatcttcagggagagcggcgcgaaaaatttattgtgtggacgaagtcgcaggtccgcaactatcgcaaacagaaaaatctgctgcgggaaacagtgagcgaagaagaacaaaacggtttcaagcagcgcaatctgcaagacacacagtatatggctcgctttttgctgaattatattcgggatcaccttgcatttgccgatcatccggctgcgggcaaacagcgggtcatggcgctggcaggtgcggttaccagccatcttcgcaaacgttgggggctgacaaaagtacgggcagacggtgaccttcatcacgcactggatgcggcagtcattgcctgtgccaccaagggcatggtacaacagatctccggctattatcaccgcattgaaggtgagtatcagcagtcctctgatgggacctattccgtgcatacccgtacaggcgaagtcttcccggccccgtggccgcatttccgggatgaactgatccagcggctgagccagaaaccacaggaaaatctgatgcagtggaatccggtgttttattccaattttgatgtggaatcgatccggcctgtttttgtgtcgcgtatgccgcagcataaggtgaccggggcggcccacaaggaaaccatcaaaagcccgaaagccctgcaggatggtctcctgatagtcaaacatcctctgacggatttgaaattgaataaaaagaccggcgagatcgaagggtactatcagccgtccagcgacaagctgttgtatgaggcgcttcgcgcccggttgctggaatttggcggggatggcaagaaagccttcacagaaccgttttacaagccaaaatcggacggcacgccggggccgctggtgcgcaaggtcaagctgtgcgaaaaggccagtctgtgtgttccggttcatggcggaaatggtgcggccgataacgacagcatggtgcgcgtggatgtgttctttgtcccgggggacgggtattacatggttcccatttacgtttccgacaccaaaaagaaagaactgcccaacagggctgtggtggcacataaggagttctctgcatggaaggtgatggaggaaaagaacttcctgttctcgctgtatgcggatgacttaattgaggtggctcgtaaaaaagtaatgccatttactatagtcaatagtgggagcactttacccccctcctatgaaacaactcatgagatggtgtattacggagggttagatatttcttctggtaatataaaagttacaatccatgataattcgtactctcttagaggactcggaattaagacgttaaatacccttcaaaaatatgaagtagatatcctaggcaatgtgagagaggtgaaaaaagaaaaacgtcaatcttttcgataa

SEQ ID NO: 1558 - DNA, 3294 bp, Artificial Sequence
  OMNI-308 DNA sequence
atggcttacgcaattggtttagacataggtattgcttctgtgggttatgcagtagttgcacttgattatgaagaaaatccatggggtatcatcaatatgggttctaggatttttgatgctgctgaaaatcctaaagatggggcttcgttagctgctcctagacgtgaggcacgtactggtagaagacgtttgcgtagaagaaaacatcgtattgaacgcattaaaaatttgtttttaaatactcaactgctgacaaaagaacaattagagaagttgtatgaaggtgttttatctgatgtttatgaattaagggttaaaggtttagatgaaaagctgacggctgaagaagttgcccgagttttattacatttggcacatcgtagaggctttaagtctaatcgtaaagcagaaaaaactgataaagaaaatggtgcattgcttactgccgttagtgaaaaccaaaaacgtatgaaagataagcaatatagaactattggtgagatgttccataaagacgagctttttgcagaatacaaacgtaataaaggtggtacatacttaacaacagttgctagagaaatgattgcggatgaagcaaaatatattattgctacacagagaagctttggtaataaaacattgacaatagagtttgaagaaagttatttagatatacttttaagtcaacgtaattttgatgaaggacctggtgggaatagtccgtatggcggtgaccaaattgaaagaatgataggaaactgcacatttgagaaaagtgagaagcgctgtgcaaaatcttgttattcttttgaatactttaatttgttgcaaaaaataaatcatttacgattgttagtagatggtgtgaaggttgaacttgatggttctcaaaaatctaaagttataaatcttgcaaaaacgaaggcagatttaaaatatagccaaatcaggaaggaattgggattaggttcgcaagttctattcaatgctctagtttataattctgatgtagaagaagtagagaaaaaatcaaaatttaattttatgcctgcgtatcatcaaatacgtaaagaattagatacagttgtcaagggaagaatagaagctatagacatagatactcttgatgaaataggaagaattttatcaacatataagagtgatgctaaacgtatagaagcatttagtaatttgaatataacttcaaaagatatggaagcattgcttaatattaatggtttaagaaaatttggtcacttatcattaaaagcgttaaagaagattacaccttttttggaacaaggaatgatttataacgaagcttgtactgctgcgggttatgagttccgtgggcatagtggttatgaaaaaactttcttactaccgtcacaaacagaagaaatggaaaatgttactagccctgtggcaagacgttctatttcacaatccattaaagttattaatgccattattcgagaacaaggagaaagtcctttatacataaacgtggaacttgcaagggaaatggcaaaagattttgatgaacgcaaaaaaataaaaaaggaaactgacgaaaaccaagcaagaaacgaaaggctgatagaaaaaattagaaatgaatttcataagcagaatcctacagggttggatttagtgaaattgaaattatgggaagaacaagatggtgttagtccttactcacaaaaacaaatagttattgaaaggttgtttgaaccagggtatgtagatgtagaccatattgttccctacagtatcagctttgatgatactatgaaaaataaagtattagtttttgcttctgaaaatagagaaaagggtaaaaacctgccagtagaataccttgcacgtaaaaaaggacaaaaggctgttgatgactttaaggtatgggttactagtaatgttagaaattacaaaaaacgacttaacttgttaaagtcagaagttacggaagaagacttaaataaatttaaagagcggaacttgcaggatacaaaaacaatttctcgttttatgtataactatatcagtgaccatcttatttttgcaccgtctgaaactggtaagaaaaagcgtgttactgctgtcaatggggcgataacttcctatttgcgtaaacgttggggtataaataaagtgcgtgctgatggagataagcatcacgctgttgatgctttggtaatagcttgcaccacagataaactgattaaagaccttagttatcactcaagatatctcgaaatggaatacgaagctggcgaaaaagaaagtttgtgggtaaatcgtaatactggcgaaatagttaaacgttttccttatccgtgggaagattttagagcagaactaatggcacgtttatcagatgctcctgaagttgctttaagaaaattgcaattgttatattaccaagataaggatttaagtaagattaaacctatttttgtgtctcgtatgccacggcgtaagattactggagcagcacataaagctacagttcgtggggcacgttacgttgataaggggattgtaatttccaagaaatctttgagtgagcttaaacttaataagggtggtgagattgaaaactattttgctcctgaaagtgatagactactgtatgaggctttgaaggaacaacttaaaaagtttaatggcaatgcaaaaaaagcttttgagcaagggttttataaacctaaagcagatggtacgcctggtccaaaagttaataaagtaaaggtttatgataagtctactttaaatgttgccgtgcaacaaggaactggtgccgctgataatgatagtatggtaagagttgacgtttataaggtagaaggagatggttattaccttgttcctatttacgtggcagatactttaaaaaaagagttacctaataaagcaatagtacaagggaaaacttatgagcaatggaaagaaatgtcaccagaagattttgttttctctttgtatcccaacgatttgataaagattactcacaaaaaggagataaactttagtagagttagcgaagatagtacattagctgattcttacaaaacaaaagaagagttcgtttattataaaaagactagtatttcaacagctagtataagcataataaacaatgataatacatatactattcctagtttgggtgtaaaaacattagcttctctagaaaaatatcaagttgatattttgggtaactataccaaggtaggaaaagaaaagctacaaacatttgctgcttatcaacaagctaaaaagaagccttga

SEQ ID NO: 1559 - DNA, 4044 bp, Artificial Sequence
  OMNI-103 Codon optimized for human cells
tctatcaagagcgactacttcctgggcctcgacatcggcacagattctatcggatgggccgtgacagaccccgagtaccacatcctgagaagaaagggcaaagccctgtggggcatcagactgttcgatgccgccaatactgccgccgagagaagaaccttccggaccagcagaagaagaatccagcggcggagacagcggatcagactgctgcaagagctgttcgccgaggaaatggttaagctggaccccggcttcttccagagactgagcgatagcgccttctggcaagaggacaagcaagagcagcagatctacagcctgtttacctgcgagaactacaccgacgtggactactacagagagtaccccaccatctaccacctgagaagcgccctgatccaagagaaaaaagagttcgacctgcggctgctgtatctggccctgcaccatctgatgaagcaccggggccactttctgttcaacggcagcatcaacaacgtgaccagcttccacaccaccttccagaccttcgccgactgcctgtacgacgagttcgacatcgagctggaatgcgacagcgaggacagattcgccgagatcctgaaggataagcacgccagaaagaccggcaagtgctctgagctggaaatcatctgccacatcgagaagtccaacaagcagctgaaagaactgttcaagctgatcaccggcatgaaggccagcctgagcgtggtgtttggagatgatgagctggccgagatcgagcacaacaagatcagcttcagcgagagcagctacgacgaagtgcggctggccctggaagatgagattcaagagagaaccggcatcctggacatcttccacgccgtgtattcttgggccatcctggccgatattctggaaggcggcgagtacgagggcaacagctatctgtctgtggccaaggtgtccacctacaagaagcacggcgacgacctgagactgctgagaacactcgtgcgcgagtactgccccgaccactacaagagctttttcagcgtgtccggcaaagagaactactgcgcctacgccggcacactgaagaagaacggcaagaagcagcccatcaagcggtgcagccaagaggacttctacaaggccctgaagaaactgctgaaccagatgcctaccgagcagcccgaagtgaaggatatcttcatcgagattgagaacggcaccttcctgcctctgcaagtgtccaaggacaacggcgtgatcccctaccaagtgaacaagatggaactcgagaagatcctgcagaacgccgaagagtacctgcctttcctgaagaacatcgacgaggaatgcggcaagaccgtgtccaagaagatcatcgacctgttcgagttcagaatcccctactacgtgggccctctgaataccgccaagggcgagaattgctggatggttcgaaaagaggccggcagaatctacccctggaacttcgatgagaaggtggacagagatcagagcgccgagaagttcatcagacggatgaccaaccagtgcacctacctgatccacgaggacgtggtgcctaagaacagcctgctgtactccgagttcatggtgctgaacgagctgaacaatgtgaagattcggagcgagaagctgcccgtggaactgaagcaggccatcgtgctggacctgtttaagaagcagaagcagatcacagggaagaagctgctcaactacctgaacgccaacggctacgacgtgaagaaagaggacctgagcggcttcgacggcaacttcaagtccagcctgtccagctacctgactctgaagaaggtgttcggagaggaactggacaagtacagcgtgcagcagatggccgaggacatcatcctgtggatcaccctgtatggcgacgatcagaaaatgctgcggagagtgatccggaagcactacgagcagcagctgtctgaggaacagatcctgagcctgagcaagctgaagttccaaggctggggcagactgtctagacggctgctctctgaaatggaaggcgtggactgtgaaaccggcgagtgcatgacagtgatgcagggcctgagaaacacccagaacaacctgatgcagctgctgagccagcagttcagcttcatggaactgatcgaggaagagaacgggaactactacgtcgacgagatcacctacgacaacctggtcaaggacatggtcatcagccctagcgtgaaaagggccgtgtggcagacagtgcagatcgtggaagaaatcaagggcgtgatgggatgccagcctaagaaaatcttcgtggaaatggcccgcagcgacgaagagaagaaacggaccgtgtctcggaaggatcggctgctggaagcctacgacgccatcaaggatgaggcccggcaatggcaagaagaactgcagaaatactccgacggcgatttcaaggccatcaagctgtacctgtactacacccagatgggccagtgcatgtacaccggcagaaaaatcgatctgtcccagctgaacgacgccaccgtgtgggatagagatcacatctaccctcagagcaagaccaaggacgacagcctggacaatctggtgctggtggatagatccgtgaatgccaagaaaagcgacggcatgctgagccccgagatccagcagagaatgagagccacctggaagtacctgaaagaaaagaagctcatcagcgagaagaagtacgagcggctgaccagagtgtcccctctgacagatgaagaactggccggcttcatcaaccggcagctggtggaaacaagacagagcagcaaagccgtggccacactgctgaagagggtgtacgatgaggccgagattgtgtatgtgaaggccgaggccgtgtctaacttccggcgggataacctggactacatcaaagtgcgggacctgaacgactaccaccacgccaaggacgcctaccagaacatcgtcgtgggcaacgtgttccacgagaagtttaccagcaatcccctgcggtggctgaaaaacaaccccaacaccaagtactccctcaaccagatgttcaacttcgacctggaaaagaacggcgtggtcatctggaagagaggcaaggccggctccattaagtgtgtggaagagacactgaagcggaacgacatcctgttcaccagatacgctttctgcaacaaaggcggcttctttaatcagatgctgaccgccgctccagaggataagacaaaggccaaaggcctggtgcctatcaagaaaggcatggaaacctggaaatacggcggctacaccagcgtgacccctagccactttatgctggtggccagcaaggacaagaagggaaaagagatccggaccatcgagacagtgcccctgtaccggtggaaagagttcaaagagaatcccgacgctctgctccagtactgcagagagttctacggcctgaaagagcccaaggttctgatcccttgcatcaagaagaatgcccggctggtcgtgaacggcttccctatgcacctgaagggcagcaccggaaaacagctgattctgcagggtgccgtgcagctgtgcctgaacaacgagaacatcaagtacctcaagaaagtgacgaagtacctcgagtacaacgcccagcggagagacaagagaaccctgctcgaagttcgggaagtgaccggaatcaacaaagaggaaaacatccagctgtacgatgtgttcgtggacaagctgagcaacacaatctaccagtacagacccgccaatcctaaggacaacctcatcaagggccgcgagaaattcatcgagcttggcctggctgagcagtgcgtggtgctgggagaagtgctgcatctgttccagtgcaagcccctgaccagcgatctgacactgatcggcggaagccctaacaccggcaccatcaagatcaccaagaccatcagcaactgcaacgtggtcaagctgctgtcccagtctatcgccggcgtgaaagtccgcgagatcaacctgctgatcatctga

SEQ ID NO: 1560 - DNA, 3288 bp, Artificial Sequence
  OMNI-124 Codon optimized for human cells
ggcaagatcatcggcctggacatcggaatcgcctctgttggatgggccgtgctggacaaggacagctacaagatcgtggaagccggcagcaatatctttcctgccgccgaggccagcaagaatcaagagagaagagagttcagaggcctgcggcggatgaccagaagaagaaggaccagactggacgacttcaagaagctgtggaagaactactacaagaacgagtacagcgtgacccctaacaacgtgaccgagctgaaagtgaagggcctgagcgagcagctgtccgagctggaactgtacaccgtgctgtactcctacctgaagcaccggggcatcagctacctggaagatgccgtggatgagggcaagaccggcagcgattatctgaagggactgcagaccaaccagaaagagatgaacaccaagtttcccagccagatccagtacgagcggctcgagaagtacggctgcttcagaggcgagaacaaggtgtccgatgacgaggtgctgagcaacgtgttcaccaccagcgcctacaagaaagaaatcgaggaactgatcagcgtgcaggacaacctgggcaacgagctggataaggacttcatcaagaagtactttgagatcttcagccggaagcggaagtactacgaaggccctggcaatgagctgagcagaaccgactacggcatctacaccaccaagatcgacgagaacggcaactacatcaccgacaagaacctgttcgagaagctgatcggcaagtgcagcgtgtaccccgaggaaatgagagccagcgccgcctcttacacagcccaagagttcaacgccctgaacgacctgaacaatctgatcgtgaacggccggaagctgaccaaagaggaaaagatcagcatcatcgagtgctacaagaccgagaaaaccatcaacgtgcggaagatcattaagaacgtgatcggggagaacatcgaccagctgagcggctaccggatggacaagaatgacaaagagctgtaccacaccttcgaggtgtacaacaagctgagaaaggccttcgaagaggccggcatctccttcgacttcagcagagaggactttgacgagatcggcagaatcctgacactgaacatcgagagagagggcatcgaggacgccatcagaatcagcaatctggtgctgtcctctgacgccgtggacgtgctgatctccgtgcggaaaaagaacgccagcctgttcagcaagtggcacagcttcagcctgaagatcatgaacgagctgatccccgagatgtacgagcagcctaagaaccagatggaactgctgagcgacatgaagtccatcaagagcaagatggatgagttcaagggcctcgaggatatccccgtggaaaccatcattgaggacatctacaaccccgtggtcaagcggagcatccggatcaccatcaagattatcaatgccctcaagaagaagtatagcgacatcgagaagatcgtgatcgagatgcccagagacaagaacagcgacgaggaaaaagagcggattaagaagttccagaaagaccaagagaatgagctgcccaacatcatcaagcgcgtgaaggacgagtacggcatcaccctgacacctgagctgatcgccagaaagaaaggactggcctttaagctgaaactgtggaacgagcagaacggcagatgcccctacagcggcagaatgatcgacattgagagcctgatcaacgacggcgatctgttcgaggtggaccacatcatccctctgagcatcagcttcgacgacagccggtctaacaagaccctggtgtacagagtggaaaatcaagaaaagaagaacaacacgcccttcatgtacctgagccggctgaacagacagtggaactacgacaagttcgccgccttcatcaaagaactgtacggcagcagcggcaaggaccggaagaaattccggaagaagatgaacaacttcctgttcaccgaggacattaacaagatcgatgtgctgaagggctttatcaaccggaacatcaacgacaccagatacgccagcagagtggtgctgaatagcctgcagggctacttccgggccaacgacaacaatgtgaaagtgtccgtgatcaacggcagcttcacccaccagatgagagtgaacctgagcctgggcaaagacagggatgagtcttttgcccaccacgctgtggacgccatgctgattgctctgagccagatgggctacgacgccttcagagccagacagaaagacatcgtggacttcgagacaggcgagatcatcaatggcagcgcctgggagaaccaggatgagatgtacaagacagtgctgtatcaagagaagtggaagaatatcaaggacaatatcatcgaagccgagccgaacatcaagtactggcacaaggtggacaaaaagtgcaaccggggcctgtgcaaccagaccatctacagcacacggaagtgcgacgataagatctggaaagtggacaagctgaacatccgcgacaaggacgacgtgaagaaactgatctacatgatccagaacggaaaagaggacaaaatcctgatggcccagaacgaccccaagaccttcgccaacctgaagtacatcctggatgagtactccgactgcgagaaccccttcgtgtcttacgagaaagaaaccggcgacctcgtccggaagtacagcaagaagcacaacggccccaagatcacctacctcaagtacatggtggaagaagtcaacagctgcatcgacatcagccacaaatacggccacgagaagggcagcaagaaagtgtttctggcccagctggtgcccttccggaccgacgtgtactacaacaacaactccggcaagtactacctgatcggaatcaagcagtccgacatcaagtgcaagggcaaccagtacatcatcgacgaagagaagtacaccgacaacctgattcagaacggcctgctgaaagagggccagaagatcgaggacctggaaaagctgggctacctgttcaagttcagcatcttcaagaacgataccatcgagtttgagaagaagggcgagctgattaagtgccggttcctgtctcagaactccgcctccaagaaccggatcgaagtgaagcccatcggcaagaaggactacgagagccggaatctgcctacactgagcaccaccacagtgatcaacaaatacgtcaaggacatcctcgggaacgaaaaacaggtgtccggcgagaagttcgagatcattgtggacagcaag

SEQ ID NO: 1561 - DNA, 3324 bp, Artificial Sequence
  OMNI-127 Codon optimized for human cells
ggaaagctggtgctgggcctcgatatcggcatcacatctgtcggctacggcgtgatcgacatcgacaacaacaagttcgtggattacggcgtgcggctgttcaaagagggcgacagcaacgacaacgagaagcggagaagctacagaagcggcagacggctgcggcggagaaagaagaacagactggacgacatgaagtccctgctgctgtctgccggcatcctgaccaaggactacaagcccctgaacaacatctacgagatcagagtgaagggcctgaccgagaagctgaccaacaatgaactggccgctgctctgctgcacatcaccaagaatagaggcaccaccgtggaagccctggaaaccgccgatgctgacaaagaagccgccggaacaaaggacgtgctggccaagaatgccaaggacctggaacagttcgagtacgtgtgcaaggtgcagctgtaccggctcgagaagtacggcaatatccggggcaccgagaacaacttcaacaccagcgactacgtgaaagaggccagagccatcctgaagtgccaggacatcgacgtggaactgagcgagagcatcgtgaacatcatcagccggcggagagagtactacgaaggccctggaagcgagaagtcccctaccaaatacggcagatggatcgagaagggcgccgagcctatcgacctgattgagaagatgcggggcaagtgcagcatctaccacgatgagctgagagcccctaagagcagctacagcgccgagctgttcaacctgctgaacgacctgaacaatctgagcatcaacggcgagaaactgacccagaacgagaaagagatcgtcatcgaggaagtgaacaacaacggcaagatcaccatcgacaagctgaagaagctgctgaacatcaacgacagcaaggacgtggtggaaggcttccggatcaacaagagcaacgagctgctgctgaccaatatggctatgtacgaccagttcaagaagatctttgacaagcacaaagaggacgcctaccagcgggacaagtacatcatcgaccagatcgccgagatcaacaccgccaagaagggcatcaaagagagatgcgacgccatcgagaagcagttccccaacttcgactacaacctgatccgcgacctggccgagatgaagaagatgacccagtaccacagcctgagcctgaaggccatccgggaaatcaacaaagagatgatcaagagcccgtacaaccagatgcaggccatccatgaactgggcctgcagcactacagaaagtcctacaagggcatgaagaaaatccaggtggacgaggaagccattctgagccctgtggtcaagagagcccagcgggaaaccttcaaagtgatcaacagactgagagagatctacggcgagttcgagagcattgtggtggaaaccaccagggacaagaactccaaagagaagaaagagcggattaacaagacccagaagtattacgaggaccggaacaagaaagtggacgaactgctgaaagagagaggctacaaccccgacaccattaacaacaagaccaagatgaaggtcaaactgtacctggaccagaacggcaagtccgcctacaccatgacacccctgaagctgaacctgatcatcaccaatcctcacgcctatgagatcgaccacatcatccctctgtccatcagccaggacgacagcttcaacaacaaagtgctgatcacccacgaagagaatcaagtcaagggcaacaagacacccatctacacctacctcaacaaccagtttaagaactgcgatatcaacaagtacaaagaaagcgtgaagaacaaccacaacttcagccggaagaaaaagttctacatgctgtacgagaaggacatcacgaagtacagcaacatgaaggagtttatcgcccggaacctggtggacaccagctacgcttctagagtggtgctgaacaccctgaccagctacttccgggacaacgacatcagcaccaacgtgcacacaatcagaggcagcgccaccagcctgtacagaaagagaatcggcctgaagaaggaccgcgacaaggatttcctgcaccacgccattgatgcactgatcgtggccagcatcaagaagcagagcatctttcggaagtacctgatcaagtacaacctcagcgagctgtacaacgaaaagaccggcgagctgatcaaggtggaagaggactctaaagtgatcgaccccgtgttcatcgagttcatcagcaacctcaagacaatctacgagcagagctaccagtactacaacggctacatctctcggaacaaaatgaagttcaagcccatcaagatcagccacaaggtggacacaaagcccaaccggaagatcagcgacgaaaccatctacagcacccggaacgtgaacggcgtggaacgggtcgtgaagaaagtgaaggatatctacgaccccaagttcgacaaactgaccaacgatatcatcaacaatcagtatcacaacaagtggatcatggccgagaaggatgagcagaccttcgagatcatcaagtccatcgtgatggcccacttcaatacctacaaagaagataaggacatctacaagtccaagagcaagaaaggcgtgaccacatggtccctgaagggcagcaacaaccctctgagccagtttaaagaggaacacgggaagatccgcaagtactctaagaaaggcaacggccccgagatcaccagcatgaagtactatgaggacgagctgggcaaccacgtgaacattaccaaaaagtacaacgtggacaacaagaacgtggtcctgctgcagatcagcccctacagaaccgacttctacgtgtcccctaagggcaagtacaagtttgtgaccatccggtactgcaacgtgttctacaaagagagcaacaagaagtatctgatcgataagaagtggtacgaagaggaaaagaaaaagaagaagatcgacgactcctaccagttcgtgtgctccctgcaccatgatgagatcctgggcgtgaagctggaagatggcaagccctacatctatgacgagtccaacgagaacgacgagggcctgaaaagataccacaagggccagtacgagattctgaagttcaccgccaccaacgacgacaagaagaatatcatcgagatcaagcccacgtacacctactgccagaagcagctgaagagaagctgcggcaccttcatcaagctgactaagttcgccaccgatgtgctgggcaatatgtacgaagtgaaagacaacgtgctgaagctcgagttcgac

SEQ ID NO: 1562 - DNA, 3654 bp, Artificial Sequence
  OMNI-218 Codon optimized for human cells
ggtaccagcaagaagagaatcctgggcctcgacaccggcacaaattctcttggatgggccgtcgtggaccaagaggaaaatggccagtacaccctgatcgacaagggcgtcgtgatcttccaagagggcgtgaagatcgagaaaggcgtggaatctagcaaggccgccgagagaacagaacacagagccctgcggagacactacttccggcggagactgagaaagatcgaggtgctgagagtgctggtcaagtacaatctgtgccccaccatcaccgaagaggacctgaatctgtggaagctgcacaagacataccccagaaaggacgagttcatggtctggcagcacaccaacgacaacgaggacatcaacccctaccactgccggtacacctgtctgaacgagaacctggacctggacagacagcaggaccggtatattctggggagagccctgtatcacatggcccagagaagaggcttcctgagcaacagactggacgccaccgaagaagatgagaacggggccgtgaagtcctctatcgccaacctgaacaaagagatcgaggaaaacggctgcaagtacctgggcgagtacttctacaagctgtacaaagaaaagggcaacaccgtgcggatcagaaccagatacaccgacagagagcagcactacaagaaagagttcttcgccatctgcgagaagcagaacctgcctaaagaaatggtggaagccctgacacgggccctgtacttccagaggccactgaagtcccagagacagaacgtgggcaagtgcaccttcgagaagggcaagcccagatgcagcgactctcaccccacctacgagatctaccggatgctgtccttcctgaacaacgtgaagattcaaggcccctgggacgaagatctgcggcccctgaatgaggaagaactgaagcagatcgataagctgttctaccgcaagagcaagcccaacttcgacttcgaggaaatcgccaagaagatcgccggcaagggcaagtaccagtggaagggcgataccaagaccgacctgccttacaagttcaactacagaatgagccagggcgtgcccggctgtcctacaattgcccagctgaaagagatctttggcgagaactggaaagagaatatcgccgagacatacaccctcaacaccaagaaggacggcacccacaagagcgccgacgagatggtggacgacatctggaacgtgctgttcttcttcgacagcaaggacaaagtgaaagagtacggcatcaagcaccttcagctggacgaggaccaggccgacaagttcagcaagatccggctgagcagaaactacgccagcctgtctctgaaggccatccggtacatcctgccttttctgcacaagggcatgatctacagcgacgccgtgttcttcgctaagatccccgtgatcatcggccagagcctgtggcagaaacggcagcacgagatcatggaagatctgtcctacgccatccatatggccatgaaggaaaagcagcggatcgacgatgccgtgaaggactacctgcagaacaacttcgatctgaagcctggcgccgctgacatgctgtatcaccccagcatgatcgagacttaccccgacgctcagcccgacaaaaatggcgttgtgctgctgggcagccccaggaccaatgccattaagaaccccatggctatgcggagcctgcacgagattcggaaagtgatcaacaccctcatcaaagagggcaaagtcgcccctgacaccgagatccacatcgagtatgcccgcgagctgaacaatgccaacatgagagccgccatccacgacctgcagcgcgagaaggataagaagcacaaagagtacagcgagaacatccggaaactctacaaagaggccaccggcaaggacatcgaggccacagaaaccgacatcctgaagttccagctgtgggaagaacagggacacatctgcctgtacaccggcgacgagatcggaatcgccgactttatcggcgccaatcctagcttcgacatcgagcacacaatccccagaagcgtcggcggcgatagcacccagatgaacatgaccctgtgcaacacccactacaaccgcttcgtgaagaaggcctacattcccacacagctggccgaccacgagtttatcatgacccggatcgagccctggaaagaaaagattgaggatctgaccaagcagatcaaccagatccggaccagcaacatctgggacaaagccgccaaggacgccaagatcagaaagaagaaccggctgctcgaggaactgaagtactggaaggggaagtacgaccggttcaccatgacagaggtgcccgagggatttgccagaaggcaaggcgccggaatcggcctgatttctaagtacgccgctctgtacctgaagtcctacttccacgacacactgcaccccgagcaccgccaggtgtactctatcaagggcaccatcacaagcgatttccggaagatgtggggcatccaagaagaggacgagaagaagtccagggacagccacacacaccactgcatcgacgccatcgtgatcgcctgcatcggcaagaacgagttcaacatgatgggccagtactaccacgtgcaagagaagttcgaaaaaggcgagggcgagaagcccgagttcaagaagccttggcctaccttcacacaggatctgaagaagatcaaagaggaactcgtcgtcgtccacgacacccctgacaacatgcctaagaaggccaccaagaccatcgagatcagcaagggcagaaaggtggtggccaagggcgattgtgccagaggatctctgcaccaggatacctactacggcgccatcgagcgcgacggcaacatcaaatatgtcgtgcggaagcccctgagcagcttcaccagcatcaaggacctggataacatcgtggatgatgccgtgcggcaggccatcctgaatgccatcgagggaaaagacttcaagaaggccctggctgagcccatctacatgaacaaggataagggcatcctgatcaagaaagtgcggtgctacacccctagcatcaccaagcctctgcacatcagaacccaccgggacatcagccagaaagagtataagcagcagttccacgtggccctggacagcaactactgcatggccatctacgagggcgaagtgaaaggcaagaccaagcggacctatgagatcatcaacatgctggacgccgctgcctacttcaaaaagagcagcaacctggccaagcagcaccctatcgctcccgagaacaagaatgagctgaagtaccggtgcagcgtgcacacaggcacacaagttctgctgctgcagagcgacggcgagaatatcaacctggaaaacaaggacgatatcgccagacggctgtaccatgtggccgtgatgagatccgatggcagaatcgtgctgcggtacaaccaggacgctagagatgccaccacactgaagaaagagcgcaagagcggcgcctacaaagtgggcgagcagtacagatccgagattatgctgagcctgagcgacttccacgctctggtggaaggctacgacttcacaatcacccctctgggcgagatcaagctgaagcacggatcc

SEQ ID NO: 1563 - DNA, 4059 bp, Artificial Sequence
  OMNI-237 Codon optimized for human cells
ggtaccggcaatcagagcggccaagagtactacctgggcctcgatatcggcacagattctgccggatgggccgtgaccgatcctgagtacagaatcctgcggagaaagggcaagagcctgtggggcgtcagactgtttgagtctgccaacacagccgccgagcggagagccttcagaacaaacagaagaaggctgcagcggcggagacagcggattctgctgctgcaagagctgttcgccgaagagatggaaaaggtggacgccggcttcttccagagactgaatgatagcgcctgctgggccgaagaaaaagaggaaccccagatctacagcctgttcggcggcaaggactacaccgacgtggactactacagagagtaccccaccatctaccacctgagatctgccctgctgcggggcaagcaagagtacgatctgagactggtgtatctggccctgcaccacctgatgaagcaccggggacattttctgttccaaggcaacatcgagagcgtgaccagcttccagaccacctttcagaacttccaggactgcctgatggacgagttcggcctggaactggaatgcgacagcgaggaaagattctccgagctgctgaaggaccggaagatcggcagaatggaaaagtgccggcagctggaactgctgtgccacgtgcagaaaaccgacaagcagaagaaagagatcttcaagctgatcaccggcatgaaggccggactgagcgtgatctttgccgatgaggaactggcccagatcgactacaacaagatcagcttcgccgagagcggctacgaagaagccagactggccctggaagaggaaatccaagagagaaccggcatcctggacgtgttccacgccgtgtattcttgggccgtgctggctgatattctggctggcggagagtacgagggcagaccttgtctgtgcgtggccaaagtgaacacctacgagaagcacaggcaggacctgcggctgctgaaaaagctcgtgcgggaatactgccccgaggactacaagagctggttcagcgtgcccgtgaacgacaactactgtgcctacatcggcaccttcagaaagaacggcaagagatgcgagacaaagcggtgcaatcaagagacattctacaaggccctgaagaaactgctgggcaagatgagcgccgatctgcctgaagccgccgacatcctgaaagagacagagaacggcacctttctgcccctgcaagtgtccaaggacaacggcgtgatcccctaccaggtgcacaagatggaactcgagagaatcctggaccacgccgagaagtacctgcctttcctgctggaaaaggaccccgagtgtggcaagacagtgcgggaaaagatcgtggccctgttcgagttcagaatcccctactacgtgggccctctgaataccgccgctggcgagaattgctggatggaaagaaaagaagagggcgccgtcagaccctggaacttcgatcagaaggtggacagagacaagagcgccgagagattcatcagacggatgaccaaccagtgtacctacctgatccacgagacagtgctgcccaagcacagcctgctgtactccgagttcatggtgctgaacgagctgaacaacgtgaagatccgggacgagaagctgcccgtggaactgaagcagggaatcgtgcagaagctgttcaagacccggaagcaagtgaccggcagaaagctgctggactacctgaccgccaacggctacgacgtgaagaaggatgagctgagcggcttcgacggcaactttaaggccagcctgaccagctaccacacgctgaagaagatctttggcaaagagcttgagaagtccagcgtgcagcagatggccgaggacatcatcctgtggatcaccctgtacggcgaggaccagaaaatgctgtgcagagtgatccggcagcactacggcgagattctgaaagaagaacagctgaaagaactgagccggctgaagtttcaaggctggggcagactgagcagacggttcctgaatgaactggaaggcgtggactgtaccaccggcgagtgcatgacaatcatgcagggcctgagaaacacccagaacaacctgatgcagctgctgagccagcagtacaccttcgccagaatgatcgacgacgagaacggcggctatcacgtggacgagatcacctacgacaacctggtcaaggacatcgtggcaagccctagcattaagagggccgtgtggcagaccatccagatcgtggaagaactgaagaacgtgatgggctgcgagcccaagaaagtgttcgtggaaatggccagaggcgaggaagagaagcgcagaaccgtgtccagaaaggacaagctgcttgaggcctacgccgccatcaaggatgaggccagagattggcaagaggaactgcagaagtacagcgagggcgacctgaaggccatcaagctgtacctgtactacacccagatgggccagtgcatgtacaccggcaagaagatcgagctgtcccagctcaacaacgccaccatctgggacagagatcacatctaccctcagagcaagaccaaggacgacagcctggacaatctggtgctggtggatcggagagtgaacgccaagaaaagcgacggcatgatcagccccgagatccagcagaagatgagaggcacctggaagtacctcaaagagaagggcctgatcagcgagaagaagtacgagcggctgaccagaaccacacctctgaccgaagaagaactggctggcttcatcaacagacagctggtggaaacccggcagagcagcaaagtggtggccactctgctgaagcggatctacgaagaggccgagatcgtgtacgtgaaggccaaggccgtgtccgacttccggaaagagcacctgaaatacgtcaaagtgcgggacctgaacgactaccaccacgccaaggatgcctaccagaacatcgtcgtgggcaacgtgtaccacaccaagttcaccggcaatcctctgcggtggctgaaagagcatcccgagagaaagtacagcctgaaccagatgttcaactacgatctcgaggtgcacggcaagcccgtgtggaaaagaggcaaggacggcagcatcagatgcgtgtccgagacactgaagaggaacgacatcctctttacccgctacgccttctgcaacaaaggcggcctgttcaaccagaccatcatgaaggctcccgaggacaaggccaaagccaaaggcctggtgcctatcaagaagggaaagaaccccgagaaatacggcggctttaccagcgtgacccctagccactttatgctggtggcctctgagggcaagaaaggcaaagagatccggaccatcgagactgtgcccctgtacagaaagaaagagttcgagagagagcctgagctgctgctccagtactgcagagagttctacggcctgaagaaccctagaatcctgatctcctgcatcaagaagaacgcccggctggtcatcaacggcttccctatgcatctgaagggctctaccggcaagcagctgattctgcagggtgcagtgcagctgtgcctggatgatgagaaggtggtgtacctgaaaaaagtgaccaactacctcgaggaaaacgccagacggaaggacaagagaacactgctggaagtgaaagagattaccggcatcaccagcgaggccaacatcaacctgtacgacctgttcattaagaagctgagcagcacgatctaccagtacagacccgccaatcctaaggataacctgctgaaaggccgcgagaagttcctgaacctgggacttgctgagcagtgtgtggtgctgggagagatcctgcacctgttccagtgtaaacccctgagcgccaacctgatcctgatcggagccgctggaaataccggcgtgaccaagatcggaaaagaaatcagccggtgcaacagcgccaagctggtgtctcagtctgtgacaggcatctacgaacaggtggtggatctgctgaccatcggatcc

SEQ ID NO: 1564 - DNA, 3243 bp, Artificial Sequence
  OMNI-291 Codon optimized for human cells
ggtacccagtggccttatggcgttggcctggatatcggagtggcctctattggatgggccaccgtggctctggatgagaaagctcagccttgcggcctgatcagactgggcagcagagtgtttaagagagccgagcagcctaagaccggcgaatctctggctgcccctagaagagaagccagaggcatgcggagaaggctgagaagaagggccctgcggaagcaggatttctaccagctgctggaaaagaaccatctgcctgacaaagaggaactgagcagactgtttgccgctggcggcctggaagatatctacgccctgagaactagagccctggacgaggctgtgacccctcaagagtttgccagaatcctgctgcacctgatgcagcggagaggcttcaagtctaacagactggccggcaagagcaagaaagacggcgttctgctgcaggccgtgacacagaaccagctgagaatgcaagagaacggctaccggacagtgggcgagatgctgtacaaagatcctctgttcgccgcctgcaagcggaacaagggcgagaattacctgagcaccgtgtccagagaggaaatcgccaacgaggctcagctgctgtttgaggcccagagagagaagggcagagattgggccacacctgagctggaagccgagtacctgtctatcctgctgagacagcggagcttcgatcaaggccctggcggcaattctccttacaaaggcggatgggccgagagagtgggaccttgtacactgctgcctgaggaaaagcgggccttcaagaacacctacagcttcgagagattcaccctgctgcagaagatcaacaacatccggatcgtggaaagcggccagacacaggccctgagcgacgaagaaagacagaaagtgatggacctggcctacagagtggaaaccgtgccttacacactgatccggaaagaactgcagtggccccagaccatcagatttgccgacgtccgctacgatgtggatgccgctcctgaagccgccgagaagaagtgcaaactgcctcctatgaagggctaccacgctctgagaaaagccctgggaacaagacaggccgagctgcctctgtgggacgatgtgattacaatcctggccagccacagagatgaaggcgtgcggaagaaactgctggaagaaaagggcctgaccgccgaacaggtggaagctgtgctgggactcgaattttctggcgtgggccacatcagcctgaaggcctgtcaaatgctggaaccatggctgctccagggcctgaagtacgatcaggcctgttctaaggccggactggacttcagaggccacagcaacatcaagaaaacccctaagctgcccgccagcgctcccgagctgaaggatattacatctcctgtcgtgcggagagccgtgtctcagacaatcaagctggtcaacgccatcatcagagagatggaacagcctcctgtgtggattcacatcgagctggccagagagctgagccggaccttcgaagaacggcggagagatcagaaatccatggaagataacgcccggcagaacgagtgcctgatgcaagaaatccgggacaccttccacaagctgaaccctaccggccaggacctgatcaagtaccggctgtggaaagagcaggacggcatctgcgcctactctcagcagtctatcgccgtggaaagactgttcgagacaggctatgccgaggtggaccacatcgtgccctacagcatcagcttcgacgacagcagatccaacaaggtgctggtcatggccgccgaaaaccaccagaagggcaatagactgcccctgcagtacctccagggcgagagaagagaaaagttcatcgtgtggaccaagagccaagtgcggaactaccggaagcagaagaacctgctgcgcgagacagtgtccgaggaagaacagaacggcttcaagcagcggaacctgcaggacacccagtacatggcccggtttctgctgaactacatcagggatcacctggccttcgccgatcatcctgccgctggaaaacagcgagttatggctctggctggcgccgtgacaagccacctgagaaaaagatggggactgaccaaagtccgcgccgacggcgatctgcatcatgcactggatgctgccgtgatcgcctgtgccacaaaaggcatggtgcagcaaatcagcggctactaccaccggatcgagggcgaataccagcagtcctctgatggcacatacagcgtgcacaccagaaccggcgaagtgtttcctgctccttggcctcacttccgggacgagctgattcagagactgtcccagaagcctcaagagaacctcatgcagtggaaccccgtgttctacagcaacttcgacgtggaatccatcagacccgtgttcgtgtccaggatgccccagcacaaagtgacaggcgccgctcacaaagaaaccatcaagagccccaaggctctgcaggatggcctgctgattgtgaagcaccctctgaccgacctgaagctgaacaagaaaacaggcgagatcgaggggtactaccagcctagcagcgacaagctgctgtacgaagctctgagagcccgcctgctggaatttggcggagatggcaagaaggcctttaccgagcctttctacaagcccaagtccgacggaacacctggacctctcgtgcggaaagtgaaactgtgcgagaaggccagcctgtgtgtgcctgttcatggtggaaacggcgctgccgacaacgattctatggtccgagtggacgtgttcttcgtgcccggcgacggctactatatggtgcccatctacgtgtccgacaccaagaagaaagagctgcccaatagagccgtggtggcccacaaagagttcagcgcctggaaagttatggaagagaagaatttcctgttcagcctgtacgccgacgacctgattgaggtggcccggaaaaaagtgatgcccttcaccatcgtgaacagcggcagcacactgcctccaagctacgagacaacccacgagatggtgtactacggcggactggatatcagcagcggcaatatcaaagtgaccatccacgacaacagctactccctgagaggcctgggcatcaagaccctgaacaccctgcagaaatatgaggtggacatcctgggcaacgtgcgggaagtcaagaaagagaagcggcagtccttccggggatcc

SEQ ID NO: 1565 - DNA, 3300 bp, Artificial Sequence
  OMNI-308 Codon optimized for human cells
ggtaccgcctatgccatcggcctggatatcggaatcgcctctgtgggatatgccgtggtggccctggactacgaggaaaacccttggggcatcatcaacatgggcagcagaatcttcgacgccgccgagaatcctaaggatggcgcttctctggccgctcctagaagagaagctagaaccggcagacggcggctgcggagaagaaagcacagaatcgagcggatcaagaacctgttcctgaacacccagctgctgaccaaagagcagctggaaaagctgtacgagggcgtgctgtccgacgtgtacgagctgagagtgaaaggcctggatgagaagctgacagccgaagaggtggccagagttctgctgcatctggcccacagaagaggcttcaagagcaaccggaaggccgagaaaaccgacaaagagaatggcgccctgctgaccgccgtgtccgagaaccagaaacggatgaaggacaagcagtaccggaccatcggcgagatgttccacaaggatgagctgttcgccgagtacaagcggaacaaaggcggcacctatctgaccaccgtggccagggaaatgatcgccgacgaggccaagtacatcattgccacacagcggagcttcggcaacaagaccctgaccatcgagttcgaggaaagctacctggacatcctgctgagccagcggaactttgatgaaggccctggcggcaatagcccttacggcggagatcagatcgagagaatgatcggcaactgcaccttcgagaagtccgagaagagatgcgccaagagctgctacagcttcgagtacttcaacctgctgcagaagatcaaccacctgagactgctggtggacggcgtgaaggtggaactggatggcagccagaaaagcaaagtgatcaacctggccaagaccaaggccgacctgaagtacagccagatccggaaagaactcggcctgggcagccaggtgctgttcaatgccctggtgtacaacagcgacgtggaagaagtggaaaagaagtccaagttcaacttcatgcccgcctaccaccagattagaaaagaactggacaccgtggtcaaaggccggatcgaggccatcgacatcgatacactggacgagatcggcagaatcctgagcacctacaagagcgacgccaagagaatcgaggccttcagcaacctgaacatcaccagcaaggacatggaagccctgctcaacatcaacggcctgagaaagtttggccacctgagcctgaaagccctgaagaagatcaccccatttctggaacagggcatgatctacaacgaggcctgtacagccgccggatacgagtttagaggccacagcggctacgaaaagaccttcctgctgcctagccagaccgaagagatggaaaacgtgacatctcctgtggccaggcggagcatcagccagagcatcaaagtgattaacgccatcatcagagagcagggcgagagccctctgtacatcaatgtcgaactggcccgcgagatggccaaggacttcgacgagcggaagaagattaagaaagagacagacgagaatcaggcccggaacgagcggctgatcgagaagatcagaaacgagtttcacaagcagaaccccaccggcctggacctggttaagctgaagctgtgggaagaacaggatggcgtgtccccttactctcagaaacagatcgtgatcgagaggctgttcgagcccggctatgtggacgtggaccacatcgtgccttacagcatcagcttcgacgacaccatgaagaacaaggtgctggtgttcgccagcgagaaccgcgagaagggcaagaatctgcccgtggaatacctggccagaaaaaagggccagaaagccgtggacgacttcaaagtgtgggtcacctccaacgtgcggaactacaagaagcggctgaacctgctgaagtccgaagtgaccgaggaagatctgaacaagttcaaagagcggaacctgcaggacaccaagaccatcagccggttcatgtacaactacatcagcgaccacctgatcttcgcccctagcgagacaggcaagaagaaaagggtcaccgccgtgaacggcgccatcacctcctatctgagaaagcgctggggaatcaacaaagtccgcgccgatggcgataagcaccatgccgtggatgccctcgtgatcgcctgtaccaccgacaagctgatcaaggacctgagctaccacagccggtatctggaaatggaatacgaggccggcgagaaagaaagcctgtgggtcaacagaaacaccggcgagatcgtgaagcggttcccctatccttgggaagatttcagagccgagctgatggccagactgtctgatgcacctgaagtggccctgcggaaactgcagctgctgtactaccaggacaaggacctcagcaagatcaagcccatcttcgtgtccagaatgcctcggagaaagatcaccggcgctgcccacaaagctacagtgcggggagccagatatgtggacaagggcatcgtgatcagcaagaagtccctgagcgagctgaagctcaacaaaggtggcgagatcgagaactacttcgcccctgagagcgacagactgctgtatgaggccctgaaagaacagctgaagaagtttaacggcaacgccaagaaggctttcgagcagggattctacaagcctaaggccgacggcacccctggacctaaagtgaacaaagtgaaggtgtacgacaagagcaccctgaacgtggccgttcaacaaggcacaggcgccgctgacaacgattctatggttcgagtggacgtgtacaaggtggaaggcgacggctactacctggtgcctatctatgtggccgacacactgaagaaagagctgcccaacaaggccattgtgcagggcaagacctacgagcagtggaaagagatgagccccgaggacttcgtgttctctctgtaccccaacgacctgatcaagatcacgcacaagaaagaaatcaacttcagccgggtgtccgaggacagcacactggccgatagctacaagaccaaagaagagttcgtctactacaaaaagaccagcatctccaccgcctccatcagcatcattaacaacgacaacacctacacaatccccagcctgggcgtgaaaacactggccagcctggaaaagtaccaggtggacattctgggcaactacaccaaagtgggcaaagaaaagctgcagaccttcgccgcctatcagcaggccaagaaaaagcccggatcc

SEQ ID NO: 1566 - RNA, 107 nt, Artificial Sequence
  OMNI-103 sgRNA scaffold
guuugagaguaguguaagaaauuacacuacaaguucaaauaaaaauuuauucaaauccauuugcuacauuguguagaauuuaaagaucuggcaacagaucuuuuuuu

SEQ ID NO: 1567 - RNA, 119 nt, Artificial Sequence
  OMNI-124 sgRNA scaffold
guuuaaguaccuggagaaagaaauuucuucagaccuacuuaaauaaggauuuauuccgaaagaauuauuguaaaugaagaaaauaaggcgagaaaggaugcugacgggcauccuuuuuu

SEQ ID NO: 1568 - RNA, 87 nt, Artificial Sequence
  OMNI-127 sgRNA scaffold
guuuaguuaccauauggaugaaaaucuauaugaccuaacuaaacaaggguuuaucccggauucggcuccuuuauaggagccuuuuuu

SEQ ID NO: 1569 - RNA, 92 nt, Artificial Sequence
  OMNI-218 sgRNA scaffold
guugugguuugaugaugaaaaucaucaaaucacaauaaggcuauaugccgaaggaugaauuccuaucgccucucuucuggagaggcuuuuuu

SEQ ID NO: 1570 - RNA, 107 nt, Artificial Sequence
  OMNI-237 sgRNA scaffold
guuugagaguaguguaagaaauuacacuacaaguucaaauaaaaauuuauucaaauccuucugcugcauugugcagaaucuaaagaucuggaaacagaucuuuuuuu

SEQ ID NO: 1571 - RNA, 98 nt, Artificial Sequence
  OMNI-291 sgRNA scaffold
guuguagcucccugacagugaaaacugucaugguugcuacaauaagguaguauaccguaaagcucugacgcccugcuugcagggcgucaucuuuuuuu

SEQ ID NO: 1572 - RNA, 99 nt, Artificial Sequence
  OMNI-308 sgRNA scaffold
guuguaguucccuaauuuugaaaauuauuagguuacuaugauaagguaguacaccgaaaagcucuaacgcucugucuuggacagagcguuaucuuuuuu

SEQ ID NO: 1573 - DNA, 6373 bp, Artificial Sequence
  pET9a-OMNI-103
taatacgactcactatagggagaccacaacggtttccctctagagagacaataaccctgataatgcttcaataatattgaaaaaggaagagtatgcctaagaagaagagaaaggtgggtacctctatcaagagcgactacttcctgggcctcgacatcggcacagattctatcggatgggccgtgacagaccccgagtaccacatcctgagaagaaagggcaaagccctgtggggcatcagactgttcgatgccgccaatactgccgccgagagaagaaccttccggaccagcagaagaagaatccagcggcggagacagcggatcagactgctgcaagagctgttcgccgaggaaatggttaagctggaccccggcttcttccagagactgagcgatagcgccttctggcaagaggacaagcaagagcagcagatctacagcctgtttacctgcgagaactacaccgacgtggactactacagagagtaccccaccatctaccacctgagaagcgccctgatccaagagaaaaaagagttcgacctgcggctgctgtatctggccctgcaccatctgatgaagcaccggggccactttctgttcaacggcagcatcaacaacgtgaccagcttccacaccaccttccagaccttcgccgactgcctgtacgacgagttcgacatcgagctggaatgcgacagcgaggacagattcgccgagatcctgaaggataagcacgccagaaagaccggcaagtgctctgagctggaaatcatctgccacatcgagaagtccaacaagcagctgaaagaactgttcaagctgatcaccggcatgaaggccagcctgagcgtggtgtttggagatgatgagctggccgagatcgagcacaacaagatcagcttcagcgagagcagctacgacgaagtgcggctggccctggaagatgagattcaagagagaaccggcatcctggacatcttccacgccgtgtattcttgggccatcctggccgatattctggaaggcggcgagtacgagggcaacagctatctgtctgtggccaaggtgtccacctacaagaagcacggcgacgacctgagactgctgagaacactcgtgcgcgagtactgccccgaccactacaagagctttttcagcgtgtccggcaaagagaactactgcgcctacgccggcacactgaagaagaacggcaagaagcagcccatcaagcggtgcagccaagaggacttctacaaggccctgaagaaactgctgaaccagatgcctaccgagcagcccgaagtgaaggatatcttcatcgagattgagaacggcaccttcctgcctctgcaagtgtccaaggacaacggcgtgatcccctaccaagtgaacaagatggaactcgagaagatcctgcagaacgccgaagagtacctgcctttcctgaagaacatcgacgaggaatgcggcaagaccgtgtccaagaagatcatcgacctgttcgagttcagaatcccctactacgtgggccctctgaataccgccaagggcgagaattgctggatggttcgaaaagaggccggcagaatctacccctggaacttcgatgagaaggtggacagagatcagagcgccgagaagttcatcagacggatgaccaaccagtgcacctacctgatccacgaggacgtggtgcctaagaacagcctgctgtactccgagttcatggtgctgaacgagctgaacaatgtgaagattcggagcgagaagctgcccgtggaactgaagcaggccatcgtgctggacctgtttaagaagcagaagcagatcacagggaagaagctgctcaactacctgaacgccaacggctacgacgtgaagaaagaggacctgagcggcttcgacggcaacttcaagtccagcctgtccagctacctgactctgaagaaggtgttcggagaggaactggacaagtacagcgtgcagcagatggccgaggacatcatcctgtggatcaccctgtatggcgacgatcagaaaatgctgcggagagtgatccggaagcactacgagcagcagctgtctgaggaacagatcctgagcctgagcaagctgaagttccaaggctggggcagactgtctagacggctgctctctgaaatggaaggcgtggactgtgaaaccggcgagtgcatgacagtgatgcagggcctgagaaacacccagaacaacctgatgcagctgctgagccagcagttcagcttcatggaactgatcgaggaagagaacgggaactactacgtcgacgagatcacctacgacaacctggtcaaggacatggtcatcagccctagcgtgaaaagggccgtgtggcagacagtgcagatcgtggaagaaatcaagggcgtgatgggatgccagcctaagaaaatcttcgtggaaatggcccgcagcgacgaagagaagaaacggaccgtgtctcggaaggatcggctgctggaagcctacgacgccatcaaggatgaggcccggcaatggcaagaagaactgcagaaatactccgacggcgatttcaaggccatcaagctgtacctgtactacacccagatgggccagtgcatgtacaccggcagaaaaatcgatctgtcccagctgaacgacgccaccgtgtgggatagagatcacatctaccctcagagcaagaccaaggacgacagcctggacaatctggtgctggtggatagatccgtgaatgccaagaaaagcgacggcatgctgagccccgagatccagcagagaatgagagccacctggaagtacctgaaagaaaagaagctcatcagcgagaagaagtacgagcggctgaccagagtgtcccctctgacagatgaagaactggccggcttcatcaaccggcagctggtggaaacaagacagagcagcaaagccgtggccacactgctgaagagggtgtacgatgaggccgagattgtgtatgtgaaggccgaggccgtgtctaacttccggcgggataacctggactacatcaaagtgcgggacctgaacgactaccaccacgccaaggacgcctaccagaacatcgtcgtgggcaacgtgttccacgagaagtttaccagcaatcccctgcggtggctgaaaaacaaccccaacaccaagtactccctcaaccagatgttcaacttcgacctggaaaagaacggcgtggtcatctggaagagaggcaaggccggctccattaagtgtgtggaagagacactgaagcggaacgacatcctgttcaccagatacgctttctgcaacaaaggcggcttctttaatcagatgctgaccgccgctccagaggataagacaaaggccaaaggcctggtgcctatcaagaaaggcatggaaacctggaaatacggcggctacaccagcgtgacccctagccactttatgctggtggccagcaaggacaagaagggaaaagagatccggaccatcgagacagtgcccctgtaccggtggaaagagttcaaagagaatcccgacgctctgctccagtactgcagagagttctacggcctgaaagagcccaaggttctgatcccttgcatcaagaagaatgcccggctggtcgtgaacggcttccctatgcacctgaagggcagcaccggaaaacagctgattctgcagggtgccgtgcagctgtgcctgaacaacgagaacatcaagtacctcaagaaagtgacgaagtacctcgagtacaacgcccagcggagagacaagagaaccctgctcgaagttcgggaagtgaccggaatcaacaaagaggaaaacatccagctgtacgatgtgttcgtggacaagctgagcaacacaatctaccagtacagacccgccaatcctaaggacaacctcatcaagggccgcgagaaattcatcgagcttggcctggctgagcagtgcgtggtgctgggagaagtgctgcatctgttccagtgcaagcccctgaccagcgatctgacactgatcggcggaagccctaacaccggcaccatcaagatcaccaagaccatcagcaactgcaacgtggtcaagctgctgtcccagtctatcgccggcgtgaaagtccgcgagatcaacctgctgatcatcggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 1574 - DNA, 2783 bp, Artificial Sequence
  pShuttle Guide-T2-OMNI-103 V2
tacacggtgcctgactgcgttagcaatttaactgtgataaactaccgcattaaagcttatcgatgataagctgtcaacacatttccccgaaaagtgccacctgacgtcctcgagtcccgcataatcgaaatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgctaatacgactcactataggaagagcagagccttggtctcgtttgagagtagtgtaagaaattacactacaagttcaaataaaaatttattcaaatccatttgctacattgtgtagaatttaaagatctggcaacagatctttttttgaattctctagcataaccccttggggcctctaaacgggtcttgaggggttttttgacctaggctaggggatatattccgggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttca

SEQ ID NO: 1575 - DNA, 5009 bp, Artificial Sequence
  pbPOS T2 library
  misc_feature (3040)..(3047): n is a, c, g, or t
tcgagtctttacactttatgcttccggctcgtatgttgtgtggaattgtgagcggataacaatttcacacatgattacggattcaacgtcgtgactggtaaaacccgggcgttacccaacttaatcgccttgcagcacatccccctttcgccagcaggcgtaataaggaaaggattcatgtactatttgaaaaacacaaacttttggatgttcggtttattctttttcttttacttttttatcatgggagcctacttcccgtttttcccgatttggctacatgatatcaaccatatcagcaaaagtgatacgggtattatttttgccgctatttctctgttctcgctattattccaaccgctgtttggtctgctttctgacaaactcggtctacgcaaatacctgctgtggattattaccggcatgttagtgatgtttgcgccgttctttatttttatcttcgggccactgctgcagtacaacattttagtagggtcgattgttggtggtatttatctaggctttagttttaacgccggtgcgccagcagtagaggcatttattgagaaagtcagccggcgcagtaatttcgaatttggtcgcgcgcggatgtttggcagtgttggctgggcgctggttgcctcgattgtcgggatcatgttcaccattaataatcagtttgttttctggctgggctctggcagttgtctcatcctcgccgttttactctttttcgccaaaacggacgcgccctcaagtgccacggttgccaatgcggtaggtgccaaccattcggcatttagccttaagctggcactggaactgttcagacagccaaaactgtggtttttgtcactgtatgttattggcgtttcctccacctacgatgtttttgaccaacagtttgctaatttctttacttcgttctttgctaccggtgaacagggtacccgcgtatttggctacgtaacgacaatgggcgaattacttaacgcctcgattatgttctttgcgccactgatcattaatcgcatcggtgggaagaatgccctgctgctggctggcactattatgtctgtacgtattattggctcatcgttcgccacctcagcgctggaagtggttattctgaaaacgctgcatatgtttgaagtaccgttcctgctggtgggctcctttaaatatattactagtcagtttgaagtgcgtttttcagcgacgatttatctggtcagtttcagcttctttaagcaactggcgatgatttttatgtctgtactggcgggcaatatgtatgaaagcataggtttccaaggcgcttatctggtgctgggtctggtggcgctgggcttcaccttaatttccgtgttcacgcttagcggcccgggcccgctttccctgctgcgtcgtcaggtgaatgaagtcgcttaaaggcctcgatgcagctagcatgctaatctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggggtagcgattgaaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataatcttctagacatacaatggaagagcagagccttggtctcnnnnnnnnaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacc

SEQ ID NO: 1576 - protein, 9 aa, Artificial Sequence
  HA-tag
YPYDVPDYA

SEQ ID NO: 1577 - protein, 7 aa, Artificial Sequence
  NLS
PKKKRKV

SEQ ID NO: 1578 - protein, 19 aa, Artificial Sequence
  P2A
ATNFSLLKQAGDVEENPGP

SEQ ID NO: 1579 - protein, 236 aa, Artificial Sequence
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 1580 - DNA, 27 bp, Artificial Sequence
  HA-tag
tacccatacgatgttccagattacgct

SEQ ID NO: 1581 - DNA, 21 bp, Artificial Sequence
  NLS
ccaaaaaagaaaagaaaagtt

SEQ ID NO: 1582 - DNA, 57 bp, Artificial Sequence
  P2A
gccaccaacttcagcctgctgaagcaggccggcgacgtggaggagaaccccggcccc

SEQ ID NO: 1583 - DNA, 711 bp, Artificial Sequence
  mCherry
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag